(19) United States
(12) Patent Application Publication
(10) Pub. No.: US 2024/0188569 A1
(43) Pub. Date: Jun. 13, 2024

(54) NEW YEAST STRAIN AND ITS USES FOR THE CONTROL OF PHYTOPATHOGENS

(71) Applicant: AGROVENTURES LLC, Southbury, CT (US)

(72) Inventors: RAFFAELLO CASTORIA, Roma (IT); VINCENZO DE CICCO, Bari (BA) (IT); FILIPPO DE CURTIS, Larino (CB) (IT); BRUNO FOLCHI, Southbury, CT (US); GIUSEPPE IANIRI, Campobasso (CB) (IT); GIUSEPPE LIMA, Ferrazzano (CB) (IT); DAVIDE PALMIERI, Riccia (CB) (IT)

(73) Assignee: AGROVENTURES LLC, Southbury, CT (US)

(21) Appl. No.: 18/285,480

(22) PCT Filed: Apr. 11, 2022
(86) PCT No.: PCT/IB2022/053387

§ 371 Date: Oct. 3, 2023

(30) Foreign Application Priority Data
Apr. 12, 2021 (IT) 102021000009128

(51) Int. Cl. A01N 63/32; A01P 1/00; A01P 3/00; A01P 5/00; C12N 1/16; C12R 1/645

(52) U.S. Cl. CPC A01N 63/32; A01P 1/00; A01P 3/00; A01P 5/00; C12N 1/165; C12R 2001/645

(57) ABSTRACT

The present invention relates to a new yeast strain for the control of the main phytopathogens, which is effective in the prevention, suppression, treatment or control of a wide range of phytopathogens and related diseases affecting the whole plant (both the aerial part and the radical part).

The present invention regards a new yeast strain and its uses for the control of phytopathogens. Importantly, the present invention concerns a new yeast strain for the control of the main phytopathogens, which is effective in the prevention, treatment and control of a wide range of phytopathogens and the related diseases affecting the whole plant (both the aerial and root parts).

It is well known that crop plants can be affected by various fungal, bacterial and viral diseases, as well as by numerous harmful insects that can also be vectors of phytopathogens.

Currently, plant protection is mainly based on the use of chemical pesticides. However, concerns about the emergence of resistance to chemical active ingredients in pathogen populations and the impact of these chemicals on human and animal health and on the environment, require the development of alternative plant protection approaches, such as biological and integrated control of plant diseases. Integrated pest management (IPM) consists of the combined use of both chemical and biological plant protection products along with agronomic measures useful in controlling plant pathogens, i.e. that are not conducive to pathogens attack. Biological management (i.e. biological control or biocontrol), on the other hand, mainly relies on the use of products formulated with microbial antagonists, plant-derived compounds or semiochemicals, which generally are naturally occurring and pose low risks to human health and the environment. In the last decades, the use of microorganisms for plant protection has resulted in a number of well-established commercial products, mainly based on soil-borne bacteria and fungi, which mainly act via mycoparasitism and the production of natural compounds, such as antibiotics or secondary metabolites with antimicrobial activity. The use of microbial antagonistic agents aims to increase the range of biocontrol formulates with low or no environmental impact, which can replace or integrate chemical active ingredients for which risk mitigation measures are being enforced for reducing their use or even withdrawing these chemicals from the market.

Some ascomycetous and basidiomycetous yeast isolates are of particular interest for various agricultural applications, including as biocontrol agents (BCAs) to counteract plant pathogens. Epiphytic fungal microflora abundantly populates the aerial parts of plant organs, being adapted to a wide range of environmental stresses. These natural features make yeasts and yeast-like fungi excellent candidates for the development of plant protection products for the control of plant pathogens, both in the field and during storage. Several yeast species with antagonistic properties against different pathogens have been selected and characterized for commercial use as BCAs e.g. Candida oleophila (Aspire®, Ecogen, Langhorne, PA, USA; Nexy®, Lesaffre-Bionext, France), Cryptococcus albidus (YieldPlus®, Lallemand, Montreal, Canada), Candida sake (Candifruit®, IRTA, Lleida, Spain), Metschnikowia fructicola (Shemer®, Bayer, Leverkusen, Germany) and Aureobasidium pullulans (Boniprotect®, Biofa, Musingen, Germany), the latter an yeast-like fungus.

For the commercial registration of BCA-based products, biological, ecological and toxicological studies are required to demonstrate that the selected microorganisms are effective against plant pathogens and harmless to humans, animals and the environment.

However, the range of efficacy for many biocontrol yeasts is too narrow both in terms of target pathogens and in terms of cultures. In fact, most of microbial-based formulates have extremely reduced scope of application, thus leading to the necessity of an integrated use with other active ingredients. Furthermore, the use of combination of biocontrol microorganisms with synthetic chemical active ingredients has a major constraint in the limited ability of BCAs to tolerate chemical active ingredients. Therefore, there are numerous technical restrictions for the combinations of most microbial species used as BCAs with synthetic chemicals, which further limits the use of these products.

In the light of this situation, it appears evident the need for using new BCAs that can solve the above-mentioned issues and represent alternatives to the ones presently marketed.

In this context, the present invention represents a possible solution, since it consists of the formulate of a new microorganism to be used as a biocontrol agent of the main plant pathogens.

According to the present invention, it has now been demonstrated that the yeast species Papiliotrema terrestris and its extracellularly secreted substances can be advantageously used against plant pathogens.

In particular, the Applicant has now isolated a new yeast strain (PT22AV), belonging to the basidiomycete yeast species Papiliotrema terrestris. The aforementioned yeast strain was filed for patent purposes in according to the procedures laid down in the Budapest Treaty, on 26 Oct. 2020, at the Westerdijk Fungal Biodiversity Institute (CBS), depositor AgroVentures srl, with access number provided by the International patent filing Authority CBS147138 and with identification reference provided by the depositor of PT22AV.

In line with the invention, Table 1 shows the taxonomic classification of the PT22AV strain of Papiliotrema terrestris.

TABLE 1
Taxonomical classification of PT22AV
Scientific classification

| | |
|---|---|
| Kingdom | Fungi |
| Phylum | Basidiomycota |
| Subphylum | Agaricomycotina |
| Class | Tremellomycetes |
| Subclass | Tremellomycetidae |
| Order | Tremellales |
| Family | Rhynchogastremataceae |
| Genus | Papiliotrema |
| Species | Papiliotrema terrestris |

Synonyms
Cryptococcus terrestris
Strain
Papiliotrema terrestris PT22AV

The genus Papiliotrema (Class Tremellomycetes, Order Tremellales, Family Rhynchogastremataceae) was first described in 2002 with the aim of classifying a new microbial species, Papiliotrema bandonii.

Recently, the taxonomic reclassification, involving many species of basidiomycetous yeasts, has transferred numerous species from genus Cryptococcus to genus Papiliotrema. At present, this new fungal genus includes about 24 species and among these many are described as biological control agents, such as P. laurentii (formerly C. laurentii). Papiliotrema terrestris is a relatively new species, in fact it was first described in 2015.

In particular, as shown in the examples reported below, the PT22AV strain of Papiliotrema terrestris, isolated according to the present invention, has shown better or comparable performances as a biocontrol agent as compared to already known chemical and biological products. In particular, fresh, dried or dehydrated PT22AV cells, applied in biological or integrated protocols for seed coating, in seedbed, in the field or in the post-harvest stage, showed a reduction in the disease incidence that was comparable to that obtained with synthetic chemical products.

As shown below, the PT22AV strain of Papiliotrema terrestris according to the present invention was tested in efficacy and selectivity assays conducted for a period of 3 years (2018-2019-2020) in 4 EU countries on 6 crops against a wide range of biotrophic and necrotrophic fungal pathogens, in the field and in the post-harvest. Different formulates based on strain PT22AV were tested, which were different from each other for composition and concentration of yeast cells, and were obtained through different approaches and technologies.

On the basis of the obtained results, strain PT22AV of Papiliotrema terrestris according to the present invention has proved to be a better biocontrol agent against plant pathogens as compared to:
a) commercial microbial formulates based on bacteria (e.g. Bacillus subtilis and B. amyloliquefaciens), yeasts (e.g. Aureobasidium pullulans) and fungi (e.g. Trichoderma spp.);
b) numerous commercially established chemical plant protection products.

As regards the comparison with formulates based on bacteria and yeasts (point a), Papiliotrema terrestris PT22AV, according to the present invention, has proved to yield better performances since it can be used at much lower cell concentrations with the same efficacy. Overall, strain PT22AV according to the present invention, used alone or in combination with other agrochemicals (fungicides, adjuvants, fertilizers, biostimulants, etc.) was demonstrated to be much more effective than other commonly used microbe-based formulates (e.g. Bacillus spp.) in the control of phytopathogenic fungi (including mycotoxigenic ones) and other plant pathogens, when applied both in the field and post-harvest, both in the aerial and in the root part. Furthermore, the strain according to the present invention, unlike chemical formulates indicated above (b), does not pose any risk of selecting populations of resistant pathogens, is safer for humans and the environment, acts on a wider range of crops, diseases, phenological stages of plants, has a better adaptability to environmental conditions, can be better used in synergistic integration with various chemical or biological fungicides, adjuvants and other agrochemicals.

On the other hand, as regards the comparison with chemical products (point b), the strain according to the present invention has shown comparable or even higher performances as compared to a wide range of chemical fungicides. Furthermore, according to the present invention, P. terrestris PT22AV does not produce any toxic chemical residues unlike chemical products. In addition, the strain can be applied both before and after harvest, does not allow the onset of resistance in pathogens' populations, is compatible with many agrochemicals when used in combination when used in combination with these products, and can be used both in organic farming and in integrated pest management.

According to the present invention, the efficacy of the treatment based on strain PT22AV has been clearly demonstrated for both fresh and dehydrated cells.

According to the present invention, formulates including the strain PT22AV are particularly advantageous since, if applied to crops, they actively protect the plant, promote its growth, stimulate systemic resistance against pathogens, reduce the production and accumulation of mycotoxins, mitigate the harmful effects of nematodes and other biotic and abiotic stressors. Furthermore, according to the present invention, strain PT22AV of P. terrestris is highly active in preventing, suppressing and controlling a wide range of diseases/pathogens affecting the whole plant (aerial parts and roots), with the possibility of application in the field, in post-harvest, in the soil and for seed coating. In particular, strain PT22AV according to the present invention can be advantageously used for root and seed treatment and for the entire aerial parts (including flowers and fruits) of plants, and can be applied alone or in synergistic combinations with other products for agriculture, in particular with pesticides (acaricides, bactericides, fungicides, insecticides, herbicides, algaecides, molluscicides, nematicides, rodenticides, plant growth regulators, repellents, semiochemicals, virucides), bio-pesticides, adjuvants and other agrochemicals (additives, fertilizers, biostimulants).

Therefore, according to the present invention, P. terrestris PT22AV, compared to other known biocontrol yeasts, can be advantageously used against a wide range of pathogens in different types of crops and is tolerant towards synthetic chemical active principles, which makes it possible its effective use in combinations with these chemicals, as shown in the examples below.

In particular, the PT22AV strain of P. terrestris, according to the present invention, can be advantageously used as a biocontrol agent (BCA) for the management of plant pathogens both in organic agriculture and in integrated disease management (IDM).

Furthermore, according to the present invention, it has been found that strain PT22AV has a very active secondary metabolism, and secretes a series of secondary metabolites in the extracellular environment. Among these, extracellular polymers are of particular importance, a complex group of substances largely made up of polysaccharides. The composition and production of these polymers are very complex and it has been observed that it is conditioned by several factors, including the composition of the growth medium, pH, temperature and oxygenation during biomass production. According to the present invention, it has been found that the extracellular polymers produced by P. terrestris strain PT22AV are mainly composed of carbohydrates (50-90%); the most represented monomers are: mannose, xylose, glucose, galactose, arabinose. The structure of these polymers is very similar to starch and glycogen, it is indeed organized in a main chain consisting of monomeric units linked through α-(1→3) bonds and branches anchored through β-(1→2) bonds. These compounds are mainly produced under conditions of trophic stress and in all conditions limiting cell growth. These polymers are active in the stimulation of plant metabolism, in inducing resistance against pathogens, to improve the chemical, physical and biological characteristics of the soil, to promote the healing of wounds in plants, and if they are distributed on the aerial parts they constitute a physical obstacle to penetration of pathogens.

Therefore, according to the present invention, the aforementioned extracellular substances produced by the PT22AV yeast strain can be advantageously used against plant pathogens.

Scientific papers on molecular taxonomy have highlighted a high genetic homogeneity within the species P. terrestris; this indicates that in this species, the rate of genetic recombination linked to sexual reproduction is low, and that asexual reproduction is prevalent, which is a guarantee of genetic and phenotypic stability, and therefore of constant high performance in the field, in other application and also over time.

No technical and/or scientific publications have so far documented evidence of both biocontrol and biostimulant activities of P. terrestris species in the pre-harvest stage—in the ground or in the open field, in soil applications, on seeds and on aerial plants parts. Therefore, according to the present invention, the biostimulant activity of the yeast species P. terrestris, in particular of the strain PT22AV, in pre-harvest stage, is shown for the first time (see Example 5).

Moreover, according to the present invention, the biocontrol activity by the yeast species P. terrestris, in particular of strain PT22AV, is shown for the first time in pre-harvest stage. According to the international scientific literature, the only reported biocontrol activity by the species P. terrestris regards strain LS28 (previously classified as Cryptococcus laurentii), which was not applied in the open field but it was only applied on the harvested products (post-harvest) against post-harvest fungal pathogens. Nevertheless, biocontrol activity by PT22AV strain is significantly higher than the biocontrol activity by LS28, as compared by the Applicant. As shown in the comparative experimental data reported below (see Example 6), the efficacy of PT22AV is significantly higher than the efficacy of LS28. In addition, PT22AV showed a significantly greater ability to resist a wide range of abiotic stresses by PT22AV as compared to LS28.

The PT22AV strain, according to the present invention, is safe for humans and the environment, its manufacturing process has no environmental impact and satisfies green and circular economy principles, with manufacturing by-products to be used as soil organic conditioners and with possibility to use food industry by-products such as molasse for its fermentation process. The efficacy, selectivity, toxicological and eco-toxicological properties of the formulations based on the PT22AV strain have been extensively tested under GAP-GLP conditions (as required by current legislation regarding the European registration of microbial-based pesticides).

As far as human toxicology is concerned, Papiliotrema terrestris is a naturally occurring and non-pathogenic yeast species. In this regard, Ke et al. (2018) report that Papiliotrema terrestris was no-pathogenic in Sprague-Dawley rats after intravenous (IV) administration of 2.4×10⁸ CFU and oral administration of 1.3×10⁹ CFU. The genotoxic results for the galactosidase enzyme concentrate are negative, both in a bacterial reverse mutation test (Ames's test) and in a chromosome aberration test in cultured Chinese hamster lung fibroblast (CHL/IU) cells. In addition, in the 13-week gavage study in Sprague-Dawley rats, no adverse effects were observed in any of the tested groups and a No Observed Adverse Effects Level (NOAEL) of 2000 mg/kg bw/day [total organic solids (TOS) 1800 mg/kg bw/day)] was established, which was the highest dose tested. Allergenicity sequence analysis revealed no evidence that galactosidase enzyme is an allergen. The data presented in this study support the conclusion that galactosidase produced by P. terrestris is safe for use in food production. The Applicant has carried out additional toxicological studies (data not shown), which confirm that strain PT22AV according to the present invention has a favorable toxicological profile based on values obtained from oral, pulmonary, intraperitoneal, dermal, eye irritation and other relevant toxicological tests.

As regards environmental toxicology, the Applicant has carried out various studies (data not shown), necessary for the commercial registration process in Europe and in the United States, which confirm that the formulations based on the PT22AV strain are safe for the environment.

Studies include evaluations of birds, mammals, non-target arthropods such as bees, predatory mites, soil organisms such as earthworms and others, aquatic organisms such as fish, daphnia and algae, as well as other non-target organisms, for which selectivity studies are required by regulatory bodies in Europe and USA.

According to the present invention, the P. terrestris strain PT22AV can be effectively used as a broad-spectrum fungicide (in terms both of crop plants and of plant pathogens) in organic farming, as well as for integrated pest management programs.

Furthermore, the ability to quickly colonize the plant surfaces, natural openings and wounds advantageously enables the PT22AV strain to effectively counteract even the phytopathogenic bacteria that penetrate their hosts through these openings (e.g. Pseudomonas spp., Xanthomonas spp., Clavibacter spp., Ralstonia spp., Erwinia spp). In particular, the strain PT22AV produces a large quantity of polysaccharides in extracellular environments during growth both in vitro and in vivo. This very complex matrix protects the strain from abiotic stressors, improves adhesion to surfaces and triggers plant systemic resistance, improving tolerance to a wide range of pathogens, including viruses.

In addition, the P. terrestris species, in particular the PT22AV strain, according to the present invention, can be advantageously applied also to the soil, in particular for the improvement of the soil structure itself and for nematodes control. In fact, the application of the PT22AV strain to the soil produces a series of beneficial effects, including a positive action on the soil and rhizosphere microbiome. The polysaccharide matrix, which facilitates the formation of aggregates and therefore improves the soil structure, is a source of nutrients for useful bacteria, microfauna and predatory protists, and contributes to the ecological biogeochemical processes that are essential for the preservation of the soil ecosystem. Furthermore, formulations including the PT22AV strain according to the present invention are particularly advantageous since, if applied to crops, they actively protect the plant and promote its growth and resistance to biotic stressors, such as damage from nematodes. In particular, when applied as a root treatment, the PT22AV strain while partially reducing the proliferation of nematodes it is also especially effective in limiting their harmful effects, as shown by measuring plant height (see Example 7). Possible nematodes species are Meloidogyne spp., Heterodera spp., Globodera spp., Belonolaimus spp., Pratylenchus spp., Rotylenchulus spp., Trichodorus spp., Paratylenchus spp. etc.

The PT22AV strain is effective at a broad spectrum of concentrations by improving the overall health of plants.

The mechanisms underlying the antagonistic action of the PT22AV strain according to the present invention advantageously allow its application on a broad spectrum of host plants and pathogens (fungi, bacteria and viruses), and a high flexibility in its application both in terms of time (all the phenological phases) and in terms of the treated organs (seed, root, epigeous plant parts, flower, fruit). Furthermore, the ability to adapt to very heterogeneous environmental conditions (humidity, temperature, UV irradiation etc.) allows its application in different climatic areas.

Underlying the efficacy of the PT22AV strain in controlling plant diseases is the ability to survive and persist on plant surface or on wounded tissues for long periods. In particular, the tolerance to environmental stresses such as dehydration, oxidative stress, UV radiation, osmotic stress and tolerance to fungicides allow the adaptation of the PT22AV strain to the application environment.

According to the present invention, the plant protection against many fungal pathogens exerted by P. terrestris PT22AV is mainly preventive. In particular, the application of the live cells of the strain prevents the establishment of the infection process in the early stages of plant-pathogen interaction (e.g. conidia germination, germ tube elongation, infection site colonization). Furthermore, bio-protection is the result of several mechanisms with different relevance, which regulate the interactions both with pathogens and with other microorganisms that populate the same ecological niche.

The modes of action that characterize the biocontrol activity of the PT22AV yeast strain according to the present invention are listed below.
Wound competence: for most necrotrophic fungi, wounded plant tissues represent the main penetration site to establish the infectious process. As already widely described in the literature, plant tissues respond to a wide range of stresses by triggering oxidative nature processes. Among these, a robust oxidative response is triggered by wounded plant tissues, which brings along the accumulation of reactive oxygen species (mainly superoxide anion and hydrogen peroxide) and other oxidizing substances. Studies carried out by the Applicant demonstrate how the biological control agent PT22AV effectively colonizes injured tissues by resisting the oxidative stress exerted by reactive oxygen species (ROS: superoxide anion O₂—, and hydrogen peroxide, H₂O₂) generated by plant tissues as a consequence of wounding. The selected yeast strain, according to the invention, tolerates oxidative stressors through two enzymes (superoxide dismutase and catalase). Resistance to oxidative stress can be considered the first mechanism implemented by PT22AV against post-harvest wound pathogens. In particular, the Applicant has demonstrated the PT22AV strain strong ability to resist oxidative phenomena by the assessment of the population dynamics in artificial wounds on apple, which showed how this microorganism is able to effectively colonize this environment. The strain population was assessed by plating on nutrient agar medium yeast samples withdrawn from the wounded tissue inoculated with the PT22AV strain. From this analysis it emerged that the PT22AV strain colonizes the wound very quickly, doubling in concentration within 6 hours from inoculation. Furthermore, strain resistance to oxidative stress was further investigated by the Applicant by evaluating the growth of the microorganism in the presence of hydrogen peroxide. For this purpose, increasing concentrations of hydrogen peroxide were added to the NYDA agar plates, subsequently inoculated with a suspension of PT22AV strain cells. From this analysis it emerged that this microorganism effectively withstands hydrogen peroxide concentrations up to 30 mM, therefore to a level of oxidative stress much higher than the one that is produced by wounding of a plant tissue.
Competition for space and nutrients: PT22AV acts by efficiently competing for space and nutrients with fungal pathogens. Papiliotrema terrestris isolate effectively inhibit the development of the pathogen in its early growth stages by preventing the infection process. In vivo experiments, carried out by the Applicant, have highlighted how the addition of exogenous nutrients in artificial wounds drastically reduces the biocontrol efficacy of the yeast. Furthermore, in space competition, the PT22AV yeast strain is aided by the formation of an extracellular polysaccharide capsule which can promote adhesion to the fruit surface, thus preventing contact between host fruit and pathogen (FIG. 2). Therefore, competition for space and nutrients is a key mechanism of P. terrestris PT22AV in its antagonism against necrotrophic fungi and is closely related to oxidative stress resistance, hence to the ability to colonize plant tissues. Effective and timely space competition is a major trait of PT22AV making it effective against any disease and any plant culture (i.e. it confers this yeast a wide spectrum biocontrol activity). More specifically, efficacious competition of PT22AV for space (and consequently for nutrients) is a highly complex phenotypic trait relying on many genes, which enables this BCA to exert an action that is analogous to multi-site fungicides, but with a much wider range of mechanisms of action and number of involved genes; in the analogy with these fungicides, in fact, competition makes it very unlikely, if not impossible in the case of PT22AV, the selection of pathogen populations that are resistant to this BCA.
β-1,3-glucanase activity: P. terrestris strain PT22AV, in addition to competition for space and nutrients, can directly interact with fungal pathogens through the degradation of their cell wall mediated by the production of the extracellular β-1,3-glucanase enzyme. In vitro studies carried out by the Applicant have shown that P. terrestris PT22AV is able to produce significantly higher levels of extracellular β-1,3-glucanase when cultured in the presence of hyphal cell walls of the pathogens P. expansum and B. cinerea as sole sources of carbon. This mechanism is to be considered second level in terms of relevance compared to the aforementioned mechanisms.
No antibiosis: metabolomic studies carried out by the Applicant have shown that, unlike other microorganisms active against plant pathogens and registered as the active ingredient in microbial-based formulates (in particular bacteria) P. terrestris PT22AV does not produce molecules with antifungal activity. Under experimental conditions, the antibiosis appears not to be involved in the antagonist activity exerted by strain PT22AV, thus ruling out the possibility that fungal pathogens develop resistance. This makes PT22AV a much safer and more sustainable BCA than bacteria-based formulates, the number of which is currently higher than other microbial species (fungi and yeasts).
Plant resistance induction: plants have an innate “immune system” to recognize and respond to pathogens (fungi, bacteria and viruses); this plant “immune response” can be triggered by various beneficial microorganisms inducing resistance both locally and systemically. Biocontrol yeasts can elicit plant systemic resistance against a broad range of pathogens and this action is implicit in their biocontrol activity. The induction of resistance can be triggered by microbial cells, parts of them or even by the molecules secreted in the extracellular environment once recognized by plants. Papiliotrema terrestris PT22AV produces many extracellular substances that can trigger a systemic defense response in the host plant thus improving tolerance to pathogens. This extracellular substance production activity is unmatched by any other commercially available biocontrol microorganism.
Adhesion to the plant surface: during biomass production and after application in the field, the PT22AV strain produces an abundant matrix of polysaccharides that allows yeast cells to adhere to solid surfaces. This extracellular matrix promotes the PT22AV adhesion to plants and wounded tissues, improves resistance to washout caused by rain and minimizes the negative effects of biotic and abiotic stressors. This activity is much higher than that of any other BCA commercially available or described in the literature.

It is therefore a specific object of the present invention a yeast strain belonging to Papiliotrema terrestris species, said strain being deposited at the Westerdijk Fungal Biodiversity Institute with deposit number CBS147138, hereinafter also referred to as PT22AV strain. According to the present invention, said yeast strain can be in a form selected from fresh cells, dried cells, dehydrated cells, devitalized cells, inactivated cells, frozen cells or cells in aqueous suspension, including derivatives (mainly extracellular polysaccharides) of the cell biomass production process.

The present invention also relates to a phytosanitary composition for agricultural use, in particular for controlling phytopathogens in plants or crops, said composition comprising the PT22av yeast strain of the Papiliotrema terrestris species defined above, as the active principle, together with one or more excipients or phytopharmacologically acceptable adjuvants.

According to the present invention, said composition can be in liquid or solid form.

Furthermore, according to the present invention, the concentration of said yeast strain within the composition can range from 10³ CFU to 10¹³ CFU per gram of solid composition or from 10² CFU to 10¹² CFU per mL of liquid composition. Solid and liquid formulates at a concentration higher than 10⁷ CFU per gram or mL are mainly suitable for foliar and post-harvest applications, while for formulates with a concentration lower than 10⁷ CFU per gram or mL they can be used for soil treatments, enrichment of compost or other substrates for sowing, transplanting or growing plants and seed coating.

The concentration of the active ingredient in the formulates according to the invention is expressed as colony forming units (CFU) per gram or mL. The quantification can also be performed by other techniques such as real-time PCR, flow-cytometry, etc.

The PT22AV strain concentration in solutions for fertigation or in hydroponic cultivation etc can range between 10² and 10¹² CFU per mL of solution. In distribution systems that involve the use of pollinating insects, the concentration of yeast in the trays dispenser can range between 10² and 10¹².

The composition according to the present invention can be in the form of tablets, capsules, granules, pellets, powder, such as dry powder or wettable powder, wettable granules, fluid, dry fluid, emulsion, suspension, solution, dispersion.

In particular, a composition according to the present invention can be in the form of: Aerosol (A); bait for pollinators dispersion (B); Dust (D); Dry flowable (DF); Emulsifiable (E); Emulsifiable concentrate (EC); Flowable (FL); Granule (G); Microencapsulated (M); Pellet (P); Soluble powder (SP); Wettable powder (WP); Water-dispersible granule (WG/WDG).

More specifically, composition can be in the form of: grain bait (AB); bait concentrate (CB); capsule suspension for seed treatment (CF); encapsulated granule (CG); capsule suspension (CS); dispersible concentrate (DC); dispersible powder (DP); powder for dry seed treatment (DS); emulsifiable concentrate (EC); emulsion, water-in-oil (EO); emulsion for seed treatment (ES); emulsion, oil in water (EW); fine granule (FG); flowable concentrate for seed treatment (FS); granular bait (GB); microgranule (GG); flo-dust (GP); Granule (GR); solution for seed treatment (LS); micro-emulsion (ME); microgranule (MG); oil dispersion (OD); oil miscible flowable concentrate (OF); oil miscible liquid (OL); oil dispersible powder (OP); paste (PA); seed coated with pesticide (PS); suspension concentrate (SC); suspo-emulsion (SE); water soluble granules (SG); soluble concentrate (SL); water soluble powder (SP); water soluble powder for seed treatment (SS); ultra-low volume suspension (SU); tablet (TB); technical material (TC); technical concentrate (TK); water-dispersible granular (WG/WDG); wettable powder (WP); wettable dispersible powder for slurry seed treatment (WS).

According to an embodiment of the present invention, said composition can further comprise one or more chemical compounds, for example synthetic chemical compounds, and/or one or more biological agents, for example microorganisms such as bacteria, yeasts, fungi, viruses, algae, said one or more chemical compounds and said one or more biological agents being selected from among fungicides, insecticides, fertilizers, biostimulants (e.g. humic and fulvic acids, protein hydrolysates, seaweed and plant extracts, chitosan and other biopolymers, inorganic compounds, beneficial microbes), macronutrients (e.g. nitrogen (N), phosphorous (P) and potassium (K)), micronutrients, elicitors, phyto-regulators, microbial biocontrol agents, plant growth regulators, foliar nutrients, antibiotics, herbicides, acaricides, food additives (e.g. all compounds included in following categories: antioxidants, colors, preservatives and flour treatment agents), adjuvants (e.g. emulsifiers, wetting agents, dispersants, defoarmers, chelating agents, neutralizers, solvents), microorganisms of agri-food interest.

According to the present invention, the excipients that can be used in the composition are for example co-formulants based on minerals (mineral powders) or organic (simple, complex or cyclic sugars), dispersants, emulsifiers, wetting agents, stabilizers, synergists, corroborants.

The aforementioned composition, further comprising one or more chemical compounds and/or one or more biological agents and/or one or more co-formulants, can be a tank-mix or a co-formulation.

In particular:
said foliar nutrients can be selected from nitrogen, phosphorus, potassium, calcium, sulfur, magnesium;
said micronutrients can be selected from iron, boron, chlorine, manganese, zinc, copper, molybdenum and nickel;
said insecticides can be selected from inorganic compounds, oils, botanical extracts, organochlorines, organophosphates, carbamates, neonicotinoids, avermectins, organosulfur, organotins, synthetic pyrethroids;
said elicitors can be chosen from chemical synthesis elicitors, such as salicylic acid, methyl salicylate, benzothiadiazole, benzoic acid and chitosan, or from plant extracts elicitors, such as tannins, citrus oils, bioflavonoids, such as dihydroquercetin, polysaccharics, arabino-galactans, polysaccharides and other plant extracts;
said plant growth regulators can be chosen from (international nomenclature) auxins, gibberellins, cytokinins, ethilene, abscissic acid, ancymidol; chlormequat; chloro IPC; daminozide; flurprimidol; hydrogen cyhanamide, cyanamide (H₂CN₂); mefluidide; mepiquat chlorure; paclobutrozol; proexadione calcium; succinic acid (SADH), forchlorfenuron.

According to an embodiment of the present invention, said composition is a fungicidal composition. For example, the fungicidal action is carried out by the specific strain and/or by further fungicides added to the composition.

According to the present invention, the fungicides can belong for example to strobirulins, benzimidazoles, sterol inhibitors, dicarboxamides, dithiocarbamates, inorganic fungicides, phenylpyrroles or their combinations, and to other chemical groups.

In particular, said fungicides (chemical and/or biological) can be selected from among (international nomenclature) 2-methoxyethylmercury chloride, 2-phenylphenol, 3-ethoxypropyl mercury bromide, 8-hydroxyquinoline sulfate, 8-phenylmercurioxyquinoline, acibenzolar, acibenzolar-S-methyl, acylamino acid fungicides, acypetacs, Adavelt, Agrobacterium radiobacter K84, aldimorph, allyl alcohol, ametoctradin, aminopyrifen, amisulbrom, ampropylfos, anilazine, aureofungin, azaconazole, azithiram, azoxystrobin, Bacillus amyloliquefaciens (formerly subtilis) strain QST713, Bacillus amyloliquefaciens strain AH2, strain IT-45, strain FZB24, strain MBI600, strain D747, Bacillus mycoides isolate J, Bacillus nakamurai strain F727, Bacillus pumilus strain QST 2808, Bacillus subtilis var. amyloliquefaciens strain FZB24, Bacillus subtilis strain AFS032321, strain GB03, strain IAB/BS03, barium polysulfide, benalaxyl-M (=kiralaxyl), benodanil, benomyl, benquinox, bentaluron, benthiavalicarb, Benthiavalicarb-isopropyl, benzalkonium chloride, benzamacril, benzamide fungicides, benzamorf, benzohydroxamic acid, benzovindiflupyr, bethoxazin, binapacryl, biphenyl, bitertanol, bithionol, bixafen, blasticidin-S, Bordeaux mixture, boric acid, boscalid, bromuconazole, bupirimate, Burgundy mixture, buthiobate, calcium polysulfide, captafol, captan, carbamorph, carbendazim, carboxin, carpropamid, carvone, cell walls of Saccharomyces cerevisiae strain LAS117, Cheshunt mixture, chinomethionat, chlobenthiazone, chloraniformethan, chloranil, chlorfenazole, chlorodinitronaphthalene, chloroneb, chloropicrin, Chlorothalonil, chlorquinox, chlozolinate, ciclopirox, climbazole, Clonostachys rosea strain CR-7, Coniothyrium minitans, copper hydroxide, copper naphthenate, copper oleate, copper oxychloride, copper soap, copper sulfate, basic, copper zinc chromate, copper(II) acetate, copper(II) carbonate, basic, copper(II) sulfate, coumoxystrobin, cufraneb, cuprobam, cuprous oxide, cyazofamid, cyclafuramid, cyclobutrifluram, cycloheximide, cyflufenamid, cymoxanil, cypendazole, cyproconazole, cyprodinil, Cyprofuram, dazomet, DBCP, debacarb, decafentin, dehydroacetic acid, dichlobentiazox, dichlofluanid, dichlone, dichlorophen, dichlorophenyl, dichlozoline, diclobutrazol, diclocymet, diclomezine, dicloran, diethofencarb, diethyl pyrocarbonate, difenoconazole, diflumetorim, dimethachlone, dimethirimol, dimethomorph, dimoxystrobin, diniconazole, diniconazole-M, dinobuton, dinocap, dinocap-4, dinocap-6, dinocton, dinopenton, dinosulfon, dinoterbon, diphenylamine, Dipymetitrone, dipyrithione, disulfiram, ditalimfos, dithianon, DNOC, dodemorph, dodicin, dodine, donatodine, drazoxolon, edifenphos, enestrobin, enestroburin, enoxastrobin, epoxiconazole, etaconazole, etem, ethaboxam, ethirimol, ethoxyquin, ethylene oxide, ethylmercury 2,3-dihydroxypropyl mercaptide, ethylmercury acetate, ethylmercury bromide, ethylmercury chloride, ethylmercury phosphate, etridiazole, extract from Melaleuca alternifolia (tea tree), extract from Reynoutria sachalinensis (giant knotweed), extract from the cotyledons of lupine plantlets (“BLAD”), extract of Swinglea glutinosa, F500, famoxadone, fenamidone, fenaminosulf, fenaminstrobin, fenamistrobin, fenapanil, fenarimol, fenazaquin, fenbuconazole, fenfuram, fenhexamid, fenitropan, fenoxanil, fenpiclonil, fenpicoxamid, fenpropidin, fenpropimorph, fenpyrazamine, fentin, fentin acetate, fentin chloride, fentin hydroxide, ferbam, ferimzone, Florylpicoxamid, fluazaindolizine, fluazinam, fludioxonil, flufenoxystrobin, fluindapyr, flumorph, fluopicolide, fluopimomide, fluopyram, fluoroimide, fluotrimazole, fluoxapiprolin, fluoxastrobin, fluquinconazole, flurozolamide, flusilazole, flusulfamide, Flutianil, flutolanil, flutriafol, fluxapyroxad, folpet, fosetyl, fosetyl-Al, fthalide (phthalide), fuberidazole, furalaxyl, furametpyr, furcarbanil, furfural, furmecyclox, furophanate, Gliocladium catenulatum J1446, Gliocladium virens GL-21, glyodin, griseofulvin, guazatine, halacrinate, hexachlorobenzene, hexachlorophene, hexaconazole, hexylthiofos, hymexazol, imazalil, imibenconazole, iminoctadine, Inatreq (fenpicoxamid), inorganic oils, inpyrfluxam, iodocarb, ipconazole, Ipflufenoquin, iprobenfos, iprodione, iprovalicarb, isofetamid, isoflucypram, isopropanol azole, isoprothiolane, isopyrazam, isotianil, isovaledione, Jun Si Qi, kasugamycin, kresoxim-methyl, laminarin, Lime sulfur (lime sulphur), mancopper, mancozeb, mandestrobin, mandipropamid, maneb, mebenil, mecarbinzid, mefenoxam, mefentrifluconazole, mepanipyrim, mepronil, meptyldinocap, metalaxyl, metalaxyl-M (=mefenoxam), metam, metazoxolon, metconazole, methasulfocarb, methfuroxam, methyl bromide, methyl isothiocyanate, methylmercury benzoate, methylmercury dicyandiamide, methylmercury pentachlorophenoxide, metiram, metominostrobin, metrafenone, metsulfovax, metyltetraprole, milneb, myclobutanil, myclozolin, N-(ethylmercury)-p-toluenesulfonanilide, nabam, naftifine, natamycin, neem oil, nitrothal-isopropyl, nuarimol, octhilinone, ofurace, organic oils, orthophenyl phenol, orysastrobin, oxadixyl, oxathiapiprolin, oxazosulfyl, oxine copper, oxolinic acid, oxpoconazole, oxycarboxin, oxytetracycline, PCNB, pefurazoate, penconazole, pencycuron, penflufen, pentachlorophenol, penthiopyrad, phenamacril, phenylmercuriurea, phenylmercury acetate, phenylmercury chloride, phenylmercury nitrate, phosdiphen, Phosphite, phosphorous acid and salts, phosphorus acid, phthalide, picarbutrazox, picolinamides, picoxystrobin, piperalin, plant oils (mixutures): eugenol, geraniol, thymol, polycarbamate, polyoxin-D, potassium azide, potassium bicarbonate, potassium polysulfide, potassium thiocyanate, probenazole, prochloraz, procymidone, propamocarb, propiconazole, propineb, proquinazid, prothiocarb, prothioconazole, Pseudomonas chlororaphis strain AFS009, Pseudomonas syringae ESC-10, pydiflumetofen, pyracarbolid, pyraclostrobin, pyrametostrobin, pyraoxystrobin, pyrapropoyne, pyraziflumid, pyrazophos, pyribencarb, pyributicarb, pyridachlometyl, pyridinitril, pyrifenox, pyrimethanil, pyrimorph, pyriofenone, pyrisoxazole, pyroquilon, pyroxychlor, pyroxyfur, quinacetol, quinazamid, quinconazole, quinofumelin, quinomethionate, quinoxyfen, quintozene, rabenzazole, Reynoutria sachalinensis, salicylanilide, sec-butylamine, sedaxane, sesame oil, silthiofam, silver, simeconazole, SJC17, sodium azide, sodium bicarbonate, sodium hypochlorite, sodium orthophenylphenoxide, sodium pentachlorophenoxide, sodium polysulfide, spiroxamine, Streptomyces griseovirides strain K61, Streptomyces lydicus strain WYEC108, streptomycin, sulfur, sulfuryl fluoride, sultropen, tea tree oil, tebuconazole, tebufloquin, tecloftalam, tecnazene, tecoram, tetraconazole, thiabendazole, thiadifluor, thicyofen, thifluzamide, thiochlorfenphim, thiophanate, thiophanate-methyl, thioquinox, thiram, THQ25, tiadinil, tioxymid, tolclofos-methyl, tolfenpyrad, tolprocarb, tolylfluanid, tolylmercury acetate, triadimefon, triadimenol, triamiphos, triarimol, triazbutil, triazoxide, trichlamide, Trichoderma afroharzianum (formerly harzianum) strain T-22, Trichoderma asperellum (formerly harzianum) strain ICC012, strain T25, strain TV1, Trichoderma asperellum strain T34, Trichoderma atrobrunneum (formerly harzianum) strain ITEM908, Trichoderma atroviride (formerly harzianum) strain IMI206040, strain T11, Trichoderma atroviride strain I-1237, Trichoderma atroviride strain LU132, Trichoderma atroviride strain SC1, Trichoderma gamsii (formerly viride) strain ICC080, triclopyricarb, tricyclazole, tridemorph, trifloxystrobin, triflumizole, triforine, triticonazole, UCQ09, Ulocladium oudemansii strain U3, uniconazole-P, urea, validamycin, valifenalate, vinclozolin, voriconazole, WLR08, zarilamid, zinc naphthenate, zinc thiazole, zineb, ziram, zoxamide, preferably azoxystrobin, trifloxystrobin, benomyl, carbendazim, thiabendazole, tebuconazole, imazalil, penconazole, procymidone, vinclozolin, mancozeb, ziram, copper oxychloride, sulfur, fludioxonil and/or iprovalicarb.

The phytosanitary composition according to the present invention can be prepared according to various processes known to a person skilled in the art, for example by a method chosen among spray dry, extrusion, fluidized-bed granulation with dehydration, freeze-drying, emulsion process, suspension in mixture with one or more chemical compounds and/or biological agents and/or with one or more co-formulants, described before.

It is a further object of the present invention a kit comprising:
a) the PT22AV yeast strain of Papiliotrema terrestris species as defined above, or a composition as defined above, and
b) one or more chemical compounds, for example synthetic chemical compounds, and/or one or more biological agents, for example microorganisms such as bacteria, yeasts, fungi, viruses, algae, or compositions of them. Both chemical compounds and biological agents can be selected among fungicides, insecticides, fertilizers, biostimulants, macronutrients, micronutrients, elicitors, phyto-regulators, microbial biological control agents, plant growth regulators, foliar nutrients, antibiotics, herbicides, acaricides, food additives, antioxidants, adjuvants, microorganisms of agri-food interest.

In particular:
said foliar nutrients can be selected from nitrogen, phosphorus, potassium, calcium, sulfur, magnesium;
   aforementioned micronutrients can be selected from iron, boron, chlorine, manganese, zinc, copper, molybdenum and nickel;
said insecticides can be selected from inorganic compounds, oils, botanical extracts, organochlorines, organophosphates, carbamates, neonicotinoids, avermectins, organosulfurs, organotins, synthetic pyrethroids;
said elicitors can be chosen from chemical synthesis elicitors, such as salicylic acid, methyl salicylate, benzothiadiazole, benzoic acid and chitosan, or elicitors consisting of plant extracts, such as tannins, citrus oils, bioflavonoids, such as dihydroquercetin, arabinba galactane, polysaccharides and others plant extracts;
said plant growth regulators can be chosen from (international nomenclature): auxins, gibberellins, cytokinins, ethilene, abscissic acid, ancymidol; chlormequat; chloro IPC; daminozide; flurprimidol; hydrogen cyanamide (H2CN2); mefluidide; mepiquat chlorure; paclobutrozol; proexadione calcium; succinic acid (SADH), forchlorfenuron.

As mentioned above, fungicides can belong, for example, to strobirulins, benzimidazoles, sterol inhibitors, dicarboxamides, dithiocarbamates, inorganic fungicides, phenylpyrroles or their combinations, and to other chemical groups.

According to the kit of the present invention, said fungicides (chemical and/or biological) can be selected from (international nomenclature) 2-methoxyethylmercury chloride, 2-phenylphenol, 3-ethoxypropyl mercury bromide, 8-hydroxyquinoline sulfate, 8-phenylmercurioxyquinoline, acibenzolar, acibenzolar-S-methyl, acylamino acid fungicides, acypetacs, Adavelt, Agrobacterium radiobacter K84, aldimorph, allyl alcohol, ametoctradin, aminopyrifen, amisulbrom, ampropylfos, anilazine, aureofungin, azaconazole, azithiram, azoxystrobin, Bacillus amyloliquefaciens (formerly subtilis) strain QST713, Bacillus amyloliquefaciens strain AH2, strain IT-45, strain FZB24, strain MBI600, strain D747, Bacillus mycoides isolate J, Bacillus nakamurai strain F727, Bacillus pumilus strain QST 2808, Bacillus subtilis var. amyloliquefaciens strain FZB24, Bacillus subtilis strain AFS032321, strain GB03, strain IAB/BS03, barium polysulfide, benalaxyl-M (=kiralaxyl), benodanil, benomyl, benquinox, bentaluron, benthiavalicarb, Benthiavalicarb-isopropyl, benzalkonium chloride, benzamacril, benzamide fungicides, benzamorf, benzohydroxamic acid, benzovindiflupyr, bethoxazin, binapacryl, biphenyl, bitertanol, bithionol, bixafen, blasticidin-S, Bordeaux mixture, boric acid, boscalid, bromuconazole, bupirimate, Burgundy mixture, buthiobate, calcium polysulfide, captafol, captan, carbamorph, carbendazim, carboxin, carpropamid, carvone, cell walls of Saccharomyces cerevisiae strain LAS117, Cheshunt mixture, chinomethionat, chlobenthiazone, chloraniformethan, chloranil, chlorfenazole, chlorodinitronaphthalene, chloroneb, chloropicrin, Chlorothalonil, chlorquinox, chlozolinate, ciclopirox, climbazole, Clonostachys rosea strain CR-7, Coniothyrium minitans, copper hydroxide, copper naphthenate, copper oleate, copper oxychloride, copper soap, copper sulfate, basic, copper zinc chromate, copper(II) acetate, copper(II) carbonate, basic, copper(II) sulfate, coumoxystrobin, cufraneb, cuprobam, cuprous oxide, cyazofamid, cyclafuramid, cyclobutrifluram, cycloheximide, cyflufenamid, cymoxanil, cypendazole, cyproconazole, cyprodinil, Cyprofuram, dazomet, DBCP, debacarb, decafentin, dehydroacetic acid, dichlobentiazox, dichlofluanid, dichlone, dichlorophen, dichlorophenyl, dichlozoline, diclobutrazol, diclocymet, diclomezine, dicloran, diethofencarb, diethyl pyrocarbonate, difenoconazole, diflumetorim, dimethachlone, dimethirimol, dimethomorph, dimoxystrobin, diniconazole, diniconazole-M, dinobuton, dinocap, dinocap-4, dinocap-6, dinocton, dinopenton, dinosulfon, dinoterbon, diphenylamine, Dipymetitrone, dipyrithione, disulfiram, ditalimfos, dithianon, DNOC, dodemorph, dodicin, dodine, donatodine, drazoxolon, edifenphos, enestrobin, enestroburin, enoxastrobin, epoxiconazole, etaconazole, etem, ethaboxam, ethirimol, ethoxyquin, ethylene oxide, ethylmercury 2,3-dihydroxypropyl mercaptide, ethylmercury acetate, ethylmercury bromide, ethylmercury chloride, ethylmercury phosphate, etridiazole, extract from Melaleuca alternifolia (tea tree), extract from Reynoutria sachalinensis (giant knotweed), extract from the cotyledons of lupine plantlets (“BLAD”), extract of Swinglea glutinosa, F500, famoxadone, fenamidone, fenaminosulf, fenaminstrobin, fenamistrobin, fenapanil, fenarimol, fenazaquin, fenbuconazole, fenfuram, fenhexamid, fenitropan, fenoxanil, fenpiclonil, fenpicoxamid, fenpropidin, fenpropimorph, fenpyrazamine, fentin, fentin acetate, fentin chloride, fentin hydroxide, ferbam, ferimzone, Florylpicoxamid, fluazaindolizine, fluazinam, fludioxonil, flufenoxystrobin, fluindapyr, flumorph, fluopicolide, fluopimomide, fluopyram, fluoroimide, fluotrimazole, fluoxapiprolin, fluoxastrobin, fluquinconazole, flurozolamide, flusilazole, flusulfamide, Flutianil, flutolanil, flutriafol, fluxapyroxad, folpet, fosetyl, fosetyl-Al, fthalide (phthalide), fuberidazole, furalaxyl, furametpyr, furcarbanil, furfural, furmecyclox, furophanate, Gliocladium catenulatum J1446, Gliocladium virens GL-21, glyodin, griseofulvin, guazatine, halacrinate, hexachlorobenzene, hexachlorophene, hexaconazole, hexylthiofos, hymexazol, imazalil, imibenconazole, iminoctadine, Inatreq (fenpicoxamid), inorganic oils, inpyrfluxam, iodocarb, ipconazole, Ipflufenoquin, iprobenfos, iprodione, iprovalicarb, isofetamid, isoflucypram, isopropanol azole, isoprothiolane, isopyrazam, isotianil, isovaledione, Jun Si Qi, kasugamycin, kresoxim-methyl, laminarin, Lime sulfur (lime sulphur), mancopper, mancozeb, mandestrobin, mandipropamid, maneb, mebenil, mecarbinzid, mefenoxam, mefentrifluconazole, mepanipyrim, mepronil, meptyldinocap, metalaxyl, metalaxyl-M (=mefenoxam), metam, metazoxolon, metconazole, methasulfocarb, methfuroxam, methyl bromide, methyl isothiocyanate, methylmercury benzoate, methylmercury dicyandiamide, methylmercury pentachlorophenoxide, metiram, metominostrobin, metrafenone, metsulfovax, metyltetraprole, milneb, myclobutanil, myclozolin, N-(ethylmercury)-p-toluenesulfonanilide, nabam, naftifine, natamycin, neem oil, nitrothal-isopropyl, nuarimol, octhilinone, ofurace, organic oils, orthophenyl phenol, orysastrobin, oxadixyl, oxathiapiprolin, oxazosulfyl, oxine copper, oxolinic acid, oxpoconazole, oxycarboxin, oxytetracycline, PCNB, pefurazoate, penconazole, pencycuron, penflufen, pentachlorophenol, penthiopyrad, phenamacril, phenylmercuriurea, phenylmercury acetate, phenylmercury chloride, phenylmercury nitrate, phosdiphen, Phosphite, phosphorous acid and salts, phosphorus acid, phthalide, picarbutrazox, picolinamides, picoxystrobin, piperalin, plant oils (mixutures): eugenol, geraniol, thymol, polycarbamate, polyoxin-D, potassium azide, potassium bicarbonate, potassium polysulfide, potassium thiocyanate, probenazole, prochloraz, procymidone, propamocarb, propiconazole, propineb, proquinazid, prothiocarb, prothioconazole, Pseudomonas chlororaphis strain AFS009, Pseudomonas syringae ESC-10, pydiflumetofen, pyracarbolid, pyraclostrobin, pyrametostrobin, pyraoxystrobin, pyrapropoyne, pyraziflumid, pyrazophos, pyribencarb, pyributicarb, pyridachlometyl, pyridinitril, pyrifenox, pyrimethanil, pyrimorph, pyriofenone, pyrisoxazole, pyroquilon, pyroxychlor, pyroxyfur, quinacetol, quinazamid, quinconazole, quinofumelin, quinomethionate, quinoxyfen, quintozene, rabenzazole, Reynoutria sachalinensis, salicylanilide, sec-butylamine, sedaxane, sesame oil, silthiofam, silver, simeconazole, SJC17, sodium azide, sodium bicarbonate, sodium hypochlorite, sodium orthophenylphenoxide, sodium pentachlorophenoxide, sodium polysulfide, spiroxamine, Streptomyces griseovirides strain K61, Streptomyces lydicus strain WYEC108, streptomycin, sulfur, sulfuryl fluoride, sultropen, tea tree oil, tebuconazole, tebufloquin, tecloftalam, tecnazene, tecoram, tetraconazole, thiabendazole, thiadifluor, thicyofen, thifluzamide, thiochlorfenphim, thiophanate, thiophanate-methyl, thioquinox, thiram, THQ25, tiadinil, tioxymid, tolclofos-methyl, tolfenpyrad, tolprocarb, tolylfluanid, tolylmercury acetate, triadimefon, triadimenol, triamiphos, triarimol, triazbutil, triazoxide, trichlamide, Trichoderma afroharzianum (formerly harzianum) strain T-22, Trichoderma asperellum (formerly harzianum) strain ICC012, strain T25, strain TV1, Trichoderma asperellum strain T34, Trichoderma atrobrunneum (formerly harzianum) strain ITEM908, Trichoderma atroviride (formerly harzianum) strain IMI206040, strain T11, Trichoderma atroviride strain I-1237, Trichoderma atroviride strain LU132, Trichoderma atroviride strain SC1, Trichoderma gamsii (formerly viride) strain ICC080, triclopyricarb, tricyclazole, tridemorph, trifloxystrobin, triflumizole, triforine, triticonazole, UCQ09, Ulocladium oudemansii strain U3, uniconazole-P, urea, validamycin, valifenalate, vinclozolin, voriconazole, WLR08, zarilamid, zinc naphthenate, zinc thiazole, zineb, ziram, zoxamide, preferably azoxystrobin, trifloxystrobin, benomyl, carbendazim, thiabendazole, tebuconazole, imazalil, penconazole, procymidone, vinclozolin, mancozeb, ziram, copper oxychloride, sulfur, fludioxonil and/or iprovalicarb.

The present invention also concerns the use of the PT22AV yeast strain as defined above, of the extracellular substances secreted by said strain, of a composition or of a kit as defined above against phytopathogens of one or more plants o crops, for example agricultural crops, ornamental crops, forest crops, wild species, industrial crops, plants in parks and gardens. According to the present invention, said yeast strain, extracellular substances, composition or kit can be used before sowing, pre-harvest or post-harvest.

In particular, according to the present invention, the yeast strain PT22AV belonging to Papiliotrema terrestris species is effective in the control of phytopathogens, since it acts by controlling their development and so limiting the establishment of infections in plant, without directly killing the phytopathogen. Therefore, the term “against phytopathogens” mentioned above is meant for the control of phytopathogens.

The extracellular substances (or secondary metabolites) secreted by the PT22AV yeast strain, belonging to Papiliotrema terrestris species, during the biomass production phase, are essentially polymers of a glucidic nature, the most frequent monomer units of which are: mannose; xylose; glucose, galactose, arabinose. The structure of these polymers is organized in main chains of monomers linked through linear and branches bonds. Therefore, the present invention includes the use of wastewater, (i.e. cultural growth broth applied as it is (crude) or concentrate, with high content of EPSs (extracellular polysaccharides) compounds) from the Papiliotrema terrestris PT22AV biomass production process against phytopathogens, i.e. as plant protection agents or biocontrol agents against plant pathogens.

In particular, according to the present invention, the yeast strain can be used in the direct and indirect control of all harmful organisms for plants. For example, the strain can be used for the induction of resistance and biostimulation of plants, improving the tolerance of plants to pathogens, including viruses, thanks to the production of polysaccharides and other metabolites. This highly complex matrix protects the strain from abiotic stressors, improves adhesion to surfaces and triggers an induction of plant resistance thus improving tolerance to a wide range of pathogens, including viruses.

Liquid or dehydrated preparations based on extracellular polymers, of protein and carbohydrate nature, or secondary metabolites, which accumulate during the fermentation and subsequent industrial transformation of the PT22AV strain, can be used as phytostimulants, inducers of resistance against pathogens, abiotic stress relievers (osmotic, water, thermal, UV, etc.), healing films, adjuvants in the distribution of foliar applications or seed coating, growth medium for microorganisms, including Bio Packaging (integration or replacement of Packaging to preserve food products, etc).

According to the use of the present invention, said one or more plants or crops can be selected from cereals, such as wheat, barley, rye, oats, rice, corn, sorghum; fruit trees, such as olive, apple, pear, apricot, nashi, plum, peach, almond, cherry, persimmon, banana, vine, strawberry, raspberry, blackberry trees; citrus fruits, such as oranges, lemons, mandarins, clementine trees, grapefruits; legumes, such as beans, peas, lentils, soy; vegetable crops, such as spinach, lettuce, asparagus, artichokes, cabbage, carrots, onions, garlic, tomatoes, potatoes, aubergines, peppers, fennel; cucurbits, such as squashes, zucchini, watermelons, melons; oil plants, such as soy, sunflower, canola, peanut, castor, coconut; tobacco; coffee; tea trees; cocoa; sugar beet; sugar cane; cotton.

According to the present invention, said phytopathogens can be fungi, such as for example Basidiomycetes, Ascomycetes, Deuteromycetes, Oomycetes, fungi with sexual or asexual reproduction, fungi with biotrophic or necrotrophic activity, protists or Chromista. For example, the strain can be used for the control of mycotoxigenic fungi that affect plant production both in the field and in the post-harvest stage, preventing the accumulation of mycotoxins on any agricultural crop.

In particular, according to the present invention, said fungi can be selected among the species Albugo spp.; Alternaria spp.; Anthracnose; Armillaria spp.; Ascochyta spp.; Aspergillus spp.; Blumeria graminis; Botrytis cinerea; Botrytis spp.; Bremia lactucae; Cercospora kikuchii; Cercospora sojina; Cercospora spp.; Cercosporella herpotrichoides; Cladosporium spp.; Claviceps purpurea; Colletotrichum spp.; Corynespora cassiicola; Diaporthe spp.; Erysiphe spp.; Fusarium graminearum; Fusarium oxysporum; Fusarium spp.; Helminthosporium spp.; Leveillula Taurica; Macrophomina phaseolina; Magnaporthe oryzae; Magnaporthe spp.; Melampsora lini; Monilinia spp.; Mucor spp.; Mycosphaerella graminicola; Mycosphaerella spp.; Oidium spp; Penicillium spp.; Peronospora manshurica; Peronospora spp.; Phaeosphaeria spp.; Phakopsora pachyrhizi; Phakopsora spp; Phoma spp.; Phytophthora spp.; Plasmopara viticola; Podosphaera spp.; Pseudopernospoea cubensis; Puccinia spp.; Pyrenochaeta lycopersici; Pyrenophora spp.; Pyricularia oryzae; Pythium spp.; Ramularia spp; Rhizoctonia solani; Rhizoctonia spp.; Rhizopus spp.; Rhynchosporium spp.; Sclerotinia sclerotiorum; Sclerotinia spp.; Sclerotium cepivorum; Sclerotium rolfsii; Sclerotium spp.; Septoria glycines; Septoria spp.; Sphaerotheca spp.; Stemphylium spp.; Stenocarpella maydis; Thielaviopsis basicola; Thielaviopsis spp.; Tilletia spp.; Uncinula spp.; Uromyces spp.; Ustilago maydis; Ustilago spp.; Venturia spp.; Verticillium spp.

Furthermore, according to the use of the present invention, said phytopathogens can be bacteria, viruses or parasitic nematodes of crops. In particular, the bacteria can be selected from among the species Pseudomonas spp., Xanthomonas spp., Clavibacter spp., Ralstonia spp., Erwinia spp, while nematodes can be chosen among the species Meloidogyne spp., Heterodera spp., Globodera spp., Belonolaimus spp., Pratylenchus spp., Rotylenchulus spp., Trichodorus spp., Paratylenchus spp. etc.

According to the use of the present invention, said yeast strain or said extracellular substances or said composition or said kit can be applied on one or more parts, intact or wounded, of the plant selected from seeds, aerial parts, leaves, stems, trunks, buds, gems, branches, stems, flowers, fruits, roots.

In particular, for field treatments on plants aerial part, the PT22AV strain, or the extracellular substances, can be applied using the distribution equipment currently in use or the aerial irrigation system, for example by means of spraying equipment or overhead irrigation systems. Treatments on fruit, flowers and fresh-cut produce during the post-harvest stage can be applied using distribution equipment or added to the washing water. According to the present invention, the quantity of living cells of said yeast strain distributed per unit of culture surface can range from 10⁸ to 10¹⁵ CFU per hectare.

According to the present invention the dosage of PT22AV-based compositions can vary according to the type of formulate and the field of application, generally from a few grams to tens of kilos per hectare. The dosage can also depend on the crop, the pathogen to be countered, the pressure of the disease, the weather and other agronomic factors. According to the use of the present invention, aforementioned yeast strain or its extracellular substances or the composition or the kit, as described above, can be applied to solid plant growth substrates, such as soil, preferably at a live cell concentration of the yeast strain ranging from 10² to 10¹² CFU per gram of solid growth substrate (soil or another matrix).

According to the present invention, soil treatment can be carried out by applying the formulate based on the PT22AV strain of Papiliotrema terrestris, by dilution in the substrate, sprayed or distributed with a fertigation system. Soil treatment can be aimed at improving the chemical-physical and microbiological characteristics of the soil and/or for the control of phytopathogenic nematodes.

Moreover, according to the present invention, PT22AV strain can also be used for the treatment of a hydroponic or floating crop system, for example against pathogens and root parasites; in this case the product can be applied directly in the nutrient solution.

Furthermore, said yeast strain or said composition can be used as seed coating agents; in this case the strain concentration can range from 10² to 10¹⁰ CFU per gram of seed. According to the invention, PT22AV strain used as seed treatment can be applied in combination with filming, surfactant or tackifying agents.

In addition, said yeast strain or said composition can be used by means of pollinating insects to counteract plant pathogens capable of penetrating the host through the flower (Erwinia spp., Pseudomonas syringae, Botrytis cinerea, etc.).

According to the use of the present invention, said phytopathogen can be chosen from Botrytis cinerea and Plasmopara viticola of vine; Botrytis cinerea of strawberry, Botrytis cinerea and Phytopthora infestans of tomato, Venturia inequalis and Stemphylium spp. of apple and/or pear tree, Monilia spp. of stone fruit and/or Phytophtora spp of potato. According to the present invention, PT22AV yeast strain, its extracellular substances, its composition or kit, as described above, can also be applied to the seed and their use can take place in green-house, in the field or in post-harvest stage, in a biological or integrated regime. Furthermore, when the kit as defined above is used, said components a) and b) can be used separately or sequentially.

“Separate use” is intended as the simultaneous application of components a) and b) of the kit in distinct forms. “Sequential use” is intended as the subsequent application, in any order, of components a) and b) of the kit, each in a distinct form.

The present invention also relates to a method for the control of phytopathogens in agricultural crops comprising or consisting in applying an effective dose of the PT22AV strain, of the extracellular substances secreted by it, of a composition or a kit as defined above on said crops or on the soil where the crop grows, before sowing, pre-harvest or post-harvest. The phytopatogens and the crops according to the method of the present invention can be those described above.

According to the method of the present invention, said application can take place on one or more plant parts, intact or wounded, selected from seeds, aerial parts, leaves, stems, trunks, buds, shoots, branches, stems, flowers, fruits, roots.

Furthermore, according to the method of the invention, the dose of aforementioned composition varies from 10⁸ CFU to 10¹⁵ CFU per hectare. Some examples of application protocols, with reference to the dosage, the volume of application, the method of application and the site of application of Papiliotrema terrestris PT22AV, in the main crops of agricultural interest and against the main crops' pathogens, are shown in Table 2.

TABLE 2
Application details of formulation based on PT22AV

| | | | Application rate per treatment | | | |
|---|---|---|---|---|---|---|
| | Pests or | | Active | | | |
| | Group of pests | Growth stage & | substance/ha, Kg, | Water L/ha | Application | |
| Crop | controlled | season | mL | min/max | Method | Site |
| Tomato | Botrytis | Till BBCH 63 | 0.5-1.5 × 10¹² | 500-1000 | Spray | Foliar |
| | cinerea | every 7-10 days | per ha | Taking into account | | |
| | Phytophthora | depending on the | | the vegetation | | |
| | infestans | pressure | | | | |
| | Soil diseases & | Seed coating | 0.5-1.5 × 10¹² | — | Spray | Seed coating |
| | nematodes | | per Kg seed | | | |
| | | Seedling (radical pre- | 1-5 × 10⁷ | | Suspension | Root soaking |
| | | transplant application) | per mL | | | |
| | | Transplant | 1-5 × 10⁶ | | | Fertigation |
| | | (fertigation) | per mL | | | |
| | | Fertigation | 1-5 × 10⁶ | | | |
| | | (applications following | per mL | | | |
| | | transplantation) | | | | |
| Grape | Botrytis | Till BBCH 67 | 0.5-1.5 × 10¹² | 500-800 | Spray | Foliar |
| (Wine & | cinerea | every 7-10 days | per ha | Taking into account | | |
| Table) | Oidium | depending on the | | the vegetation | | |
| | tuckeri | pressure | | | | |
| Strawberry | Botrytis | Till BBCH 65 | 0.5-1.5 × 10¹² | 400-600 | Spray | Foliar |
| | cinerea | every 5-10 days | per ha | Taking into account | | |
| | | depending on the | | the vegetation | | |
| | | pressure | | | | |
| Stone fruit | Monilinia spp. | Till BBCH 69 | 0.5-1.5 × 10¹² | 800-1000 | Spray | Foliar |
| | | every 10-15 | per ha | Taking into account | | |
| | | days depending on | | the vegetation | | |
| | | the pressure | | | | |
| Pome fruit | Stemphyllum spp. | Till BBCH 79 | 0.5-1.5 × 10¹² | 800-1000 | Spray | Foliar |
| | | every 10-15 days | per ha | Taking into account | | |
| | | depending on the | | the vegetation | | |
| | | pressure | | | | |
| | Post-harvest | Post-harvest | 5 × 10⁶-5 × 10⁷⁻ | — | Suspension in | Fruit |
| | diseases | | per mL | | washing water | |
| Wheat | Blumeria | First application | 0.5-1.5 × 10¹² | 400-600 | Spray | Foliar |
| | graminis | BBCH 39-42 | per ha | Taking into account | | |
| | Septorio tritici | Second application | | the vegetation | | |
| | Fusarium spp. | BBCH 51-53 | | | | |
| | Puccinia spp. | | | | | |
| Potato | Phytophthora | Till BBCH 66 | 0.5-1.5 × 10¹² | 500-800 | Spray | Foliar |
| | infestans. | every 5-10 | per ha | Taking into account | | |
| | | days depending on | | the vegetation | | |
| | | the pressure | | | | |
| Ornamentals | Foliar | AR phenological | 5 × 10⁶-5 × 10⁷⁻ | — | Spray | Foliar |
| | pathogens | stages with intervals | per mL | | | |
| | | of 5-15 days | | | | |
| | | depending on the | | | | |
| | | pressure of the | | | | |
| | | disease | | | | |
| Turf | Fungal | AB phenological | 0.5-1.5 × 10¹² | 500-1000 | Spray | Foliar |
| | pathogens | stages with intervals | per ha | Taking into account | | |
| | | of 5-15 days | | the vegetation | | |
| | | depending on the | | | | |
| | | pressure of the | | | | |
| | | disease | | | | |
| Plant | — | Application to | 0.5-1.5 × 10¹² | In fertigation | Drip | Foliar |
| Biostimulant | | soil/root during all | per ha | | irrigation | |
| | | phenological stages | | | | |
| | | with intervals of 5-10 | | | | |
| | | days | | | | |

The dosages and volumes of distribution are exemplary and can be varied according to the disease pressure and the type of vegetation. The present invention will now be described, for illustrative but not limitative purposes, with particular reference to the examples and attached figures, in which:

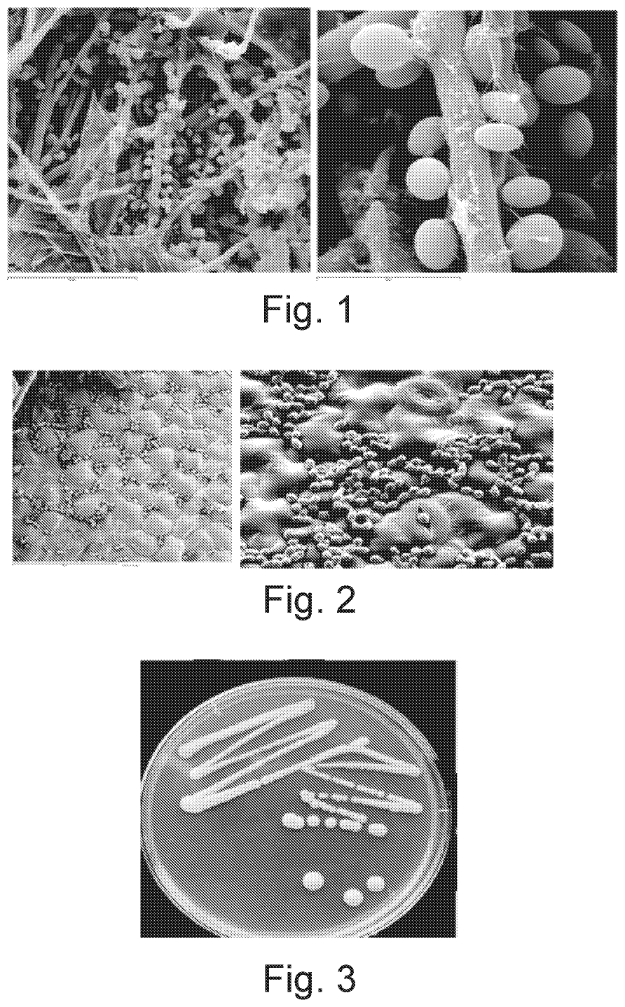

FIG. 1 shows the colonization of P. expansum hyphae by the biocontrol agent PT22AV. Image of the in vivo interaction between P. expansum and P. terrestris strain PT22AV observed under a scanning electron microscope. The yeast colonizes the fungal hyphae and adheres to them by means of an extracellular matrix of a polysaccharide nature;

FIG. 2 shows foliar colonization by the PT22AV strain 5 days after the application. The image shows the yeast-leaf interaction 5 days after application observed under a scanning electron microscope, in particular the yeast on the upper and lower page of tomato leaves, where colonization of a stoma, natural opening, is also shown;

FIG. 3 shows the PT22AV yeast grown on an agarized growth medium after 3 days of incubation at 28° C.;

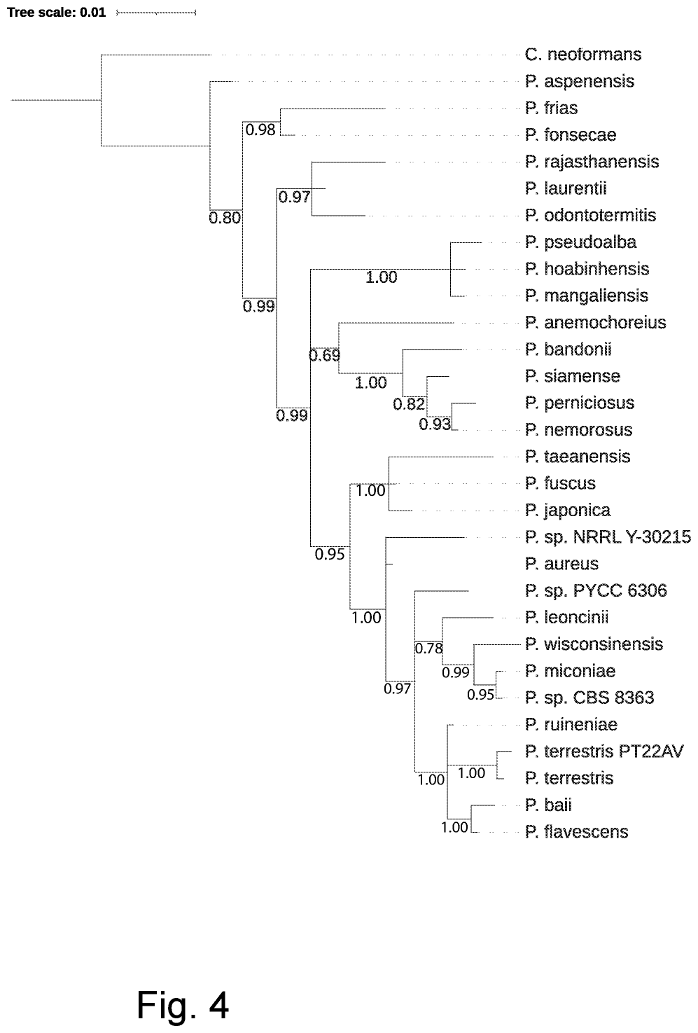

FIG. 4 shows the phylogenetic tree of the PT22AV strain obtained by analyzing the nucleotide sequence of the large subunit ribosomal RNA gene and comparing it with that of other related species;

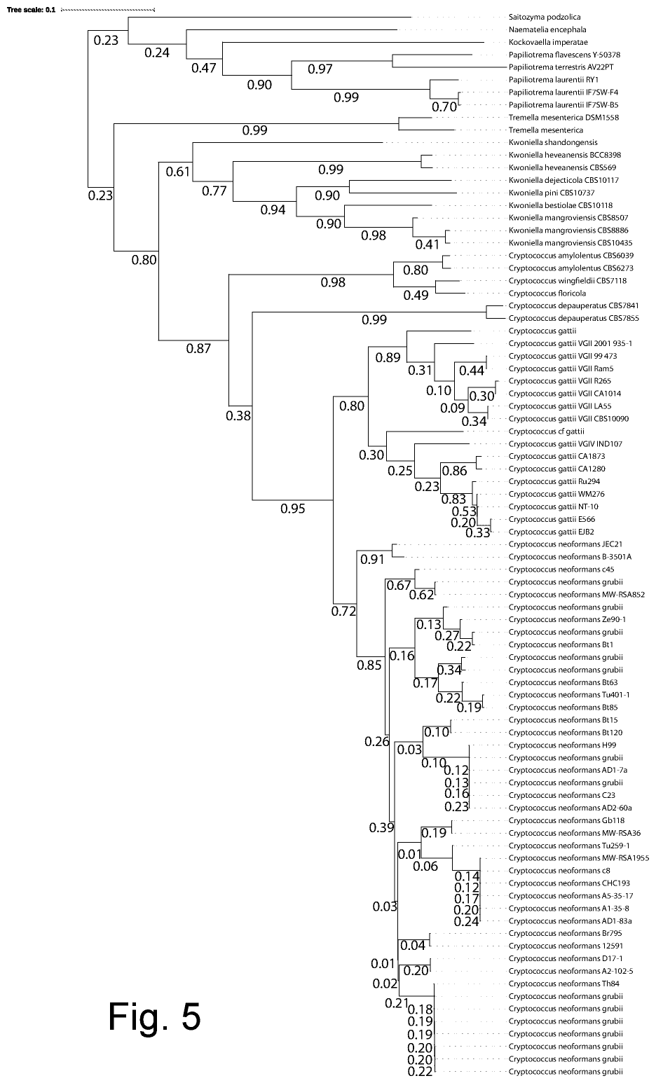

FIG. 5 shows the phylogenetic tree generated by comparing the entire proteome of the PT22AV strain with that of other species of basidiomycete's yeast.

### EXAMPLE 1. ISOLATION AND CHARACTERIZATION OF THE PT22AV STRAIN OF PAPILIOTREMA TERRESTRIS

The PT22AV strain was selected from a yeast collection isolated from the epiphytic microbial community of various fruits and vegetables in central Italy.

The PT22AV strain was identified by sequencing the large subunit ribosomal RNA gene, clearly highlighting belonging to the Papiliotrema terrestris species (FIG. 4).

In particular, the nucleotide sequence of the large subunit ribosomal RNA gene region of the PT22AV strain is shown below:

(SEQ ID NO: 1)
CCGGGAAGAGCTCAAATTTGAAATCTGGCGTGCTCAGTGCGTC
CGAGTTGTAATCTATAGAGGCGTTTTCCGTGCCGGACTGTGTCTAAG
TCCCTTGGAACAGGGTATCAAAGAGGGTGATAATCCCGCACTTGACA
CAATGACCGGTGCTCTGTGATACGTCTTCTACGAGTCGAGTTGTTTG
GGAATGCAGCTCAAAATGGGTGGTGAGTTCCATCTAAAGCTAAATATT
GGCGAGAGACCGATAGCGAACAAGTACCGTGAGGGAAAGATGAAAA
GCACTTTGGAAAGAGAGTTAAACAGTACGTGAAATTGTTGAAAGGGA
AACGATTGAAGTCAGTCGTGACTGAGAGGCTCAGCCGGTTCTGCCG
GTGTATTCCCTCAGTCGGGTCAACATCAGTTTTGTTCGGTGGATAAG
GGCAGCTGGAAGGTGGCACCTCCGGGTGTGTTATAGCCAGCTGTCG
CATACATCGAATGAGACTGAGGAATGCAGCTCGCCTTTATGGCGGGG
TCGCCCACGTCGAG.

The Applicant obtained the entire genomic sequence of the PT22AV BCA strain by Nanopore sequencing technology. The bioinformatic analysis of the sequence of the entire PT22AV genome allowed the prediction of the entire proteome, also used for the taxonomic analysis of the strain, hence precisely defining the differences with other strains of related species (FIG. 5).

The entire mitochondrial genome of the PT22AV strain consists of the following sequence:

(SEQ ID NO: 2)
CTATGAGTATAGCACTAGGAAGCTAACCATTAGGGCGAATAGCCCTG
TAGCTTCGGCTAGAGCGAATCCTAGAATAGCGTATGTGAATAGTTGT
CCTCGAACAGCAGGGTTTCGAGCTGTACCAGCGATTAGAGCGCTGAA
TACTGTACCGATCCCAACACCGGCTCCTGAGAGACCGATAGCGGCTA
GACCAGCACCAATGAACTTTGCAGCAGCCATTGTGTATTGTGATTGTC
TGAACCNNNNNNNNNNTCAATGAATTGAGCTTTAGCCTTTATAATCTA
AAGGCTAGTGATGACATCCATGAACGGCTATCACCTGTATAAAGACA
GTGATAGAATATATGTGATCATGGAGGGGACAGACGTGGATCTCTAT
TATGAACTCTATAGTGCGGTAGCTGGATCTCAGACTATATTACCGTAT
CTACCCTTTTAGGACCTATAATCATGCTTATAATATCTATAGATGGTAG
ATTACCTAACACTCGGTGCTATAGTAGGTTATAGGAGTATTATACTCC
TATAACCATAAACTATGAATGGCTACTATCAGCATGAGCAATGACTCT
ATTTACCGCTATAGTAATGGTGTACTCTACCATTTTTCCTATAGGATCC
TACCTATCCCTATAAGCTATAGGGTTTTCTATGAAAATGCTCTACAATA
CATGTATGCTAACGTGAGTCAATGAGTATAGCCTCGGAAACTTTATTT
ATGCACTTCTGACGCCATGCTGAATATAATAGCGCCACATGTATGCAT
AGCTTTTATCTCTCTCTATACAAGTTATTATTTATTAGTAAATTTATTAC
TAAATTTATTTTTAATAAATAAATTATTATTATTATTATATTGGTATGGA
CGGACAGAGCAAGATTCGAACTTGCGGTGGGCGGGACCCACTTTGAG
GTTCAAGCTCAATGCCTTAGACCACTAGGCTATCTGTCCTGTTATCTC
ATCGACTCACCTGCTACCCTTAAAGAGGGGTAGGAAAAATGGTGAAA
TATAGTATATTGTCTATCTATAAACTTTTATAAGAATAGTTACGGTCAT
AGATTTTTATCGTCTCGTCTCTACACAATGGCTATAACCCCTTACTATT
AAGGCCAGCCTATATTTTTTGCTGCTATAGGTGACACAGAATATTTAT
GTTAAAGAACATAAGGGGGCTAGTATAAAGTCCAAGCATAATAGCTTT
GTAACGTATACACGGCTCACAATCAGTTGAAGGATCGGAGGTAGTAC
GAACACTGATACAACGTATACCAGTGCAGCCAGAGTAAGGAGAACGA
ATGCTCCTTGGTTAAGGAAGTAAAATGGGAGAGCTTGAGGCATAGGC
TGATATGCAACAGGCAATGTGATAGGTGTTTCCATCATCACTATGAAT
ACTATAATTATGAGTGACTATATAGCATGCTCATAATAACAATAGACTC
TTACATACTTTCCTTTCNNNNNNNNNNCTTCTGACGCCATGCTGAATA
TAATAGCGCCACATGTATGCATAGCTTTTATCTCTCTCTATACAAGTTA
TTATTTATTAGTAAATTTATTACTAAATTTATTTTTAATAAATAAATTAT
TATTATTATTATATTGGTATGGACGGACAGAGCAAGATTCGAACTTGCG
GTGGGCGGGACCCACTTTGAGGTTCAAGCTCAATGCCTTAGACCACT
AGGCTATCTGTCCTGTTATCTCATCGACTCACCTGCTACCCTTAAAGA
GGGGTAGGAAAAATGGTGAAATATAGTATATTGTCTATCTATAAACTT
TTATAAGAATAGTTACGGTCATAGATTTTTATCGTCTCGTCTCTACACA
ATGGCTATAACCCCTTACTATTAAGGCCAGCCTATATTTTTGCTGCTA
TAGGTGACACAGAATATTTATGTTAAAGAACATAAGGGGGCTAGTATA
AAGTCCAAGCATAATAGCTTTGTAACGTATACACGGCTCACAATCAGT
TGAAGGATCGGAGGTAGTACGAACACTGATACAACGTATACCAGTGC
AGCCAGAGTAAGGAGAACGAATGCTCCTTGGTTAAGGAAGTAAAATG
GGAGAGCTTGAGGCATAGGCTGATATGCAACAGGCAATGTGATAGGT
GTTTCCATCATCACTATGAATACTATAATTATGAGTGACTATATAGCAT
GCTCATAATAACAATAGACTCTTACATACTTTCCTTTCTATGAGAGGTT
GGTTAGTTAGCAAGGGCAGAAGGGGACTATAGTTATTGGTAACCATA
TAAGGTCGTTATAACGTTTCAACAAGCCTATGATTGGATAGGTAGCAT
GTCGAATGCGTGGTGTGGAGTAGGTGATGGTAGTACCCACTCGATTG
TGTAGGCGAACCGTGTGCTTCGTTCGTACGTTAGGTTGTCCGTGAAG
TATAGCGGTTCTCCCCATGGGTTGTCACCAACGTACTTGTCGTTAGT
GAACATGTCGTAAACAGTGTACAGGAACAGAAGAGTAGCAGAGACGC
TAACGAATGAACCCCATGATGAGATTAGGTTCCATCCCGCGTAAGCG
TCAGGGTAGTCAGCGAATCGTCGTGGCATTCCTTGTAGTCCTAGGAA
GTGTTGTGGCATAAATGTCATGTTAACACCGATGAAGAATGTCCAGAA
GTGCGCTTGAGCTAGGTGTTCTTCGTACATCTTACCGAACATCTTTGG
AGCCCAGTAGTAGAACCCGGCAAAGATACTGAATACGGCACCCATTG
AAAGCACGTAGTGGAAGTGAGCCACAACGTAGTATGTGTCATGCATC
GCTACGTCAACGCTCGCGTTCGCTAGAACTACACCTGTCAGTCCACC
AATAGTGAACAGACCGACGAATCCTAGAGCGAATAGCATAGGTGCTA
GCAGTCGGATAGAACCACCGTAAGCAGTCGCTAGCCAACTGAAGAC
CTTGATACCTGTAGGTACCGCAATAATCATAGATGCAGCTGTGAAGTA
AGCTCGTGTGTCTACGTCCATCCCTACGGCGTACATGTGGTGGCTCC
ATACAATGAATCCAAGCACACCGATAGAGGCAATGGCGTACACCATA
CCAAGGTAACCGAAAACAGGCTTTCCACTGAATGTTGAAACAACGTG
GCTAACCATACCAAATCCAGGGATAATCATTAGGTATACTTCAGGGTG
TCCGAAGAACCAGAATAGGTGTTGGTATAGGACAGGGTCCCCTCCTC
CAGCAGGGTCGAAGAATGATGTGTTGAAGTTACGGTCAGTAAGGATC
ATAGTAAGAGCTCCGGCTAGAACTGGAATACATAGAATAATAATAACT
GACTGTGATAGCATCGCCCATACGAATAGAGGTGCCTTGTGTAGGCG
TAGCCCTGGAGCTCGCATGTTAAGGATTGTAGTGATAATGTTAATGG
CACCAAGTAGAGATGAAATCCCTGAGAGGTGCAGGCTGAATACAGCT
AGATCAACTGATCCACCACTGTGTGATTGTACACCTGTTAGAGGCAT
GTACATAGTCCAACCGCTACCCATACCTTGTTCCACGAAGACGCTTG
AAAGGATAAGAACGATTGCAGGAGGTAGTAGCCAGAAGCTGATGTTG
TTCAGCCGTGGGAACGCCATGTCCGGCGCACCGATAAGAACGGGAG
CCATGTAGTTAGCGAACCCAGCCATAGCAGGTACCACCATGAAGTAA
ATCATCATGATACCGTGAGTTGTAGCAATTACGTTGTAGAGGTGGTG
GTTACCAGAAAGGAACTGGTTACCAGGAGCTGATAGCTCCATCCGGA
TAAGTACACTGAACGCAGTACCCAGAAGACCGGTAAAGACAGCAAAA
AGGATATAAAGGGTACCAATATCCTTGGCGTTTGTAGAGAAGAGCCA
TCGGCTAGCCATGTTAATAAGAGGGTTATGCTGTTATGGGGAGGGGT
AGGTAGGTTGACAGCTGCTGGTGAGAGTAACTATAGTGCATAGCAAT
ATTCATGGTAGAGTATTCTATCACTATAGGCTCTAGTTATCTACCATAA
AAATGGACATAGCGGATAGCCATGAGAACTAAAACAGTAGAAACTGA
TAGATGCCTTAATAGGCAGAAAGTGTCACAAATGTGAGGTCTTTAATC
CACAACTAAGACTTAGAGTAAATAGAAGGGAGGAGGTTCACGGTATT
AGTAAATCATAGAGGAGAGAGAGAGATGCATCACTAGTAACCCATAG
GGTACACAGCAGTGCTAGTAGCAAGTCTTTAATAATCATATGTGATAG
ATGGCGGAATAGCCTAATCTACACACTATTAGATGTAGGTACTTTAGA
TATGATATAATTTAAAACATTTATAGCTGTAATTATAAAAACTCGCTATA
GGCTTATAATATTATAATATGACCATATATTTTATATAAAGCATAACAA
CTATGTATGGCAATATAAGTCCTATAGCCATCTAGCACTACAATGCAG
AGTGTCTAGGTATGACAGTTTAAGTAACAGCCTATATACATAACAACT
AAACCTTATAACTGTACAGGGTCTTAGTCCACCACATGTCACACACGA
GTATGGACTTCTGCTTAGGCAAAGCCCCTTTATACTAACCTTCCCATC
AGAAACCTTTATACTCAACCAAAGGGTTAATGTTAGGTTTATAGGGTA
GTTGTTATTTAAAAAATAAGGAGGGGTTATGGAGGCATGCTTTGCGA
GTATACCATAAAAACAAATAACATATAAATATGACTGCACAACAACGAT
CATTTGTCGAGGCGCTACTACGTCTACGGCCGCTATTCCAATCACAC
CCTCACCACCTTACTCTGGAATCACCATGGCCAATCCTTACAAGTGG
AGCTGTGCTAGCTATGCTTTCAAGCGCTGCCCTTTGGTTCAACGGTC
TACAATACTCAGGTACATTCCTTATCCTTGGGATGCTTAGCACACTAA
CAGCTATGATCCTATGGTGGGCTGACTGTGTTAAGGAAGGTACATAC
CTTGGACACCACACTAAGGTAGTTCAACAAAACCTTGCTATGGGTGTT
GCGCTTTTCATTGTAACAGAAGCCGCACTATTTGTGAGCATCTTCTGG
GCCTACTTCCACTCATCACTAGCACCTACAGTGGAACTAGGTACTCAA
TGGCCACCCGCTGGTATCACAGCCCTATCACCTATGGCTATCCCCCT
ACTCAATACCGTTCTACTACTTAGCAGCGGTGCTACAGTTACATGGG
GTCACCACGCATTCTTCTGCCGAAACCGATCAGCTGCTCTTCAAGGA
CTTATNNNNNNNNNNCAAGGACTTATGTTTACAGTAGCTCTAGCTATT
ATCTTCACTGCCCTACAAGGTGTTGAATACAACATGGCTGGATTTACT
ATTGCTGACGGTGCTTACGGTAGCTGTTTCTATATGGCCACAGGTGC
TCACGGACTACACGTGCTGGTAGGTACACTAGCTATCACTGTTGGTC
TTGCACGACTTATTAACTACCAACTTACATCAACTCACCACATTGGTC
TAGAAGGAGCAATCCTATACTGGCACTTTGTGGACGTCGTTTGGGTC
ATCCTATACGTTACAGTTTACTGGTGGGGAGCTCCATTATCCCTATCT
AAATTAGTCCCCTATTTATATATTTTCCCCTTCCAGAATACTACTTACC
TTGTTATACTTAACCTTTTAATTATAAGTTAATTGGCTGAGTAGAATAG
GGTTACAATAGAGGTAGATGAGGAACAAGAACACTATAGTATGCTTA
GTTACCATAGAAAATTTAAATTTTTATAAGCTATGAAGTGACTATTTCT
TAAATGGCTATCAGTTATTTAAGACAACTTGTTAGAAGTCAATAGTCAT
AATATCTAATAGCATGGCTCATTTTTATATTAAAATATTAACAGTATTTA
AAATTTAAAGTCATTTAATGGTATACTGCGTTTAGTATAAAAATAGAGG
ATTATATTGACACGTTTACCAGCCGAATTAATACAATTACTAAATTTAG
AGTGAGTCTAATTCAGGTGGGCTGGGCTCGAACCAGCGGCCTCATG
CTCCCAAAGCATACGTCTTAACCAAACTCGACTACCACCTGTGTACTC
TTAGCTACCGGCCCCATACAAACCATCATTAAAGTTGATAATTATTGA
TACTATTAACCTATTGTAGTATCTTTAATGAGGGGTCTAAACTGGTACA
ACGTCAAACGCAACAAGTACGCTTGGTACCAGTAGAAGTAGAGCGAT
AGCTGGTGGTAGCATTCCAGTCCAACAGAAAGAAAGTAGTTGATCGT
ACCGAAGTCGTGGTAGTGTAGCCCGGAACCACACAAAACCAAAACAA
CCGGCACAAGTCTTAAGCGCCAGCACTAGTGATGATAGTGACAGTGG
TGTACCATTGGCAATAACTTCAGGGAATCCCCATCCACCTAGGAATA
GTGTAGCTGATAGCGTGCTCATGAGAACGATAGAACAGTATTCTCCT
AGGAAGAAGAGTACGAAAGGCATAGCACTGTGCTCTGTCATGAACCC
AGCAACAAGTTCTGATTCAGCTTCAGGAAGGTCAAAGGGTGTTCGGT
TTGTTTCGGCTAGTACAGATACAATGAATAGCATGAATAGAGGCGCTA
GAGGAACACAGTACCAGATAGGTGCTTGTGATTCTACAATAGCCGTG
TGGTTAAGGCTACCGGCCATGATCAGGACTGCTAGAACAGCAGAACC
TAGGATCAGTTCATAGCTAATCATTTGAGCTGTTGATCGTAGCCCACC
AAGAAGAGCGTATGATTCGTTGGCAGACCATCCAGCTAGTAGTACCC
CATAAACACCAACACTAGAAACAGCTAGAGAGTATAGAACCCCTAGTT
CTAGGTCAGCAATAGCGGCACCAGGACCGAATGGCATAACAGCCCA
ACCAAGAAGGGCGAACACCAGTGTAATAACAGGTGCTAGGTAGAAGA
TGACGGTGTTAGAGGCCCCCGGAACAACAGTTTCCTTTACCACCAGC
TTCAGGGCGTCAGCGAACGGTTGTAGTACTCCATAGACACCTACTGC
GTTAGGCCCTACTCGTCGTTGCATTGCAGCCAGAACCTTACGTTCGA
TAATGGTCATGAACGCAACAGCTAGAAGAATAGGTACCAGTACAGCT
AGCACAGACAGAATGGAGAAGAGAGTAGAAAGCATATTACGTAGTTT
GTAGACATAAAACCCGTGATACTTAAAAGACTAAGACCAGTCGGGAG
GTATGGGAGAGGTACACTATAAATAATAATAATGAATGAATATATATA
GACAGACAGTACATAACTGTGCCGTATGGTTATATTTATCAATCTTAC
CTAATTGTTAAAACATGGTTATAATAACCACCCTTATAAGTATAACTAA
GGTAGTCTATAATGGTTATAGGAACCAGAATAGTCATGAATATAAGAG
CACAATGGCTCCTAGCGATACGTTAAGATAAATAGTGTGGTGATAGTT
AAATAAAGGGGTCTATAGCAATGGCAGAAATAACCATAGTCAATAAGA
CCTAGGCTTGTGACTTTAGCCATGCTAGGTATTCTGGCAGGCTAACC
GCTTCAATACAAATAGGCATGAAGGCGTGGTAAGCTCCACAAAGTTC
GCTACATTGTCCGTAGTAAACACCTTGTCGTTGGATCAGAGTAGATAC
TTGGTTTAGTCGACCTGGACAAGCATCGACCTTGATACCTAGTGAAG
GAATAGCCCAGTCATGCAGAACGTCAGTAGCTGTTACGATGGCTCGG
ATGTGCGTGTCAACTGGAAGAACTAGCCGGTTGTCCACTTCTAGTAG
TCGTAGTTGCCCAGCTTCTAGTTCGTCAGCAGGAACCATGTATGAAT
CAAATGCAACTGTTTCGTTGTCGTCACCAGCAAAGTCTGATAGTTCGT
ATGACCAGTACCATTGGTGTCCAATAACCTTAACAGTCATAGCTGGGT
TAACCACCTCCATGTGGTTCCCCTTGTAAACCTATAGAAAAGGAATAG
GGTTGATTATGGACTATGCCTTGCTTCCCACCACGATAGTTCATAACT
ATGTTAACAATCCTGAAAGGACTTACCCCGACCCTCCTAACACCTAGT
CTTTTTTATTACCCTACCCCCCCAAGGGGGGGTTAAAAGGTGGGTTA
GGGCGTAAGGAGGGAAGTAGGGGGTTAATGTGGGATTATAGCACCG
TTGTAGTCTCTGAGGCCTACGGCCTACCTGTACGGGATATAACCCAG
CCAGGTATACAGTGGTAAATGCATGGACGAGAGTAACTGTGTTGATT
TTTCCAATTTTTTTACCTATACTGAGCCTAGTATAGGGGAGGTTTAATT
AAAATTATCAAACACAGCTCATAGCTATGATTTCTATGTCCTATAGGG
AGTCCATGAGGTATAGCAGCTTGAATGAAGGGAAGGCAATAGCCACA
AGGATAAGTGCTGGCACAACAGTCCATACAACTTCAATAAGAGTACC
GTGGTTGTTGTACCGGTGAGCGATAGGCGCTTGTGTGTCCTTGAAAG
AGATAACGATAGATCCGAGCATCCATCCTACTGCCACACCAATAACG
ATGAGGTAGAAGAAGATAGTGTCGTGTAGTTCAGTGATCCCCTCGAA
TGATGGAGAAGCACCGTCTTGGAATCCTAGAAGCCAAGGTTCTGGAG
CGTCACAAGTAGCTGTGAATAGTGATGTAAATAGTGAAAGCATGCTAT
ATAATGAATATTAAATATAAACTAGTCGTGTCAATCGCTACCAATGATA
AAACACTGATAGTGGTACTAGACCAATGTGACGTCATAGTCACTTTAT
TAATCGCATAATACAACCTTGTTGAGAGGTGAAAATGAGATAATAGGT
NNNNNNNNNNGCGGACGTGTCAGGTAGTAAGGGGTACTGATAACCA
TAAATGGTTACTAGCTAATAACGGTTAGCTGAATCGAACAGCCTTTAC
TAATGAGATCAACCCATTCTTTAGTAATCCAATACCGTGTATATTAATA
AAAGTCAGTCTAAATAAAAGATATTGTATTGTTAATAGACAACTCTATT
ATAACTATTTGGTAGGTAAGAAGAGAAATATATTACACACCATAGCTA
TAGGTCTCTCTAAGTACAATGAGAGTGGTAAATAACCTATATCTATTA
GGGGAATATTAGTCCTTATGACCTAACAGCCCCAACCCAATAACTAAT
GGGTATGTAAGAAGGTTAATAAGAGGTTGGTAGAATTTCTCTATTGCT
ACTAGTGTATAGGCCTATACTTATAACTATAGGTCTCTACTTATATATA
TATATCAGTTAGGTGTACTTATAACCTATTACTAGTAGTGTATTATTAA
TACCAAAAGGTGAGTAGGGGGCTAATTTACCATTCATCTATCAGCCG
CAGGTTCCCCTACGGCTACCATGGTTCGACTTCAGACCAGTCAGCTG
GCCAGGCTCTGTAGCAAGCTCCGCCATTATTACTGTCTTCCAGCACC
CATTGATATCAATGGGAGGTCTCTCCATCATTATGACTATAGGTTGAT
GTATAATTATAATGTGGATATATATGAGACTATAGTTAAATACAAGCAG
CGCTCACCCACGTTACCCTACCAGCATACTAGCCCGTGACGAGCGGT
TAGTACCCCAGAGAGATTAAACTTCACCGCTCCATACTTATGAGCGAT
TACTCGGGACTCCAGCTTCACGCGAACGAGTATCAGCTCGCGATCTG
TATGCTTGTGGTAGTTTTGGATGCTTTCCCATTTTATAAGGCTAGCTC
TCATCTGACACACACACTTGTGGCACGCCTGTAGCCCTTCCCATAAG
GGCCATGACGGCCTGACATAGGCCCGTACTCGTATCCCTTATAAGGC
TACTACTGCCTATACACTAATCAAATTAATTAGCATCATAGACAGACG
GGTCTGCGTTCGTTCATTGGACTCAACCGTAGGGCTCACGACCACGA
ACTGACGACGGCCATGCAACGCCTGTAAATTACCAGACTCGCTCTAA
ATTAATAAACTATTAAATAATGAGCGAGCCAACAGTAAAATATGCAAG
GGAAGGTAAGGTTTTAAGCGTATCGTCTGATTAAACAGCATGCCCCG
CGCCCGTGTGCTCTCCGGCGGTCTCATCGTTAGTGTGTCGGTCTTGC
GACTATACCCTACAGGCGAGTTGCTCAGGCGTTAGCCTTCCAGGGGT
TAAAATTGTGACTATGCTTGACGTCAAGTATTACACTCTATATTCAAGT
CGCACTCCCCACTGCGGCAACTATGGTTCACTGCTGGGGCTAAACAG
GCTCTACTCTGTGTCGGTATCCCAGCCTTCGTACCTTACCGTCAGTCT
GTCACCAGCACCAATTGCATTGCCCACCCCTAGGGATCATAGGATCT
CAGCCCTCCCCCCAGTGTTGGTGGTACCAAATGTAGACTCGTATCCT
ATTAGAGATGGCTGCGTACCCTATACGCCCATTCAACACCATAAGAG
CTGACCCGTCTGTGTTACCGCGACTGCTGGCACAGATGGTTGGACG
GGTCTTGTACATAATAAGCATCAATATGACTTATTATATCTGGATATTA
AGATATCCAATCTAGGTCACCTGGTCACACCTTTTCCCTAACCCCCCT
ACGGGGGGTTAGGGACGCGGTGCATTGCCAAGGATTCTTCACTGCT
GCGCACATGGGTGCTCTGGCTGGCCAGTGTCGGATGCATAGCTATC
ACTATCCCCTTCCCGTCATAGCCTAAGTAACCTCATAGCTGGTAGGG
GCTGAATCTACGACAATATGGTGGATTACCTTTGCACCATGTATAAGC
ATTAACTGATATCACACAAACTCTTAAAAAAATAAAAGTAGTGATCAAT
TTAGAATACTATTCATGATGATTATCCCTGCGCTATAGGGGCCATTGC
GTATGATTCCAGCACTGCTGACCATTTGGCTCCTATTCACCATAATGA
ATCGGAAAACTTGCATGTGTTATACGCCTAGATACCCTTCATGGCTCG
CCACGATGAAACGATCGCGATTATCCCTTCTATCAATACCTATATTATT
ACTTATTATCATCAATATAGGTACTACGCCCCACTAACCCCTTACTAA
CCCTTACCCCTCCAATGGAGAGGTTAAAAGGTGGGTAAGGGCTTGG
GCGGAAAGGGTCTAGATCATCCCAGTTACCACATTCAATNNNNNNNN
NNATTGTGACTATGCTTGACGTCAAGTATTACACTCTATATTCAAGTC
GCACTCCCCACTGCGGCAACTATGGTTCACTGCTGGGGCTAAACAG
GCTCTACTCTGTGTCGGTATCCCAGCCTTCGTACCTTACCGTCAGTCT
GTCACCAGCACCAATTGCATTGCCCACCCCTAGGGATCATAGGATCT
CAGCCCTCCCCCCAGTGTTGGTGGTACCAAATGTAGACTCGTATCCT
ATTAGAGATGGCTGCGTACCCTATACGCCCATTCAACACCATAAGAG
CTGACCCGTCTGTGTTACCGCGACTGCTGGCACAGATGGTTGGACG
GGTCTTGTACATAATAAGCATCAATATGACTTATTATATCTGGATATTA
AGATATCCAATCTAGGTCACCTGGTCACACCTTTTCCCTAACCCCCCT
ACGGGGGGTTAGGGACGCGGTGCATTGCCAAGGATTCTTCACTGCT
GCGCACATGGGTGCTCTGGCTGGCCAGTGTCGGATGCATAGCTATC
ACTATCCCCTTCCCGTCATAGCCTAAGTAACCTCATAGCTGGTAGGG
GCTGAATCTACGACAATATGGTGGATTACCTTTGCACCATGTATAAGC
ATTAACTGATATCACACAAACTCTTAAAAAATAAAAGTAGTGATCAATT
TAGAATACTATTCATGATGATTATCCCTGCGCTATAGGGGCCATTGCG
TATGATTCCAGCACTGCTGACCATTTGGCTCCTATTCACCATAATGAA
TCGGAAAACTTGCATGTGTTATACGCCTAGATACCCTTCATGGCTCGC
CACGATGAAACGATCGCGATTATCCCTTCTATCAATACCTATATTATTA
CTTATTATCATCAATATAGGTACTACGCCCCACTAACCCCTTACTAAC
CCTTACCCCTCCAATGGAGAGGTTAAAAGGTGGGTAAGGGCTTGGG
CGGAAAGGGTCTAGATCATCCCANNNNNNNNNNATCATCACAGTTAC
CACCTTCAATAAACTTATTGGTAGGTTGAAGGCTATAGTCTGGATCTA
ATGATCGTCTTATGCGTAATTAAACGCGGTTCGTGTTGTACTATTAGG
TAAAAATTTTGTAGCTAAACGCTCTAAGTTGATATAGCTATGATAAGAG
CGATGAATTTATTGACCAACTTCTGAGTCGTCAATGGGGACGAATACT
TTACCTAATCTGTATGATACACCATTCATAGGCATAGGGTAAATTAGA
ATCTATATTACTAGCAAAATTACCCCTATCAAAGTATAATCTTTCGTAC
TAGGTAATAACATGGTGGTACTCTTCACTAAGTTTTACCATTTATCAAC
TCTAGAAGAGGGTGTAGTAGGAGGGGTGCGAAACCATAAGGACCAC
AGTGGACATTATTGATAGTTGGTGGGAATAATTAGTCATCATCATCAT
CATAACAATAGGCCATGATAAACAGGGTATTTTTTGTAATCAATAATTA
TAAATATAACTATTACCAGTAGTGATGAGTTTATAATTATGCCCTATAA
CTGTCTATGTTTTCCATTCCTATGGTGATTATAAGGGGTCTATGCTAT
GATGATAGGTTGTGTAATGCTTATCGTGATAGCTTTGATGGAGCGTTG
TCACCAGGTAGTAGTGAAACAGGTGTAATCGCTGAACGTTGGTCTGT
GATGGCCAGAACTCCAGAGGCTACTTCTACTACGAATCCTAGTGTCA
GTACTAGGAAGAAGATAACGGCAACCCAGAAACCGTATAGACCAACG
TGTCCAAGGCTAGGAGCAAGAGGGATCAGTGTAGCCACTTCTAGGTC
AAAGGCTAGGAATAGCACAGCAACAAGGTAGAAGCTGATACTGAATG
GAGCTCGTGTTTGTCCGCGAGAGGCGTCATAGCCACATTCGTAAGTA
GTGACCTTTTCAGTGTCAGGACGAGCTACAGAAAGTAGTGCGTTTAC
AAGCAGTAGAATCACGGCCAGTACAGGCACAATGATAACAAGGCTGT
ATAGAGTAGTACTCATAGTCATGGTTATACGCTAAATAGTGAAAGAGC
CAGGAGACGAGCGCTATCTAGTAGTAGTGATGGTGATGCCATGTAGA
AGACTAGGATGGCAGTAAGTAGAGAAATAGTGTATGATTGTACAGCT
GTAATGTCTACAGGCTTTTCTGAGTTACGCATAACAGCCCAGTAATAG
TCCCATCGAGAGTTAGCAAAGTGGTCATGAGGGAATGTTTGTGGGCT
GACTGGTGCTGATGGGTCAAACCAGGCTACTCGAATAACGTTAAGGT
AGTATGATGCACTGATAACGCTAGAGAGGATAGCAACTAGTGATACG
AAGTAGTACCCACCACTGATAGCGCTGTAAAGCACTTCTAGCTTACC
GAAGAACCCTAGTAGTGGTGGAACACCAGCTAGTGAGAATAGTAGTA
CTAGTAGGCTAAAGGCTAGAGGTAGGTTTCGTACACGTAGACCAGCT
AGTTGTGAGATCAGTGTTACGTCATGGGTAACGTCACCTCCACGAGA
TGAGCTATGGCTAATAGCTCCTAGCAGGCTGAATGATAGGATAGCTG
TGAGTGTGTATTGTACTAGGTAGAACACAAAAGCGCTAGTAGCTTCA
GGGCTGTTTACACTCATAGCTAGTAGCATGAACCCTACGTGTGAGAC
TGTAGAGTATGCTAGTAGTCGCTTGATACGTCGTTGTGCTAGACCAA
CTACAGCACCTACAACAAGAGACAGTGTGCTACTCATAAGTAGTAGTA
GTGCCCATACGTCGTAAGAGGCTGTATCAACAACAACAAGCATAGCA
CTGATAAGACCACTGCTTAGGGTGTATAGTAGTGTCAGTAGGCTGAT
CTTAGGTAGAACTGCAATCCATGTAGTCACAATTGTTGGTACCCCGTC
ATAAACGTCAGGACCCCAGTTGTGGAAAGGAGCAGCTGTAACCTTAA
ATAGAAGACCAGTCATAACAGCTGTAAAGCCAAGAGCAAGAGCCCAG
TTGATGCTGATAGAGTCTGATCCACCAACACTCAGAAGTGTGACTAG
AGATTCAAGGTTAGTAATACCTGTTTGGTTGTAAACTACGGCAAAACC
TAGTAGGATAAGACATGATGATAGACCACCTAGTAGGAAGTATAGTA
GACCACTATGAGTAGCAGACTCTGAATCACGGTAAAGAGCTGCTAGT
ACATATACAGCAAATGACTGTAGTTCTAGAGCCAGGTATAGAGTAACC
ATGTCACCGCTAGCGACAAGGAGACATCCACCCATGCTAGAAAGAAG
TGCAAATAGAGGATAAGTGGCAATACGAGGAGTAGCCATAGGTGATA
GATGAGCGTGAGGAGCCCATTGGACTAGAGCAATAGCACCTACAGAA
AGTAGGAATACTTGTGCAGCTAGTGAAAGACCAGTCAGATCAATAAG
TCCACTGTATAGACCAAGACCTGATCCTACCCCCTCCCAGCTATAGG
CATTGGCGGCCAGTGCAGCAGCTGAAAGGAATGTGAGGACTGTAAC
TCGTGTGAGTAGAGCTGGCGTAGCATAGACCGAAGGAAGCGCAACG
GCTGTCAGTAGAATAAATAGAGCTAGTGTAAGCATTACTGTTGGCGG
TAATAATAATAGTTGTTATCTCAATAATCATAAAGAGCTATAGTACTAT
ACCTACTCCCTATTATAGACCCTATTCATGCCTAATAACTATAATATCC
AAGAAATCTTTGATTACTAAATAACAACTCTATCATTATATATGCCTAT
ATTTTCTGGGTATATTAATTAGACGACAATCCATAGATATGAATAATAC
ATATGGCTTATACCTACCTATAGGCTTTAAGAATTAGATCCTAATACCA
CCCCTCAGTGTTAGGCATGCCATGTCTTTAATAACATTACTATTCTCTA
TCCCTGTGAATCTCGTAATACAATAAAAAGTATGTTAAGAGTATGTTTG
TTTTAAATTTAAATCTACTCACTAACCCTATGGCCATAGGACTCAAGCT
GTACCGTTAATTAGCATTTAACTATAATGATACAAATTAGAAATCATAT
CCTAAGGAATATCTGTCGTATAGGTTTTAATACAAGTACCGTGATTTG
CACACGGACCATCTGCTTGGAAGGCAGAGACGCTACTCTTACGCCAT
ACTTGTCAGGGAAAGGGTCATTTAATCTATTGTGATAAGTATATTTAC
CATTATCGGTTGGGCTACTTCACTGTACTATTTAAATGGTGAGTCTTG
TACTATTAGAAGAATAAATCAGATATTCTTAGTAACACTTCGCTTGAAT
AGGATTAGGTATAGTAAACAGTGGTACTACTTCTTGTTATACCCTCTA
CCACCCCTACTAACCCACCAAAGGTGTGTTAGTAGGCAGGTTAAGTG
GGTAGAGGGAAGGAAGAGAAGGGTTAGTATCGGAGGGATAATCTTAA
TGATTGTAGACGTATTATTACTTATACCTCATAGGTGGTGTATAGAGTT
TATAATTTTGATCCTTTAAAGAGGCTATATAAATGGAGTGACACGGAC
TCGAACCGTGCTCTCAGCGATGCAAACGCAGTATACTACCAACTGTA
CTATCACCCCTCCCNNNNNNNNNNCACCCCTCCCTTAGCACTAGTTA
TTTAAGTTTTTTCTATTCTAAGGGGTCTATAGATAATATCTATTGTCGT
GCTCGTAGACATAGAGCAATAGGACCCAGCATCGCTAGTAGCAGTAC
GAAACTGATAAGAACGAGCCAGAGGAATCCATAGGTGTAAAGGCTAA
GACCAAGAGCTTGCACTTGTGATAGGTATGCGTATGAGGCATCAGCT
GCTTGTGAACCGTGTACAACGAATACATCAGTCATTGATGATGTAATA
ACTGGTGCTCCATCACCAAGAAGCAGAGTGTTAAGGCTGTATAGTGC
TCCAGTGATAGCGCTGACGGTTGTTGATGTTACGCTAGGCATTACGC
TAAAGGCTTCCAGTAGGAATACTCCAGCAATGATGAAAACTAGTGGTA
GTCCTTGAGTAAATTCTTGCCCGCTAGCAGTAATTTCAGAGAGACGTA
CGTTAAGCATCATCACCACGAATAGGAATAGCACAGCAACAGCTCCC
ACGTATACGACTAGGTATGTGAGACCTACGTACGTGATACCTAGACA
TACTAGGTAACAAGCAGCTAGGACGAATACACCAATTAGATATAGGAT
AGCTACAATAGGGCTGCTAGCTGTGATCGTCATTACAGCGCTTACAA
CAGTGGCTAGTGCCAGTAGTTCTAGTACAAAGCTAGTCANNNNNNNN
NNGTGACAGTCATGCTGATACTAAGCGATAAAGTAAAAAGCATTTATT
CACTGTCATAGCATCCAGGTCGAGCTTGCTGCCATAGATGTCCCACT
GGGAATACACATCGTCACCACTGTGGATCGAACACAGGTCGGAGCAT
TAACAGTGCCCTGCTCTACCACTGAGCTATGGTAACCATCGTCTATCA
CCCCTCTTATAGACCTAAACTATATGCTTGCTTATTAAAATGGTAAAAT
ACTATCACTAGTTACACACTTAACCATTTATTAACCCCTCTACCCCTTT
AGTTACACTCTTATACCCTCTCCCCTGCCTATCCTCTTACCCCCCCCC
TTGGGGGGTTAAAAGGTGGGTTAAAAGGGGATAGGGGGTCAAGATG
TGATCCCATTAATTTTCAATTTATTTAAATTAATTTAAACTAGTGAAGTT
CAATAGCGTCCTTCAGGTATGAACATGTTAGAACACAGAATACGTATG
CTTGAATCAGAGATACAGCTAGTTCTAGACCAATAAGAGCTACAAACA
CAGCAAATGGAATTAGTGTGATAACTGCTACAATAACTCCTGCGCTGA
ATAGCTTAGCTAGGAAAGTAGCAAGAATCTTCAGTAGTGTGTGACCA
GCAGTCATGTTACTGAATAGCCGAACACCTAGGCTAGCAGCTCGTGC
AAGGTATGAAACTGTTTCGATCAGTACCAGCATAGGAACCAGAGCTA
GAGGAGTACCGCTTGGTACGAAGTATGAGAAGAAGTGTACTCCGTGT
AGACGTAGTCCTAGGATAGTTACAGCAAGGAATACAAATACTGATAGT
CCAAGAGCAGCAATACCGCTAGTAGTTACTGTGTATCCGTATGGAAT
GTTACCGTTTAGGTTAGCTACAAGAATAAACATGAATAGCGCGTAGAT
GAACGGTGTGTAGATTTCGTGACGGCTACCTACTTGGTCTCGAACCA
TACCTGCAAGTGTTGCGTAGGCAGCTTCTAGGGCTAGACTCCATCGA
CTTGGTACTAGGCTGTACCCGTTGTTAGCTACAACGTGAAGTGATACT
AGAAGTCCTACAGTAAGGAGTGTGTAAAGACCTAGGTTAGTAAGAGC
TAGAACAAAATCACCAACCACAGGTACGTGGAGGCTAATGAGAGGAA
CAACTTCAAATTGTTCAAGAGGAGAAAGGATTACTGCTTGTGTTGTCA
TTTATTGAGTTTTAAGATTAATTAACAGATGTTAAGAGGTGTTGACAAC
AATTATTAGCAGTTTTTAATAGGCATAATAATATATTAAACCCTAAAAG
AATTTAATAGTTCGAAAAAGGCTTAATAAGCAAGAAAAAGGAGGCAGG
TTTAAGACCCCTAATAGGTTTGGTTTAATAGTTGTTAGAAAAGAGCTTA
ATAAGTAAACTGTTTAAAACATTAAAAGGTTGTTATTACTACTCTTACT
GAAGATTTATTAATCTAGGCTAGGGAGCTTCCTAGCCTCTTATTTTCA
AACAAGGAAACAAGAAAGGNNNNNNNNNNATAGTTTGTAATGATGAG
CTTGTGAGCGGCCTCACGCGTGGACTGCAACTCTGTGCGAAGGGGT
TCAACTCCCCTCAAGCTCTATTCCTTGTCTTGGCCTAGCCCATAACTA
TTATGATAGTTATGTCTCATAGCTATTTAATGAAAAACCATTCAAAGCT
ATTGATTATAGCCTACTTATAGTTATTAATAATAAATCCCCTCTAGACC
CTTTTAGCAATAGGGATGGTAATAGTGGGTTGTAGGCGTACAGTGCT
GAACGGTATCGCACGCCTTTTTATTTTTTATTAAAAAAAAAATCAGACC
GTAGGAAGCTCAGAGAGCCATATATAGTATGCCTTGCCTTATAAGCTA
CTCACTATACAAATCCTTCACTAACATTATCATTCCCTTACTAACCCAT
ATATATATATATATATATTTTAATTGAGAATGAGGGGAAAGTGTTAGTA
ACATGTGAGGCAGTTATTACATGATAAGGATAGCACAAGCTATGGCA
ATAGGCATAGAAACCTATGGTTATTACTATCTGGGATTTACCCATAAC
TAGTGATTATCCATGAGAAGAGCCACCCATACTAGGCCCATGTATATA
GACCATATCAGCATCAACAGGACTCTGGCTATGAATGTCTATAGCCC
CCTCCTCCCCACCATTTCTAACGCTACAACTTAATTTGTGTACTCTCC
TAAGCATAAAGATAATCTTATTAGCATAAAAAGTAACCAACCTAATAGT
ATAGGTCTAGTATAGATATTTGTATTTATCACTATAAGTATGGTTAATG
TATCACTTTTATTAAAATATTAAATAGGGGTCTATTTATCCACTAATAGT
CGGTGTGTAAGCATAACCTTGACTGTATACACGTTCTTCTTGTTGATG
GCTGTGGTAGAGGCTGTGTCTAGCACTTGTTGGTCGTTAGCCGTTAG
TGAACCTACAACGATTCGTTGTTCTGTCATACGTGGTCGTGCTGGTTC
GGCAGATAGACGACCATGAACTGATACTTGTACCCCTACCAGACTAG
CTGGTAGTTGTACTCCATTGACACTAGGGTAGGTAGTGATAGGGGTA
GCATTAGATACTGTACCAATAGTACCAAGCATACGGTTGATAACCCGC
CCAAACTTCTTAGTCTTAAGTTCCCGTGCTAGGTAGTGACCAAGAACG
GTAGCGTCCATATATGGGCCTGATAGTCGTGTAACGATTAGCATTACT
GGTCGTTGGCATACTTCGGACAGTGCCGTTCGTAGTTGCATAATAGT
TAGTGACGATAGGGCATGGTTTGATGGTGCGTAGTAACCGACCTTCA
CAGTAATACCTCCTACAGGAAGTCCAGCTGCAGGACGTGTAATGATT
GTAGGTCGTCCCCAGGCGACTTGATGTCCAGCCTCAAAATGGTTGAA
TACCTTTAGGGTATTGTTGATGGTGTGTAGTACACGTTCGTCTACGTA
AGAGTCTGATACGTATGTGTGTAGTGCACCTGGTGCTAGTTGTAGAT
GTGCCATATTAATAATGATGAAAAAAGTGTGCGACCAATGGATAATGA
TCATAAGGATGTATTGAGATGGAACAGTTAATTGCTATCGAGCATATA
TTATCATTCATACTGATTCCCGAGAGGAAGGATTGAACTTCCGTATTA
TCGTTATGAGTGATACGCTCTAACCAACTGAGCTATCTCGAGTATTAC
TCTCTGTTACTAATTTTATCTATATAGTTAGGAACTAATAATCGTAAAC
TTTTTTAGGTAGGTATCTAAACCATGCACTGATAGGTTCTATAGGACG
TATTGGTCATAACTATGATCATTTATAAGCAGTACGAGATTCGAACTC
ATGACACACTGGTTAAGAGCCAGGCGCTCTACCGCTGAGCTAACCGC
TTAACACCGTACTTTTATTAGGATAGGGTCCTATAGGAATGTTATCCA
TCTCATGCTGACCAACTACGAGTCGAACGTAGACAGACTCCACCAAA
AAAGAGCAGGGCTACCATTACCCCATTGGTCATACACTGTGCACCAT
TACCTACCCTCCTCTACCCATCTTTTCTACCCTTTACTCCCCCCCCTTT
TTAAGGGGGTTAGTAANNNNNNNNNNGACAGAGGTAACAAGATGAAA
TACTGCTTATAGTGATGGACAGCTGACAGGAGCACGACTCGAACGTG
CAGTGGACAATAGCCCGACTGGTTTACAGCCAGCTCCCTTACCAATT
AGGGGTACCCTGCCTTTGTTTATACGATACTGCCCCAACGTGGAGTC
GAACCACGGCCTGCTGAACTTCACTCAGCTGCTCTGACCAACTAAGC
CATCGGGGCTATCAATGCTCTATTAGAGATTGCAGATATCTATAACGA
CAGGTGAAAAGCAAATGCTGATAGTTAATAGGCAATGAACTTGCTAG
GCACAGAATGATAGAAAAATATTGTTACAAAAATGGTAAGAGATAATA
ATACATTGTAAGGAGGCATAGTACAATCCTACTCTGTGCCTAAACACC
TAGAACGATTCGAACGTCCATCTGTCGGGCCGAAACCGAAGGCTCTA
CCATTGAGCTATAGGTGCAGGTTCCTAGCTATCGACTATGAAAACTTT
CTCGATTAGGGGCATTGACTAGCACTATCATCAAGCATAGGAATATAT
AGCCTGATGGGGTAATTACTGTGGTAATACATATTTTCTATAGTATTTA
GGTAAACACGCAGCCTTATATAGTATTTATACGTTTGATAGGACTCGA
ACCTACACANNNNNNNNNNGCTACACATCTCTCGATACACGCTCCTA
AGGCGCGCCTGGCTACCAATTACAGCACAAACGTCATAGTGTTCTAT
ACACTATTAGGGCATAGAATACCTAGAATAAAATAGTAGATGCATTGC
TTATTACCACAACTATGTTACTATTCATAATACTATCACCATGCCTATT
AATTATGGAACCACTATGATGAGTTCTATTATCTATACTCTAATGAATG
AAAGGGGTCTATAGTATATCTGTTAGCTATGATTGTAGTGATGGGGCT
GTAGCAGCTGGAACATCAGCAGCTGGCACGTCCAGGTCAGATAGTG
TGTTTTCCACAATACCAACTACAGGTACAATAACAAGGTACCATCCGA
AGTAGAAGATACTTGCAATAGCACCGATAGTAATGAACGGTTCTTCAG
CGTGTTGGCTACCAAGCCATCCTAGTAGTACTAGGTCAACAGCTAGG
AACCAGAACGCTACTCGCCATAGTGGTCGGAATTGTCCACCACGTAC
ACGGCTAGTGTCCAGAATAGGCATAGCTAGCAGGATAAGTAGTGATC
CAAGCATGGCTACCACACCTAGCAGCTTGTTAGGTACGGCCCGTAGG
ATAGCGTAGTAAGGAAGTAGGTACCACTCAGGCACGATTGAGGCCG
GTGTACTCATAGGGTTGGCTGGGATGTAGTTGTCGCTGTGACCTAGC
AGGTTAGGTGCGTAGCACACAATACCTGTGATGGCAGCAAGGAATAC
CCACATAGTCACCAGGTCCTTGAATAGATAGTATGGAGCCATTGGCA
GCTTATCGCTGTTAGATGAGAGTCCAAGAGGGTTGCTTGACCCGTGT
GTGTGCAGCGTCATCATGTGCACTAGAGCTAGTGCAGATAGAACGAA
TGGTAGCACAAAGTGAAGAGAGAAGAATCGGTTAAGCGTAGCGTTGT
TTACACTAAATCCACCCCAAACAAATTGTACGAAGTCTTGTCCGATCC
ATGGAATAGCTGAAAGCATGTTTGTAATCACAGTAGCTCCCCATAGTG
ACATTTGACCGTATGGTAGAACGTATCCAAGGAAAGCAATAGCCATC
ATAAGGACTAGGATAATTACACCTACTGACCATGGCATTACCCGTGGT
GCCTTGTATGAAGAGTAGTAAAGACCACGTCCGATGTGGGCGTATAC
TAGGATGAAGAAGAATGAGGCACCGTTAGCGTGTAGGTACCGTAGTA
GCCAACCGTAGTTAACGTCACGCATGATATGCTCAACACTCACAAAA
GCCAGGTCTACGTTAGGTGTGTAGTGCATCGCTAGAATGACACCAGT
CACAATTTGTAGACCTAGACATGTGGCTAGTAGTGATCCAAAGTTCCA
CATGTATGATAGGTTCGCAGGTTGTGGACTGTCGACGACGTAAGAGT
TTACTAGTCGTAGTAGTGGGTACTGCTTTAGCAGACGCATAGTACACT
AGTAATGTCCACCACATGGATTACAATGAAGCATGATGGACAGGTAT
CCCCGCAGGGACATTAAGAAGAGAGATGTCATGTTAATAGTGATAAA
TCTAACATGTTGTTAATGATAGTGAGGTTATTTAGCAGACTATTTCAGT
ATGATACGTTACCAACGCTAGACGTATGAGGAGTCGAACCTCAATCA
CCACTATGAGTGGTGGACTACCAGCCTAATCTGATACACATCTTGTAC
AGTTTTAGACGTAATTAGATATCCATCACCCTTTTAACTTTCGTATACC
TCCAGAGACTCATGTATAATATAAGAACAGTGATACGACTCTATAGTC
ATAAGTACAGGAATCCCACCCCTTGCTATAAAAAGATATAATATTACC
ACATTCACACAGACGTTATCAGTTTTATCATGTTTAAGAATATCCTTTA
CCCGGATCATACTTAAGAGCATTATAATTTAGATGTGTTGAGGGGCTA
GGTCCACGTAGGGGGAGTGTAGTGCATCGCCAGAATCACTCCTGTAA
CGATTTGTAGACCTAGGCATGTAGTAGTGACCCAAAGTTCCACATGTA
TGATAAGTTCGCAGGTTGTGGACTGTCGACGACGTAAGAGTTTACCA
GTCGTAGTAGTGGGTACTGCTTTAGCAGACGCATCGTTCCTACTTAC
CCCCCCTGTAAGGGGGGGTAAGGTGGCCATAGAATGGATTACATTAA
TGTCCATGACCGGGTACCCTATAGGGGCCTTACCTTAAAAAATAAACT
TCCAGAATAGGCATGGTGCTCTGATTAGAGGGTGAGAAAAGAAAAAT
ATAATTTCAATAAATAGGGCTATTACAAGCTTTAAAGCCTTAACGTTGC
CATCGCTAATAGACATACGAGGAGTCGAACCTCGCCCGCCGCTACAT
CAAAGCGGTGCACTAACCAGATATGCTATACGTCTTGTCTTGTTCTGT
TATGCTTAGTTAGTTAATTATAACGTTTCAGTATTCAATGTTATTAATG
CCTGTAATTTATTACGGTTAACTATGCTTATGAGCAGGAATATAATGAA
TATATTTATTAAAAGGCTGGCTACCTTATCCATATTAGAGCAAGTTGG
CTACTTTTATGGATATTTAGAAAAAGGGTCTATAGTGATGGTACAACA
TATGCTAATACACTATGACTTGCTACCTTGGCTTACTGATGGTAGTAG
CCCTAGCCCTGCTACGATTAGTAGCACTAGAGACACGTCAGTAACAC
TGGCCGATGGTACCCCAGGTAGTACTAGAGGAGCGATCACCATAAG
GGCAAGTGATAGGAAGCCTAGAACAATGTAAAGGGCGTAGTTCGTTA
CGACTGATGTGTCGTAAGAAGCAATAGTACGTCCCATGTTAGGCAGT
ACTGTTGATAGTCCATAAGGACCAACAGTTTCGATCAGACCACGGTC
AAGAACCTTAGATGCAACGTATCCCAGGTTCATGAGAGGCGTGATTA
CTAGACCTGTTACAGCAGCGTTCCATCGCCATTGTCCGTTGATGAAC
CCGTATAGGGCTCGTGCTAGGGCACTGTCGGTTAGGCTTACAGCCA
GACGCATACCAGGACCGTATAGGAATACAGCTCCCATTGCTCCGAGT
AGTGTCATAACCAGTGGCAGTTGCTTGTATAGTACTGGTAGGGCAAA
TTCAGCTTCTACCAGTGATACGTGAGAAGGGTGTTGGGGCAGTACCG
CGCTGAGGTAGTCAGTACCCATTCCTAGCCATAGGTCCTTAGCCATG
TATCCAAACAGTACTGAAAGCACTGCTAGGAATACAAGTGGAGCACC
TAGTAGTACTGGAGCTTCGTGCACGTTGTCGTAAGCTTGTCGTGTTC
CATTGACAGTACCAAAGAACGTTAGGGCGATCAGACGGATGCTATAG
AACGCTGTAAGACCAGCAGCCAGTGTACCGGCCCAGTAGATTACTGT
TCCGTTAAGGCTGTATGTTCCCATAGCTAGTTCTAGTAGATGGTCCTT
ACTGTAGTACCCTGTCAGTCCTGGCAGGGCCATAAGTGACAGTGAAC
CAATCAGCATAGCCGCGTATGTGAACGGTAGTAGTGGAGCTAGTCCA
CCTAGTCGTCGTAGGTCTTGTTGGTCAGCCATAGCGTGGATAACACT
ACCAGCAGCCATAAACAGTAGAGCCTTGAAACAGGCGTGTGTAACAA
GGTGGAAAAGCCCTACGCTGAATTGTGATAGCCCTACAGCCATCATC
ATGTATCCCAGCTGTGAACATGTAGAGAAAGCGATCACTCGCTTTAG
GTCGTTCACTAGTAGACCACATGTAGCAGCGTAAGTAGCTGTTGCCG
CACCGAGCCAAGCAGTTACTACAAGGGCGGTAGGACCGTACTCTAGT
AGTGGTGATGTACGAAGCATAAGGTATACCCCAGCAGTCACCAGCGT
AGCAGCGTGAATTAGAGCAGACACAGGAGTTGGACCCTCCATGGCC
AATAATTGTTAGTAGCAATGACAATCCCTTTCAGGATTGAGCAGGCCA
TGTCAACCATCAGGAAGTACTTATATGCAAAAAAATATCTTGAATCAT
GTTTAAGCATAACAACCTATGGGTACTAGTATGGTCTTTGGGCATGAA
TCACAACTAGTGATTCTATGCTGCCAGTATTGAATAGTTACAAATACT
GGCAAATCATAGTACAGCAGACGCTTTAAAACCAAGACAGAAAAAGG
GGACTATCGTACAAGATGCCGAGCCCATACGGGTAGTTGAGCACCAA
TAAGCTCGTCTAGTACATGAAGGTGTTCAGTAGGTACCCAAACGTAG
TAGAGAGTGGTACGTCGACCATGTGGGGCTGGGCGTTGGGAAGACA
GGGCTGGTACACCTAGGGATCGTAGTCGTGCAGCGAATAGGTCGTA
TTCGTAATCTTCAGCACCACTCCAAAGGGGTAGTACCAGTCCGCCTG
CTGGTTGTGAGAAGTACTCACGCCAAGTGTCTCGATAAGCGCTCAGA
ATAATATGAACAACAAGAGAATGAAGATTGTAGCAACCCAGTGCTTGG
GCTGTTAGACGTCGTACACCAGGGCCTGCAGGCGTCAGGTAGAAGA
GTGAATACAGAGGCCATAGTGCTGGTAGTGTACTTGTGAGCATCAGA
GTGAAGCTGACCGGTGGGAGGTGAGGACGACGGTTATCTGTCACCT
CAGTAGTAACAGGCTTATTGACCATAAACATGGTCATTAGTGATTGAA
CATATTCCCACTCAGTTTGGCTCCACATAGAAGAAATACGCTTGATTG
ATAGCCGTGCTGTGGGTAGACCACTAGTCAGGTACTTGCTGAACATT
TGGAATGGCCCCGCCACGAGCATAGCTACGAAAGCTGCAAGTTGATC
AGGGCTCATAGAGGTGGGCATATCCTTCTTAGCACTAGCACCCTTGT
TACCATACGAACCAGGATTAGTACCAGGACCTGTCTTTTCACCTGAAC
TTGATGGAGTAGTACCCGTACCCACCTTTGTGGGATCTGTCCCGGTA
ATAAGATTTAGTTCATCAGCTAGCCACGTATGTAGTGGTAGTTGAGCA
CTCTTACTCATAGCCCCACCTAGGAAGAGTAGACCAATAACAGTTAGT
ACTGTTTCGTTTAGAAGAGGTGCTGTGCTCAGAATAGTTGCGTAGTC
CAGGCTACCAAAGACAGCGAGAGCCAGGAAGAAACCAATGCTGAGT
GAGGTGTCACCAACACGGTTAACCATCATGGCCTTGTTAGCGGCCTT
AGACGCTTGTACCCGTGTGAGCCAGAACCCAATAAGGAGGAACGAT
GCCACCCCAATAAGTTCCCAACCAATGAACATCAGTGGGTATGAATC
CGCTGTTACCAGCAGAGCCATGCTCCCAGTGAACAGCGAAAGATAGC
TGAAGAATCGTGGTGTGTGTGGGTCTTCAGCCATGTATGACATGCTA
TAGACGTGTACAGCGCTAGATACGCATAGGACAGGTAGAAGCATCGC
AACTGTAAGGTCATCGAAGCTAAATGCCCATTGAGCTTGTAGCACAC
CAACGGTAAGCCAGTCACCTACAACGATAGTAACAGGTGAACGGCAA
AGAGCTACTTCGTAGAAGGCTACTAGTGATAGACATGCCGATAGTAC
TAGGCATACAGTGGCAACTAGTTGTGAACCTGTGCTACCAAGTGCTC
GCCCTCGTAGACCAGCGGCTGCTGAACCTAGGAAGGGTAGAAGAAT
AAGAGCTAGGTACATAGACTATAGTCGTGGAGATGATAGGCTCAGTG
TCCCCCGTAGGCGGTAATAGGCAACTAGTACTCCAAGACCAATAGCG
CTTTCAGCTCCCGCCGTAGCGATGATAATGATACTGAATGTCATTCCA
GCAGCATCGCTATAGCTGTTAGCTGTTGTGAGAACGAGTAGTGTCAC
CGCGAGCAGCATCAGTTCAATGCTCACAAGTAGCATGAGGAGGCTAC
GCCGGTTAAGGCTAAACCCCAGCACACCAACAAGGAACAGTACGAGT
GATAGAGTCATGGGACAGTCATTCGTGATAGGCGGAAGGCCATGTTT
AAAACATCGTACAGTAGGAGTACTTGGCATCCATGATAGTGACCATAT
CACTCACTTCCCCCCCGAAAGGGGGTAGTGGTAAGTAGCAGACTGTT
GCCAAACAGACTACCTATATCAGTAGTATAGTATCAGTACGTGGACCA
GCAACATTGAGTTACGGGCGACCCACTAACTCTCTCCACCCTTTACC
CCCCTATGAGGGGGGGTTGAAGGGATAGGTAAAAGGATTTAGTAAGA
AGGGATGTTTAAAAGATGGGTAAATATATTATTATAAGGTAGGGTTTA
GATTATGTGGGAGATTATTATAAGAATCCCGCGGCAGATGGGACTCG
AACCCACACTCACAACGTTGACAACGTTGGACACTGCCATTATGCTA
CTACCGCTAGGCACTGTCCTATTACTCTAATTACTATCTTTGTGATAAT
ACAGTTGTATTAGTATAGTAACAATAAAAGGTATCATCTGACNNNNNN
NNNNCCTTTTGGTGAAAGGAGAAAAGAAAAATCTATACCGCTGGTAC
GTTGTAGAGCAGTGCAGAAGCTTCAACTTCTAGACCAGACAGTAGTA
CGCCAGGGATCACACCAAGGAATAGAGTCATGACGATCAGAGGTAG
AACTGTCATAGCTTCTGTTCGTGTCAGGTCACCTGCGATTGTTAGGTA
TGGACTTAGAGTTCCTGCGCTGATACGGGCCCATAGCCATGTAGAGT
AGCAAGCGCTAAGCACGATACCTGTAGCTCCAAGAGCACAAGCTAGG
AATGATTGTTCCATCACCCTGCTAGAGCCATGAATTCACCCACCCAGT
TGACTGAGAGAGGAACACCCATGTTAGCACAGATAGCAAAGAAGAAC
ACTACTGTGAATACTGGCATAACGCTAGCCATACCTCGGTAGTAGCT
GATAGACCGTGTGTGGTATCGGTTGTATAGAACACCACCCACCATCA
CGAATAGCGCTGGTGAGATTAGACCGTGCGCTAGTGATAGTAGTAGA
GCACCACTGATACCAATTACGCTGTTACTGAAGAGCCCTAGGACGAC
CACAGCCATGTGCCCTACTGATGAGTATGCAATAAGCTGCTTAGTGT
CTACTGACCGTAGTGTGGCTAGACTAGAGTAGATCAGACTTACAACA
GCAAGTGTTTGTACTAGAGGTGCGTAATAGTCGCAAGCATCAGGTAG
TACTGGTAGTACTAGTCGGTATACTCCATACGTGGCAAGCTTCAGCA
CTGTACCAGCTAGAAGCATAGATCCAGGCAGTGGAGCGTCAGCGTG
AGCACGAGGTAGCCAGATATGGAAAGGCACGATTGGAGTCTTTACCA
TAAGGGCTAGACCAAAGCACACAAACAGGTATCGTTGTACAGCAGGG
TCTAGTGATACGTGTTGTAGCACTGTCATGTCTGTTGTACCTGTGTGG
CTATATAGACCTAGAATACCTAGTAGCATGAATAGAGATCCTGCTAGT
GTATATAGGAATAGAAGTAGGGCTGACCGTACTCGTGTTGGTGAGCC
ACCATAGAGACCAACCATCACGTAAAGAGGCATCAGTACAGCTTCGA
ATGCTACGTAGAAGACGATGTAGTCAGTAGTAGTGAATGCAGCGTTA
ATAAGAGCTGTAGCAAGGAACACAAGAGCCAGTTGGGCCTTTTGTGA
AGCGTGAGGAGTGTCAGCTAGAGCTAGGACCATAGGTTCTACTAGGA
AGCATGAAAGTGTTACTAGTAGCATGCTAGTGTAGTCCACACCAAAG
AGAAGAGGAGCACCAATACCAATAAATTCGTGGTATTCCATAGCTCC
CCAGGGGTCAGTAGCCAGTAGCATCAGACAGACCATGTAACGAAGT
GTTTGTGACGACGCCATGGCCACAGTCATAGTAACGCCACCTGGTTG
TTCTGGATTAGCTCCACCTTCAGTAGGTTGCCATGCCAGGTAGATGG
CGGTGATAATAGGATATGCAATTAGATATGTAATCATAGATTTTATTAG
GGGGGTAGTAGCCTTAGTAGAATGGCCATTTGTCCCTCTACCCTGTT
AGTTAATAAATAAAGCTCTGCTTGAATATTATAGAAGTACTTATTATTA
TGATGATAGAACCGTTTATGGCGATAAACCTATATTATACGTATAGCA
GTAGAGTTCTACTAAACCTTTAACGATAGAGCACAATTTATTTATACCT
ATAACCATAGGGCCCATGTTTATGAAGATAGGTTCTTGTATTATGTTTA
TGGTGATAGAACCTATATATATATATATATACGGTTACTCACCATTGTT
TATAAGAGGTAGCCGGATCGAACGGCTGCTAGCGCTGTGTAAAAGC
GCTGACCTAACCACTAGTCGAACCTCCTTGTACTGTTAACATTATCTG
AGCCTATTAATGATTAGAAAATATACTATTTAGTACCATTAGCATAGCT
AGGAGAATACAGGGATACATGGGATCGAACCACGCCTAAGAGCTTTG
GAGACTCTTGTACTACCACTATACGATATCCCTATCAGGCTTAGGACC
GGACATAATATCTATCATCTTAATAGATAGGGGGGCTATCATGCCTAT
TCACAACAGATCATGATAGTATGATAAGCTATGGGTCAAGCCGCGGG
CTCGAACCGCGCACCTCTCCGACCACAACGGAACGCTCTGCCAAAT
GAGCTAGAATGACCTGTGTTATGGTCATACCAATACACCAGAGGAAT
AATAATGCTTAGAGGCAATAATATATTATTCAATACTAATAGTTTCCTC
TGTTTGCCTCCGCGTGGGATCGAACCACGATTTTAGGTTTACAAGAC
CCACGTTGCACCATTCAACTACAGAGGCTCATAGTGATCGATAAAGA
CCCCATATCTATGACCATTGTAACGCTACTGGGACTTGAACCCAGTCT
ACCTGTGTGAAGGACAGGTGTGCTACCCCTACACCATAGCGTCTACT
ACTGTTATATATGACATACTATGTTCATGAGCACGCGCACATAAAAAT
ATCCCTAATAATATAACACTTGAGCTATGGTAAATAGTGGGGGTCTAT
TATTATGCTACCCATTACTACCCTTTTTAGGTTAGTTAAGGAAAAGTAA
AGCGGANNNNNNNNNNTAGCTAGGAGAATACAGGGATACATGGGAT
CGAACCACGCCTAAGAGCTTTGGAGACTCTTGTACTACCACTATACG
ATATCCCTATCAGGCTTAGGACCGGACATAATATCTATCATCTTAATA
GATAGGGGGGCTATCATGCCTATTCACAACAGATCATGATAGTATGAT
AAGCTATGGGTCAAGCCGCGGGCTCGAACCGCGCACCTCTCCGACC
ACAACGGAACGCTCTGCCAAATGAGCTAGAATGACCTGTGTTATGGT
CATACCAATACACCAGAGGAATAATAATGCTTAGAGGCAATAATATAT
TATTCAATACTAATAGTTTCCTCTGTTTGCCTCCGCGTGGGATCGAAC
CACGATTTTAGGTTTACAAGACCCACGTTGCACCATTCAACTACAGAG
GCTCATAGTGATCGATAAAGACCCCATATCTATGACCATTGTAACGCT
ACTGGGACTTGAACCCAGTCTACCTGTGTGAAGGACAGGTGTGCTAC
CCCTACACCATAGCGTCTACTACTGTTATATATGACATACTATGTTCAT
GAGCACGCGCACATAAAAATATCCCTAATAATATAACACTTGAGCTAT
GGTAAATAGTGGGGGTCTATTATTATGCTACCCATTACTACCCTTTTT
AGGTTAGTTAAGGAAAAGTAAAGCGGANNNNNNNNNNGTAAAGCGG
AGCCAGGACTCGAACCATGGACCTACAGCTCATGAGGCTGCAATGCT
GCCAAATTACACTACCCCGCGATTGATTATGACCATATTATATATAAA
GACAGGTTTATCTCCGTACTCGGACGCGATAGGAGTCGAACCTACAA
CTGGTCATCCCAGATACAGCTAGCAACCGTATACACCTCACCAATAG
TTGTGCACGTCCCGTCCTGTTACACATAGGTGAGTAAGCCTATAATAA
TGGGTCATACTATTAACGATAGGTTAAATTAAATCCTATAACAACGGG
ACATGTTATTAGCGATGTTGTTGTTTTCTTGTTATTCATGATTAGGTTT
GCCAACAATAGTGTGTGGTAAAAAGGGTGAGGGGTCATTAGGTAGTT
GGTATGGGTATTGGACTTATGGGTGTGACTACGCTCTGGACGCATGC
CCATTACACGTGTCACCCCTTTACTATCTTTGATAGTTAATCTTGCGC
AGTAGTCTACTGCGTGTCCATAGCGTTTGAACATCGTTATTAGCAATG
TTCAAACGCGTTGATGCATGTCCATAGCTGGATTCGGGCTTTATATGC
TGGCAGCCCTTATCCATTGGGTGCTTAGCTGCCCTGCGGTGGCTCAT
TAATAGGCACAACAGGTACACCAGAGGTACCCTGACACTGGTCCTTT
CGTACTAGGTGCCATCCTCGAACATGCTAATAAAGCACAGCGGGTAT
AAGCTATACTGCTTCACAGCGTATTGACAATAGTGTTATTCATAATAAA
ACAGTGGCTTTCACCACTGGTCAGACTCTATCATAGGCAGCAAGCTT
GCTACCGTGCAGGATAGTTATAGGCTCTGAGCCTTATAGTCGTTGGA
CGTGATACTTTAGAATAATTCACGCTCCTAGTTGTAAGGTATATACTTC
CAGGATGGTTTCTGCTTTTGGTGATCAATCACCGTTGGGCTCAATTTA
CATTATTCTCAACAAAACTATGACTTAAAACAAAGGGGTCTAGTTTAG
GTCAACTCGAGGACCTAGGCGGTACAGCATAGATTCATGCATATGAG
GTGTAAGCATGGCTTGTAGTGCCTTGTGATGGGCCTTTGTAATCGCA
AGCTTGTATTGCTTTTGTCCATCCTTTCCATAACGGTCAGCACGGACA
GTAGTGGGGATACCCATGGCAGTAATGGCCGCTGCTAGGCGTACGC
AATCTGCATGAGTATAGCTATTTACGTAGATGAATACGGTACTGTTTG
CGATGTCATATGAGCCGTCACTCATGATGAGATGGGCCAGTACAACA
GGCGTCACAAGATCCTTAATCATAGCCGGAACTACCTTGATCCACTC
ACCAGCAGCCCCCATCACATAAAACATATTGTGATATTGGTTGAAAAC
GCCGAGAGAAAGAGTCTTCAGCCGGAATTGTCCAGAGGGTAGTGTA
GTAACCCCCGTTTGGCAGAACTGGCTAAATAGTCCATATACCCAATA
GGCATAATCACGATAAGGGGCCCCAAAAGACCATTCCAGGCGTGTAT
TAGATGTAGCACTTGAACGCGATGCATGGCTATCGCCCAGAAGTTGT
CCAATAACCACTTGGTGTAGGTAATATGGGAGCATTACAATGGATCGT
AGACCCCGAATAATTTCGCTTGTAAAGCGTTGAAGAAGTTGTAGTGTT
GATGTCATAGTTGTTTTTTGTTGATTGAATTAAATATATGTCGCTTAAG
CGAACCCAACTCACGTAGCTCCTTAGACTGCGAACAGCAGTACCCTT
CCAAGCTTGTGCACCTGGAGGATGAGCTGAGTCGACATCGAGGTCG
CAAACAGCACGGGCGATATGAACTCTCGCGTGCTATAACGCTGTTAT
CCCTAGCGTAGCGTTAATCGCCTAGCCCAGAGTGTACCCTGACACGT
CTGGGGTTCACTTGGCCATGCTTGCGCACTTTAAGCACTGCTCAGTG
CTATCAATCAGGCACCCATTCATGTCCATGAACGATAGTAGCCCCTAC
ATGGGCAATACCTTAAAAAAGGCTATTCGGGTTTCCATCCCGGGCAA
GGGTGCCATACGTGGAGGCCTCCGGTACTGTATAGGAGGCGACCGT
CCCAGTCAAACTAGCTGATGACCTATCCTAGCCCATTGCCCGTAAGA
GCGGGGCTTCACTACCACGAATCCTCATGGCGGTGTAATAGTCGAAC
GACATACTTCCCCAGATTCATGTGGCGACCGGCCATCACTAGTAAAG
CTGCATAGGGTCTTCTTGCCCTGCTGTGCTAAAACCGCATCTTCACG
GCTTATTCAGTTTCGCTGAGGGGCCCCCGGAGACAGCGGGTGCGTC
GTGACCCTCTTCATGCGGGACCATAATTAGTGGCCAAGGAATTTCGC
TACCTTAGCACCCTTATGGTTAAGGCGGTCGTTTACCTGAGTTAGGTA
CACGTTGAAGCCCTTAAGCTCTACGCAGTACATTCACTCTAGGCACC
GGACAAGGGACGGCCCCTATACGAGCCACAGCTGGTGCTGGGCATA
GCAGGGACCTGTGTTCTTGGTGCACAGTCGCACACCCCGTTCCACTG
CGTCCAGCCAGGATGTTTAATTTCTAAGTTAAAACTAAGCTTAAAAAAT
AAACTATCCCCATAAGCTGGACACCCTTATAGTAGGACGTACGGGTT
TCTGTTGCCGAGTTCCTTTGGGAGCCCTATCTCTCACTCTGGTGTACT
CCACCACGGGACCTGTGTCGGTCATAGGCACGGTAAACGCACGCAG
CTTCAAGGCATATTTATTATATAGTATGCTCAGCTTGTGTTAACTCATC
GCTTTATACTTAGAGACCGGTTTAACTCAGGATGACTGCCATTGTCTA
TCTGAGACCCACCCCGTTCAAGTGCAAGGTGTCTAGACCTTGTATCG
CTACTCGTGTCGCCATGCTCTATCCACGTTGTCCAGACAATGCTACA
CTGCCCTTCTTTCAACATGGGTGCTCATCTACCCTATAATCTTACTAC
ATCATTATTATTATAGCCCGCACCCGCCCATGTTACAAACCCCTTTGG
GGGTAAGAGCAGGTGAGGCATGTGAACTTTAGACATAAATACCCCTA
TCTTTCATGAAAAAGATAGGTAAATAATATTATATGTTTACAGTTTTAG
TAAGTATTTATAGGCGTTGTGTGGGACGATAGCTGAGACCCGTTTATC
TTCGGCGCACTGGTACTCGGCCACTGGGTTGTTACACCATCGTTAAC
AGGTGGCTACCTCCAGGCACACTGCATGACTGTCATTGTAGCAGCAC
CTCCTTACGTGTCATTTAGCTATATCTGTTTGGGTCCTCGCTCAACGC
TCATGACTGTCTTCATCTAGGCTGCTGACCTTATCGCCTACAGCCTGT
GTACAGGACGTAGTAGTCCCCCTTCCATGGTAAGCTACCCCTGATAG
AGCACGGATGCCCCCCAGAACTGCACTCATTTCCTATAAAACCTCCT
AGATGGTGATCTTATATTAAATAAATACATGGCGGCCTGAACAGTACA
GGGTCCTACCACTCGATGCCTGTCTGACGCCTTAACGTCTTTCGATG
AGAGCCGGCTAGCTCGCTGTTAGATAGGCCTATCACCCCTAGCCTGA
GCTCGTCAGAGTACAATACAACGGACACCTGTTCGGCCTCTGTCATG
GCCATCACACGTCAATCAAGTACTTAAAATACTTGGACGTAAGAGGAC
ACAACAGGCCTGGCCCAAGCTAGATCACAGCGGTTCAGGTCTGGGC
AGGAGGACTAATTCCCCTATTGCACCATAATAAGCACCAATATAGCCT
ATTGACTATAACTGACACATATCACATCATTACCTTTATCAATCATTTC
TATTGTAATGATCCATACCTGATTTCACCAATTTATAGTGTAGCTATAT
CTGACGGTTTATCTGATATAACAACAAACTAAAACTGATGGTCAATGG
CTTATATTGGTAGGAGATGTATGGATAGCAATGGGCGATTTTGTCTGT
CGTTTAGGCTATTGGCCTCGCCCTCCTTCCCAACTCGGCGACCCATT
ATGCAAAAGGTACGCTGTCGGGCGTTACTATGGCGATCCTAGAAACC
CGACCATAGCATTGCAGCCCTTCAGCTGCTTATGGACCATAGCTGCT
TCAGGCTCTATTTCACTGCGGTACAACCGCTCTCTTTCATCGTTCACT
CGCGTTACTATTTTCTATCACTCGGACCACCTATTAGCCGTTGGGAGA
GATCCCAATCACTCTACAAAATGCAGAATGCTCTTTAACACATCAGCA
GTCACACGGGACTTTTACCCTCTATGGTTATCTGGCGTTCCATCCAGT
TCAAACTGTCGATGACATGTTACAGCTACACCTACTTTTCGCTCGCCG
CTACTAAGGAGGTACGGTTGGTTGCCTGTAGGCCCTACTATGATGAT
TCACTTTGGGCCATGGCTATCATTGATTTACCGTGAGGTTGTCATTAA
TTGACCTTTGGACTAGGGCNNNNNNNNNNCAACAAAAGATAGTGTGT
TTCCTACTACTCATTACTAACCCCTTCTAATTAGAATTACCCTAACCCC
ACCAAAGGGGGGTAACTGGTGGGTTAAAAGGTAGGTAAAAGTAATAG
TTACTCCACTAGTCATAATGGTTATAGGTGTAGGTACTCACTCATGAA
ATTATGACCATGAGTGTTCATAAATAACTATGACCATGAGTATAACTA
GTAGTATTGGTGGTTAAAAAGTTTAATGGTAATAGAATATGGCTTATG
CTTATGATGGGTGGGGTCTATAATACATGAATGTACTATGGACACCC
GGGTTTGGTGGTCATAAT.

The entire coding region for the ribosomal genes of the PT22AV strain consists of the following sequence:

(SEQ ID NO: 3)
ACCTGGTTGATCCTGCCAGTAGTCATATGCTTGTCTCAAAGATTAAGC
CATGCATGTCTAAGTATAAACAAATTCATACTGTGAAACTGCGAATGG
CTCATTAAATCAGTTATAGTTTATTTGATGGTATCTTGCTACATGGATA
ACTGTGGTAATTCTAGAGCTAATACATGCTGAAAAGCCCCGACTTCTG
GAAGGGGTGTATTTATTAGATAAAAAACCAATGGGTGCAAGCCCTCTA
TGGTGATTCATAATAACTTCTCGAATCGCATGGCCTTGCGCCGGCGA
TGCTTCATTCAAATATCTGCCCTATCAACTTTCGATGGTAGGATAGGG
GCCTACCATGGTATCAACGGGTAACGGGGGAATTAGGGTTCGATTCC
GGAGAGGGAGCCTGAGAAACGGCTACCACATCCAAGGAAGGCAGCA
GGNNNNNNNNNNATCCCGACACGGGGAGGTAGTGACAATAAATAAC
AATATAGGGCCCTATTGGGTCTTATAATTGGAATGAGTACAATTTAAA
TCCCTTAACGAGGAACAACTGGAGGGCAAGTCTGGTGCCAGCAGCC
GCGGTAATTCCAGCTCCAGTAGCGTATATTAAAGTTGTTGCAGTTAAA
AAGCTCGTAGTCGAACTTCGGGCCTGGCTGGGCGGTCCGCCTTACG
GTGTGTACTGTCCGGCCGGGCCTTACCTCATGGTGAGCCCGTATGC
CCTTTACTGGGTGTGCGGTGGAACCATGAATTTTACCTTGAGAAAATT
AGAGTGTTCAAAGCAGGCATAAGCCCGAATACATTAGCATGGAATAA
TAGAATAGGACGTGCGGTTCTATTTTGTTGGTTTCTAGGATCGCCGTA
ATGATTAATAGGGACGGTCGGGGGCATTAGTATTCAGTTGCTAGAGG
TGAAATTCTTAGATTTACTGAAGACTAACTTCTGCGAAAGCATTTGCC
AAGGACGTTTTCATTGATCAAGAACGAAGGTTAGGGGATCAAAAACG
ATTAGATACCGTTGTAGTCTTAACAGTAAACTATGCCGACTAGGGATC
GGGCCACGTTAATTTCTGACTGGCTCGGCACCTTACGAGNNNNNNNN
NNTTCTGGGGGGAGTATGGTCGCAAGGCTGAAACTTAAAGGAATTGA
CGGAAGGGCACCACCAGGTGTGGAGCCTGCGGCTTAATTTGACTCA
ACACGGGGAAACTCACCAGGTCCAGACATAGTAAGGATTGACAGATT
GATAGCTCTTTCTTGATTCTATGGGTGGTGGTGCATGGCCGTTCTTAG
TTGGTGGAGTGATTTGTCTGGTTAATTCCGATAACGAACGAGACCTTA
ACCTGCTAAATAGTCAGGCCGGCTTCGGCTGGTCGTCGACTTCTTAG
AGGGACTGTCGGCGTTTAGCCGACGGAAGTTTGAGGCAATAACAGG
TCTGTGATGCCCTTAGATGTTCTGGGCCGCACGCGCTACACTGACTG
AGCCAGCGAGTTTATCACCTTAGCCGAGAGGCTTGGGTAATCTTGTG
AAACTCAGTCGTGCTGGGGATAGAGCATTGCAATTATTGCTCTTCAAC
GAGGAATACCTAGTAAGCGTAAGTCATCAACTTGCGTTGATTACGTCC
CTGCCCTTTGTACACACCGCCCGTCGCTACTACCGATTGAATGGCTT
GGTGAGATTTCCGGATTGGCGTTGGGGAGCCGGCAACGGCACCCTT
GGCTGAGAAGCTACTCAAACCTGGTCATTTAGAGGAAGTAAAAGTCG
TAACAAGGTTTCCGTAGGTGAACCTGCGGAAGGATCATTATTGATTG
GTCGAAAGACCTTATCAGATTCTACCACCTCTGTGAACCGTTGACCTC
CGGGTTAATAATCAAACATCAGTGTAACGAACGTAAGAGTATCTTAAC
GAAACAAAACTTTCAACAACGGATCTCTTGGCTCTCGCATCGATGAAG
AACGCAGCGAAATGCGATAAGTAATGTGAATTGCAGAATTCAGTGAAT
CATCGAATCTTTGAACGCACCTTGCGCCTTTTGGTATTCCGAAAGGCA
TGCCTGTTTCAGTGTCATGAAATCTCAATCTAATATGTTTTCTGAACAT
GTTAGACTTGGACTTGGGCGTCTGCCAGTGATGGCTCGCCTCAAATG
ACTTAGTGGAACATCCCACATCAGTGTTAGACGTAATAAGTTTCGTCT
CTCCTTGTGGTGATGACTGCTCAGAACCTGCCATCGCGCACTTTTGA
CTTTGACCTGAAATCAGGTAGGGCTACCCGCTGAACTTAAGCATATC
AATAAGCGGAGGAAAAGAAACTAACAAGGATTCCCTAGTAACGGCGA
GTGAACCGGGAAGAGCTCAAATTTGAAATCTGGCGTGCTCAGTGCGT
CCGAGTTGTAATCTATAGAGGCGTTTTCCGTGCCGGACTGTGTCTAA
GTCCCTTGGAACAGGGTATCAAAGAGGGTGATAATCCCGCACTTGAC
ACAATGACCGGTGCTCTGTGATACGTCTTCTACGAGTCGAGTTGTTT
GGGAATGCAGCTCAAAATGGGTGGTGAGTTCCATCTAAAGCTAAATA
TTGGCGAGAGACCGATAGCGAACAAGTACCGTGAGGGAAAGATGAA
AAGCACTTTGGAAAGAGAGTTAAACAGTACGTGAAATTGTTGAAAGG
GAAACGATTGAAGTCAGTCGTGACTGAGAGGCTCAGCCGGTTCTGCC
GGTGTATTCCCTCAGTCGGGTCAACATCAGTTTTGTTCGGTGGATAA
GGGCAGCTGGAAGGTGGCACCTCCGGGTGTGTTATAGCCAGCTGTC
GCATACATCGAATGAGACTGAGGAATGCAGCTCGCCTTTATGGCGGG
GTTCGCCCACGTCCGAGCTTAGGATGTTGACATAATGGCTTTAAACG
ACCCGTCTTGAAACACGGACCAAGGAGTCTAACATATCTGCGAGTGT
TTGGGTGTCAAACCCAAGCGCGTAATGAAAGTAAACGTAGGAGGGAT
CCGCAAGGAGCACCTTCGACCGATCTGGATCTTCTGTGATGGATTTG
AGTAAGAGCATATATGCTGGGACCCGAAAGATGGTGAACTATGCCTG
AATAGGGCGAAGCCAGGGAAACTCTGGTGGAGGCTCGTAGCGATTC
TGACGTGCAAATCGATCGTCAAATTTGGGTATAGGGGCGAAAGACTA
ATCGAACCATCTAGTAGCTGGTTCCTGCCGAAGTTTCCTCAGGATAG
CAGAAACTCGCATCAGTTTTATGAGGTAAAGCGAATGATTAGAGGAAT
TGGGGACGAAACGTCCTTAACCTATTCTCAAACTTTAAATGTGTAAGA
AGGACTTGTCACTTAATTGGACGAGTCCATGCGAATGAGAGTTTCTA
GTGGGCCATTTTTGGTAAGCAGAACTGGCGATGCGGGATGAACCGAT
CGTGAGGTTAAGGTGCCGGAATACACGCTCATCAGACACCACAAAGG
TGTTAGTTCATCTAGACAGCAGGACGGTGGCCATGGAAGTCGGAATC
CGCTAAGGAGTGTGTAACAACTCACCTGCCGAATGAACTAGCCCTGA
AAATGGATGGCGCTCAAGCGTGTTACCCATACCTCACCGTCGGCGTT
GAAGTGACGCGCCGACGAGTAGGCAGGCGTGGAGGTTTGTGAAGAA
GCCTTGGCAGTGATGCTGGGTGAAACAGCCTCTNNNNNNNNNNAGA
TAGGGAAGCTCCGTTTCAAAGTGCACGCTTATCCGTGCCGCCTATCG
AAAGGGAATCCGGTTAAGATTCCGGAACCAGGATGTGGATCATTGAC
GGCAACGTAAATGAAGTTGGAGACGCTGGCAAGGGCCCCGGGAAGA
GTTCTCTTTTCTCCTTGACCGCTTACGACCTCGAAATCGGATTATCCG
GAGATGAGGTTATATGGCGGGCAGAGCACGACACCTCTGTCGTGTC
CGGTGCGTCCTTGACAGTCCTTGAAAATCCGACGGAACGTATAAGTC
TCACGCCTGGTCGTACTCATAACCGCAGCAGGTCTCCAAGGTGAACA
GCCTCTAGTTGATAGAACAATGTAGATAAGGGAAGTCGGCAAAATAG
ATCCGTAACTTCGGGAAAAGGATTGGCTCTAAGGGTTGGGTACGTCG
GGCCATTGGCGGAACAGAGCTGGACCAGGTCGGACTTGCTGGGGCA
ACCCGGCTGGACTGGCTCGGACCGGCGATGGGATGNNNNNNNNNN
AACTATGACTCTCTTAAGGTAGCCAAATGCCTCGTCATCTAATTAGTG
ACGCGCATGAATGGATTAACGAGATTCCCACTGTCCCTATCTACTATC
TAGCGAAACCACAGCCAAGGGAACGGGCTTGGCAGAATCAGCGGGG
AAAGAAGACCCTGTTGAGCTTGACTCTAGTTTGACATTGTGAAAAGAC
ATGGAGGGTGTAGAATAAGTGGGAGCTTCGGCGCCGGTGAAATACC
ACTACCTCCATCGTTTTTTTACTTATTCAATGAGGCGGAGCTGGGATT
AACGTCCCACCTTTTTGTCTTAAGGTCCTTTACGGGCTGATCCGGGTT
GAAGACATTGTCAGGTGGGGAGTTTGGCTGGGGCGGCACATCTGTT
AAAAGATAACGCAGGTGTCCTAAGGGGGACTCATGGAGAACAGAAAT
CTCCAGTAGAACAAAAGGGTAAAAGTCCCCTTGATTTTGATTTTCAGT
GTGAATACAAACCATGAAAGTGTGGCCTATCGATCCTTTAGTCCCTCG
GAACTCGAGGCTAGAGGTGCCAGAAAAGTTACCACAGGGATAACTG
GCTTGTGGCAGCCAAGCGTTCATAGCGACGTTGCTTTTTGATCCTTC
GATGTCGGCTCTTCCTNNNNNNNNNNGGAGTAACTATGACTCTCTTA
AGGTAGCCAAATGCCTCGTCATCTAATTAGTGACGCGCATGAATGGA
TTAACGAGATTCCCACTGTCCCTATCTACTATCTAGCGAAACCACAGC
CAAGGGAACGGGCTTGGCAGAATCAGCGGGGAAAGAAGACCCTGTT
GAGCTTGACTCTAGTTTGACATTGTGAAAAGACATGGAGGGTGTAGA
ATAAGTGGGAGCTTCGGCGCCGGTGAAATACCACTACCTCCATCGTT
TTTTTTTACTTATTCAATGAGGCGGAGCTGGGATTAACGTCCCACCTT
TTTGTCTTAAGGTCCTTTACGGGCTGATCCGGGTTGAAGACATTGTCA
GGTGGGGAGTTTGGCTGGGGCGGCACATCTGTTAAAAGATAACGCA
GGTGTCCTAAGGGGGACTCATGGAGAACAGAAATCTCCAGTAGAACA
AAAGGGTAAAAGTCCCCTTGATTTTGATTTTCAGTGTGAATACAAACC
ATGAAAGTGTGGCCTATCGATCCTTTAGTCCCTCGGAACTCGAGGCT
AGAGGTGCCAGAAAAGTTACCACAGGGATAACTGGCTTGTGGCAGC
CAAGCGTTCATAGCGACGTTGCTTTTTGATCCTTCGATGTCGGCTCTT
CCTGTCNNNNNNNNNNAGAATTCGGTAAGCGTTGGATTGTTCACCCA
CTAATAGGGAACGTGAGCTGGGTTTAGACCGTCGTGAGACAGGTTAG
TTTTACCCTACTGATGGAGGGTTATCGTAACAGTAATTGAGGGTAGTA
CGAGAGGAACTGCTCATTCAGATAATTGGTATTTGCGCCTGTCCGAT
CGGGCAATGGCGCGAAGCTATCATCTGCTAGATTATGGCTGAACGCC
TCTAAGTCAGAATCTGTACTGGAAACGATGATGTTGGTCCCGCATGT
GTTAGTTGTGTCAAAATAGGCTTCGGCTGTGAACCATATCTGGGCTG
GGTTGTTTGGACGGAAAGGTCCTTGCAGCTTGCTCTTGATTGAAATG
GAATATACGCGGGGGGTGAATCCTTTGCAGACGACTTGAATTGGAAC
NNNNNNNNNNTAGTAGAGTAGCCTTGTTGCTACGATCTACTGAGGCT
AAGCCCTTGT.

The COI1 gene of the PT22AV strain consists of the following nucleotide sequence:

(SEQ ID NO: 4)
ATGGCTAGCCGATGGCTCTTCTCTACAAACGCCAAGGATATTGGTAC
CCTTTATATCCTTTTTGCTGTCTTTACCGGTCTTCTGGGTACTGCGTT
CAGTGTACTTATCCGGATGGAGCTATCAGCTCCTGGTAACCAGTTCC
TTTCTGGTAACCACCACCTCTACAACGTAATTGCTACAACTCACGGTA
TCATGATGATTTACTTCATGGTGGTACCTGCTATGGCTGGGTTCGCTA
ACTACATGGCTCCCGTTCTTATCGGTGCGCCGGACATGGCGTTCCCA
CGGCTGAACAACATCAGCTTCTGGCTACTACCTCCTGCAATCGTTCTT
ATCCTTTCAAGCGTCTTCGTGGAACAAGGTATGGGTAGCGGTTGGAC
TATGTACATGCCTCTAACAGGTGTACAATCACACAGTGGTGGATCAGT
TGATCTAGCTGTATTCAGCCTGCACCTCTCAGGGATTTCATCTCTACT
TGGTGCCATTAACATTATCACTACAATCCTTAACATGCGAGCTCCAGG
GCTACGCCTACACAAGGCACCTCTATTCGTATGGGCGATGCTATCAC
AGTCAGTTATTATTATTCTATGTATTCCAGTTCTAGCCGGAGCTCTTAC
TATGATCCTTACTGACCGTAACTTCAACACATCATTCTTCGACCCTGC
TGGAGGAGGGGACCCTGTCCTATACCAACACCTATTCTGGTTCTTCG
GACACCCTGAAGTATACCTAATGATTATCCCTGGATTTGGTATGGTTA
GCCACGTTGTTTCAACATTCAGTGGAAAGCCTGTTTTCGGTTACCTTG
GTATGGTGTACGCCATTGCCTCTATCGGTGTGCTTGGATTCATTGTAT
GGAGCCACCACATGTACGCCGTAGGGATGGACGTAGACACACGAGC
TTACTTCACAGCTGCATCTATGATTATTGCGGTACCTACAGGTATCAA
GGTCTTCAGTTGGCTAGCGACTGCTTACGGTGGTTCTATCCGACTGC
TAGCACCTATGCTATTCGCTCTAGGATTCGTCGGTCTGTTCACTATTG
GTGGACTGACAGGTGTAGTTCTAGCGAACGCGAGCGTTGACGTAGC
GATGCATGACACATACTACGTTGTGGCTCACTTCCACTACGTGCTTTC
AATGGGTGCCGTATTCAGTATCTTTGCCGGGTTCTACTACTGGGCTC
CAAAGATGTTCGGTAAGATGTACGAAGAACACCTAGCTCAAGCGCAC
TTCTGGACATTCTTCATCGGTGTTAACATGACATTTATGCCACAACAC
TTCCTAGGACTACAAGGAATGCCACGACGATTCGCTGACTACCCTGA
CGCTTACGCGGGATGGAACCTAATCTCATCATGGGGTTCATTCGTTA
GCGTCTCTGCTACTCTTCTGTTCCTGTACACTGTTTACGACATGTTCA
CTAACGACAAGTACGTTGGTGACAACCCATGGGGAGAACCGCTATAC
TTCACGGACAACCTAACGTACGAACGAAGCACACGGTTCGCCTACAC
AATCGAGTGGGTACTACCATCACCTACTCCACACCACGCATTCGACA
TGCTACCTATCCAATCATAG.

The taxonomic classification of the PT22AV strain of Papiliotrema terrestris is shown in Table 1.

### EXAMPLE 2. ANALYSIS OF THE ANTAGONISTIC CAPACITY AND MECHANISMS OF ACTION OF PAPILIOTREMA TERRESTRIS PT22AV

The PT22AV strain was first characterized in vitro by analyzing its antagonistic capacity and mechanisms of action. The assay consisted of a pure culture in which the radial growth of fungal pathogens on an agar growth medium was evaluated and compared to the growth of the same plant pathogens in dual culture with Papiliotrema terrestris PT22AV as biocontrol agent. By evaluating the differences in radial growth of the fungal pathogens in the different conditions, it was possible to quantify the antifungal effect produced by the antagonist yeast. The average inhibition produced by the PT22AV strain against the various fungal pathogens tested is approximately 18%, specifically: Penicillium expansum 25%; Monilinia fructigena and M. laxa 20%; Botrytis cinerea 15%; Fusarium spp. 10%; Rhizoctonia solani 20%; Alternaria solani 15%; Aspergillus spp. 25%; Colletotrichum spp. 20%.

### EXAMPLE 3. EVIDENCE OF EFFICACY OF PAPILIOTREMA TERRESTRIS PT22AV IN COMPARISON WITH KNOWN PRODUCTS

The PT22AV strain of Papiliotrema terrestris species was tested in the context of efficacy trials with GAP certification, according to the guidelines: PP 1/135(4) Phytotoxicity assessment, PP 1/152(4) Design and analysis of efficacy evaluation trials and PP 1/181(5), Conduct and reporting of efficacy evaluation trials, including good experimental practice. In particular, 32 efficacy and selectivity tests conducted over a period of 3 years (2018-2019-2020) in 4 EU countries, on 6 crops, against a wide range of biotrophic and necrotrophic fungal pathogens, in the field and in the post-harvest.

Tables 3a and 3b shows the efficacy of Papiliotrema terrestris PT22AV applied in the field or post-harvest against areal part fungal pathogens (Table 3a), and as soil inoculant and/or in fertigation against soil-borne fungal pathogens (Table 3b), in different formulations (WG: wettable granule—WP: wettable powder—SC: suspension concentrated) and at different dosages.

The WG and WP formulations have the following composition: dehydrated yeast cells, cryoprotectants (mono and disaccharides), inert substances of mineral or organic nature, disintegrants. The quantitative ratios of the different components that make up the formulate vary according to the microbial concentration of the formulate. The SC formulate has the following composition: yeast cells, water, stabilizers.

The effectiveness of the PT22AV strain was compared with that of biological and chemical competitors using the following formula:

{[(Competitor efficacy×100)/PT22AV efficacy]−100}×−1.

In this formula, the efficacy of PT22AV is considered as the 100% and the percentage of fungicide competitor's efficacy is calculated in the first step. Subsequently, the percentage difference between the two treatments is calculated (−100). Finally, by multiplying for −1 the result will acquire a positive sign in the comparisons showing that PT22AV has a higher efficacy and a negative one in the comparisons where PT22AV displays a lower efficacy.

TABLE 3a
Efficacy comparison between PT22AV formulations and fungicide competitors (areal part fungal pathogens).

| | | | | Treatment | | | | | |
|---|---|---|---|---|---|---|---|---|---|
| | | | | (CFU per | Efficacy | Efficacy compared to known Competitor | | | |
| Host plant | Pest | Disease | Formulation | ha) | (%) | products (%) | | | |
| Grapevine | Erysiphe | powdery | WG | 2.0 × 10¹² | 80 | Bacillus subtilis | +12 | Sulfur | +18 |
| | necator | mildew | WP | 2.5 × 10¹² | 90 | | +22 | | +27 |
| | | | WP | 1.2 × 10¹² | 88 | | +20 | | +26 |
| | | | WP | 2.0 × 10¹² | 95 | | +26 | | +31 |
| | Botrytis | Grey mold | WG | 2.0 × 10¹² | 82 | | +14 | Cyprodinil | −3 |
| | cinerea | (in field) | WP | 2.5 × 10¹² | 88 | | +20 | Fludioxonil | +3 |
| | | | WP | 1.2 × 10¹² | 75 | | +7 | | −13 |
| | | | WP | 2.0 × 10¹² | 92 | | +13 | | +7 |
| | | | WP | 9.0 × 10¹¹ | 40 | | −30 | | −112 |
| | | | SC | 2.0 × 10¹² | 87 | | +20 | | +2 |
| | | Grey mold | WG | 2.0 × 10¹² | 33 | | +40 | | +15 |
| | | (post-harvest) | WP | 2.5 × 10¹² | 25 | | +20 | | −12 |
| | | | WP | 1.2 × 10¹² | 22 | | +10 | | −27, |
| | | | WP | 2.0 × 10¹² | 70 | | +71 | | +60 |
| | | | WP | 9.0 × 10¹¹ | 20 | | +8 | | −40 |
| | | | SC | 2.0 × 10¹² | 81 | | +13 | | +65 |
| Strawberry | Botrytis | Grey mold | WP | 2.5 × 10¹² | 55 | Bacillus subtilis | +18 | Cyprodinil | −29 |
| | cinerea | (in field) | WG | 2.0 × 10¹² | 68 | | +33 | Fludioxonil | −4 |
| | | | WG | 2.0 × 10¹² | 58 | | +22 | | −22 |
| | | | WP | 2.5 × 10¹² | 78 | | +42 | | +8 |
| | | | WP | 1.2 × 10¹² | 57 | | +21 | | −24 |
| | | | WP | 2.0 × 10¹² | 85 | | +47 | | +16 |
| | | | WP | 9.0 × 10¹¹ | 42 | | −7 | | −69 |
| | | | SC | 2.0 × 10¹² | 88 | | +48 | | +19 |
| | | Grey mold | WP | 2.5 × 10¹² | 47 | | +14 | | −42 |
| | | (post-harvest) | WG | 2.0 × 10¹² | 56 | | +28 | | −19 |
| | | | WG | 2.0 × 10¹² | 49 | | +22 | | −36 |
| | | | WP | 2.5 × 10¹² | 80 | | +50 | | +16 |
| | | | WP | 1.2 × 10¹² | 61 | | +34 | | −9 |
| | | | WP | 2.0 × 10¹² | 88 | | +54 | | +23 |
| | | | WP | 9.0 × 10¹¹ | 45 | | +11 | | −48 |
| | | | SC | 2.0 × 10¹² | 78 | | +48 | | +14 |
| Potato | Phytophthora | Potato blight | WP | 2.5 × 10¹² | 45 | Copper | +51 | | |
| | infestans | | WG | 2.0 × 10¹² | 61 | oxychloride | +63 | | |
| | | | WG | 2.0 × 10¹² | 46 | | +52 | | |
| | | | WP | 2.5 × 10¹² | 76 | | +71 | | |
| | | | WP | 1.2 × 10¹² | 43 | | +48 | | |
| | | | WP | 2.0 × 10¹² | 80 | | +72 | | |
| Apple tree | Stemphylium | Brown spot | WG | 2.0 × 10¹² | 87 | Aerobasidium | +64 | Fludioxonil | +25 |
| | sp. | | WG | 2.0 × 10¹² | 80 | pullulans | +70 | | +18 |
| | | | WG | 3.0 × 10¹² | 88 | | +63 | | +26 |
| | | | WP | 2.5 × 10¹² | 80 | | +70 | | +18 |
| | | | WP | 1.2 × 10¹² | 77 | | +72 | | +15 |
| | | | WP | 3.0 × 10¹² | 90 | | +62 | | +27 |
| | | | WP | 9.0 × 10¹¹ | 68 | | +54 | | +5 |
| | | | SC | 2.0 × 10¹² | 79 | | +60 | | +17 |
| | Necrotrophic | Post-harvest | WG | 2.0 × 10¹² | 79 | Aerobasidium | +36 | Fludioxonil | +25 |
| | fungi | rot (field | WG | 2.0 × 10¹² | 54 | pullulans | −1 | | −9 |
| | | treatment) | WG | 3.0 × 10¹² | 87 | | +36 | | +32 |
| | | | WP | 2.5 × 10¹² | 72 | | +23 | | +18 |
| | | | WP | 1.2 × 10¹² | 67 | | +17 | | +12 |
| | | | WP | 2.0 × 10¹² | 96 | | +42 | | +40 |
| | Necrotrophic | Post-harvest | WG | 100 gr/100 l | 75 | Aerobasidium | +66 | Fludioxonil | +17 |
| | fungi | rot (post-harvest | WP | 50 gr/100 l | 80 | pullulans | +68 | | +22 |
| | | treatment) | | | | | | | |
| Peach tree | Monilia spp. | Brown rot | WG | 2.0 × 10¹² | 82 | Bacillus subtilis | +18 | Cyprodinil | −8 |
| | | | WG | 2.0 × 10¹² | 70 | | +5 | Fludioxonil | −27 |
| | | | WG | 3.0 × 10¹² | 91 | | +26 | | +3 |
| | | | WP | 2.5 × 10¹² | 86 | | +22 | | −2 |
| | | | WP | 1.2 × 10¹² | 73 | | +9 | | −21 |
| | | | WP | 2.0 × 10¹² | 88 | | +23 | | 0 |
| | | | WP | 9.0 × 10¹¹ | 60 | | −11 | | −46 |
| | | | SC | 4.0 × 10¹² | 85 | | +21 | | −3 |
| Tomato | Botrytis | Grey mold | WP | 2.5 × 10¹² | 40 | Bacillus subtilis | −10 | Cyprodinil | −37 |
| | cinerea | | WG | 2.0 × 10¹² | 62 | | +30 | Fludioxonil | +11 |
| | | | WG | 2.0 × 10¹² | 57 | | +22 | | +3 |
| | | | WP | 2.5 × 10¹² | 79 | | +44 | | +30 |
| | | | WP | 1.2 × 10¹² | 63 | | +30 | | +12 |
| | | | WP | 2.0 × 10¹² | 82 | | +46 | | +32 |
| | | | WP | 9.0 × 10¹¹ | 51 | | +13 | | −7 |
| | | | SC | 2.0 × 10¹² | 88 | | +50 | | +37 |

TABLE 3b
Efficacy comparison between PT22AV formulations
and fungicide competitors (soil-borne pathogens).
Soil single application

| | | | | Efficacy |
|---|---|---|---|---|
| | | Application | | compared to |
| | | rate PT22AV | Efficacy | Trichoderma |
| Host crop | Pest | (CFU/g soil) | PT22AV (%) | viride (%) |
| Tomato | Rhizoctonia solani | 1 × 10⁷ | 78 | +17 |
| | Fusarium oxysporum | 1 × 10⁷ | 74 | −5 |
| | Pythium ultimum | 1 × 10⁸ | 75 | +7 |
| Lettuce | Sclerotinia sclerotiorum | 1 × 10⁷ | 71 | −13 |
| | Fusarium oxysporum | 1 × 10⁷ | 73 | +4 |
| | Pythium ultimum | 1 × 10⁸ | 79 | +6 |
| Melon | Fusarium oxysporum | 1 × 10⁷ | 87 | +16 |
| | Pythium ultimum | 1 × 10⁸ | 81 | −4 |
| | Rhizoctonia solani | 1 × 10⁸ | 82 | +10 |
| Aubergine | Phytophthora capsici | 1 × 10⁷ | 72 | +13 |
| | Rhizoctonia solani | 1 × 10⁸ | 85 | +18 |
| | Pythium ultimum | 1 × 10⁸ | 78 | +6 |

Soil application + Irrigation

| | | | | Efficacy |
|---|---|---|---|---|
| | | Application | | compared to |
| | | rate PT22AV | Efficacy | Trichoderma |
| Host crop | Pest | (CFU/ml) | PT22AV (%) | viride (%) |
| Tomato | Rhizoctonia solani | 5 × 10⁶ CFU | 84 | +23 |
| | Fusarium oxysporum | | 87 | +10 |
| | Pythium ultimum | | 88 | +20 |
| Lettuce | Sclerotinia sclerotiorum | | 91 | +12 |
| | Fusarium oxysporum | | 85 | +18 |
| | Pythium ultimum | | 85 | +13 |
| Melon | Fusarium oxysporum | | 92 | +21 |
| | Pythium ultimum | | 93 | +10 |
| | Rhizoctonia solani | | 90 | +18 |
| Aubergine | Phytophthora capsici | | 82 | +23 |
| | Rhizoctonia solani | | 88 | +20 |
| | Pythium ultimum | | 87 | +16 |

### EXAMPLE 4. COMPATIBILITY OF PAPILIOTREMA TERRESTRIS PT22AV WITH FUNGICIDES AND EFFICACY IN PROTOCOLS OF INTEGRATED DISEASE MANAGEMENT

The microbial formulates based on Papiliotrema terrestris strain PT22AV can be effectively applied in integrated pest management (IPM) in combination with synthetic chemical active ingredients. This possibility arises from the ability of the PT22AV yeast to tolerate the active ingredients with fungicidal action, allowing the two formulations to be used simultaneously or in succession in time (Table 4).

The resistance or tolerance of the yeast object of the present invention was established following two main approaches:
i) The synthetic chemical active principles were incorporated, at increasing concentrations, in an agarized growth medium (NYDA: Nutrient broth 8 gr l⁻¹; Yeast extract 5 gr l⁻¹; Glucose 10 gr l−1; Agar 20 gr |⁻¹). Cell suspensions of the PT22AV strain, suitably diluted, were distributed on the Petri dishes containing the growth medium with the antifungal molecules, so the microorganism growth in the presence of fungicides was compared to that in the negative control (NYDA without fungicides).
ii) The active ingredients tested were dissolved in sterile distilled water at the concentrations suggested by the manufacturer. The PT22AV yeast-based formulates were suspended in these solutions and incubated at 20° C. with stirring at 150 rpm for 1 hour. The yeast suspension in distilled water was used as the negative control. The suitably diluted cell suspensions were inoculated on growth agar medium (NYDA) to verify their viability.

The first test method highlighted a good level of resistance of the PT22AV yeast to most of the fungicides tested, although with different tolerance levels among the various active ingredients tested (Table 4).

The second test method allowed to establish tolerance to synthetic chemical active ingredients in tank mix conditions, assuming a residence time in the spraying machine tanks for one hour. In this case, no tested fungicide caused a reduction in the viability of the PT22AV yeast as compared to the negative control. The broad spectrum of resistance that emerged is due to the fact that the yeast is placed in a state of quiescence determined by the formulation process and the cell activation time, following rehydration, is approximately 2 hours. In this period of time, the yeast cells resist better to abiotic stress than those in active growth; this allows the simultaneous use of the yeast with all the antifungal active ingredients.

TABLE 4
Minimum inhibitory concentration of
different fungicides against PT22AV

| | | | |
|---|---|---|---|
| | | Minimum | |
| | Al concentration | inhibitory | |
| Active Ingredient | in the commercial | concentration | Resistance |
| (AI) | formulation | (mg/l) | level |

Strobilurins

| | | | |
|---|---|---|---|
| Azoxystrobin | 22.9% | 1000 | Resistant |
| Trifloxystrobin | 50% | 1000 | Resistant |

Benzimidazoles

| | | | |
|---|---|---|---|
| Benomyl | 50% | 1000 | Resistant |
| Carbendazim | 41.7% | 1000 | Resistant |
| Thiabendazole | 41.8% | 1000 | Resistant |

Sterol inhibitors

| | | | |
|---|---|---|---|
| Tebuconazole | 25% | 100 | Sensitive |
| Imazalil | 10% | 300 | Sensitive |
| Penconazol | 10.2% | 200 | Sensitive |

Dicarboximide

| | | | |
|---|---|---|---|
| Procymidone | 50% | 1000 | Resistant |
| Vinclozolin | 50% | 1000 | Resistant |

Dithiocarbamates

| | | | |
|---|---|---|---|
| Mancozeb | 50% | 300 | Sensitive |
| Ziram | 81% | 500 | Resistant |

Inorganic fungicides

| | | | |
|---|---|---|---|
| Copper oxychloride | 40% | 500 | Resistant |
| Sulfur | 80% | 2000 | Resistant |

Phenylpyrrole

| | | | |
|---|---|---|---|
| Fludioxonil | 50% | 1000 | Resistant |

Fungicides assembled

| | | | |
|---|---|---|---|
| Iprovalicarb + | 4.2% + 35.6% | 500 | Resistant |
| Copper oxychloride | | | |

### EXAMPLE 5. BIOSTIMULANT ACTIVITY BY THE YEAST SPECIES P. TERRESTRIS, IN PARTICULAR STRAIN PT22AV, ON CROP PLANTS

According to the present invention, for the first time biostimulant activity by the yeast species P. terrestris, in particular strain PT22AV, on crop plants has been shown.

The PT22AV strain has been applied via root application on melon, strawberry and lettuce crops as indicated in Table 2 and the biostimulant activity of the strain was assessed.

The results, reported in Table 5, show that the radical application of microbial formulations based on P. terrestris strain PT22AV increases the biomass of all the three crops assayed (melon, strawberry and lettuce), compared to the untreated check. The stimulation produced by the roots application of the yeast PT22AV is also evident in terms of leaf surface and the number of leaves/flowers.

TABLE 5

| | | | | | |
|---|---|---|---|---|---|
| | | | | Dry | Dry |
| | Leaf | | | weight of | weight of |
| | surface | Number | Number | the root | the aerial |
| Treatments | (cm²) | of leaves | of flowers | system (gr) | part (gr) |

Crop: MELON

| | | | | | | |
|---|---|---|---|---|---|---|
| 1 | Untreated | 179 | 8.25 | 4.5 | 2.9 | 1.5 |
| | control | (a) | (a) | (a) | (a) | (a) |
| 2 | PT22AV | 451.75 | 14 | 7.25 | 4.45 | 2.3 |
| | WP | (c) | (b) | (b) | (b) | (b) |
| 3 | PT22AV | 421.75 | 14 | 7.30 | 4.2 | 2.35 |
| | WG | (c) | (b) | (b) | (b) | (b) |
| 4 | Radifarm | 299.75 | 12.75 | 7.75 | 3.77 | 2.12 |
| | | (b) | (b) | (b) | (ab) | (b) |

Crop: STRAWBERRY

| | | | | | | |
|---|---|---|---|---|---|---|
| 1 | Untreated | 344 | 9 | 2.25 | 2.42 | — |
| | control | (a) | (a) | (a) | (a) | |
| 2 | PT22AV | 868 | 17.75 | 7.75 | 4.3 | — |
| | WP | (c) | (c) | (c) | (b) | |
| 3 | PT22AV | 811.75 | 17.25 | 7.5 | 4.17 | — |
| | WG | (c) | (c) | (c) | (b) | |
| 4 | Radifarm | 508.5 | 14 | 4.75 | 3.85 | — |
| | | (b) | (b) | (b) | (b) | |

Crop: LETTUCE

| | | | | | | |
|---|---|---|---|---|---|---|
| 1 | Untreated | — | 12.6 | — | 3.32 | 1.47 |
| | control | | (a) | | (a) | (a) |
| 2 | PT22AV | — | 18.75 | — | 4.45 | 2.25 |
| | WP | | (b) | | (b) | (b) |
| 3 | PT22AV | — | 19.7 | — | 4.62 | 2.2 |
| | WG | | (b) | | (b) | (b) |
| 4 | Radifarm | — | 17.72 | — | 4.2 | 2.07 |
| | | | (b) | | (b) | (b) |

WP = Wettable powder,
WG = Water dispersible granules
The values marked with the same letters are not statistically different (at P ≤ 0.05) according to Tuckey’s test.

### EXAMPLE 6. EFFICACY COMPARISON BETWEEN PT22AV AND LS28 ISOLATES IN THREE REPRESENTATIVE CROPS

The efficacy of PT22AV and LS28 strains was compared in in-field and post-harvest trials that were carried out for a period 5 years (from 2016 to 2020) in Italy, at the Molise University (Plant Pathology laboratory, Department of Agricultural, Environmental and Food Sciences), on 7 crops in field trials and on 4 crops in post-harvest trials, against a wide range of biotrophic and necrotrophic fungal pathogens.

PT22AV and LS28 were applied following the protocol described in Table 2. For the field trials the efficacy was evaluated at harvest, while in the post-harvest the measurements were performed when in the untreated control at least 10% of infection was obtained. At the same level of CFU (Colony Forming Units) per hectare, significantly higher efficacy of strain PT22AV than strain LS28 was observed. The comparison of application protocols between strains PT22AV and LS28, with reference to dosage, crop plants and relevant plant pathogens are shown in Table 6.

TABLE 6
Efficacy comparison between PT22AV and LS28
isolates in three representative crops

| | | | | |
|---|---|---|---|---|
| | | | | Efficacy |
| | | | CFU | PT22AV vs |
| Culture | Disease | Pathogen | per ha | LS28 (%) |

Field trials

| | | | | |
|---|---|---|---|---|
| Stone-fruit | Brown rot | Monilia spp. | 1 × 10¹² | +17% |
| | Gray mold | Botrytis | | +22% |
| | | cinerea | | |
| Grape | Powdery | Erysiphe | 2 × 10¹² | +29% |
| | mildew | necator | | |
| | Gray mold | Botrytis | 1 × 10¹² | +32% |
| | | cinerea | | |
| | Downy | Plasmopara | 2 × 10¹² | +41% |
| | mildew | viticola | | |
| | Bunch rot | Aspergillus | | +17% |
| | | spp. | | |
| | Acid rot | yeasts and | | +15% |
| | | bacteria | | |
| Strawberry | Gray mold | Botrytis | 1 × 10¹² | 19% |
| | | cinerea | | |

Post-harvest trials

| | | | | |
|---|---|---|---|---|
| Stone-fruit | Brown rot | Monilia spp | 5 × 10⁷ | +18% |
| | Gray mold | Botrytis | | +10% |
| | | cinerea | | |
| Grape | Gray mold | Botrytis | | +32 |
| | | cinerea | | |
| | Bunch rot | Aspergillus | | +12% |
| | | spp. | | |
| | Acid rot | Yeast and | | +45% |
| | | bacteria | | |
| Strawberry | Gray mold | Botrytis | | +31% |
| | | cinerea | | |

Analogous comparisons of the Applicant have shown a significantly greater ability to resist a wide range of abiotic stresses by PT22AV as compared to LS28 (Table 7). This greater resistance to stressors is associated with higher adaptability, survival and viability in the environment, and with the colonization of plant tissues; these traits are in turn intuitively linked to the biocontrol activity against plant pathogens. Furthermore, greater resistance to stressors is associated with greater resistance and survival during the stage of yeast biomass production and to the stresses due to dehydration and freeze-drying methods, common steps of the formulation process, thus ensuring the production of a greater quantity of vital and active yeast biomass for biocontrol. In particular, the biomass production process results in a CFU concentration of strain PT22AV that is on average 70% higher than strain LS28. In addition, the successive formulation step further reduces the CFU concentration of LS28 of about 40% with respect to PT22AV strain with freeze dry process and over 90% with fluid bed formulation.

TABLE 7
Effect of stress on PT22AV and LS28 growth on agarized media.

Tolerance level

| Stress type | Compound | PT22AV | LS28 |
|---|---|---|---|
| Oxidative | H₂O₂ | +++ | + |
| | Menadione | ++ | − |
| | Patulin | +++ | + |
| | Sodium hypochlorite | +++ | ++ |
| | Cumene hydroperoxide | + | − |
| Genotoxic | UV radiation | +++ | ++ |
| | Hydroxyurea | + | − |
| Cell wall | Red congo | +++ | ++ |
| stress | SDS | +++ | +++ |
| | Calcofluor white | ++ | − |
| | Caffeine | ++ | + |
| Osmotic | NaCl | +++ | ++ |
| stress | KCl | +++ | + |
| | Lithium chlorite | +++ | ++ |

The symbol (+++) stands for highly tolerant, (++) moderately tolerant, (+) low tolerant and (−) sensitive

### EXAMPLE 7. PT22AV EFFICACY AGAINST NEMATODES (MELOIDOGYNE SPP.) ON TOMATO PLANTS

The PT22AV strain has been applied on tomato plants as described in Table 2 and the efficacy of the strain against Meloidogyne spp nematodes was assessed. The results, reported in Table 8, show that when PT22AV strain is applied as a root treatment, while partially reducing the proliferation of nematodes it is also especially effective in limiting their harmful effects, as shown by measuring plant height.

TABLE 8
PT22AV efficacy against nematodes (Meloidogyne spp.) on tomato plants

| | Application timing | | | |
|---|---|---|---|---|
| | A: drenching before transplantation; BCD: through | | | |
| | irrigation, with 7 days-time interval from each other | | | |
| | | | % efficacy | |
| | Active | Application rate | (Abbot | Average |
| Treatment | substance | (g or mL/ha) | transformation) | Height © |
| Untreated | — | — | 0.00 b | 70.68 ab |
| Control | Papiliotrema | A: 1000 g/100 L | 14.89 b | 76.10 a |
| PT22AV | terrestris | BCD: 1000 g/ha | | |
| | 3.5 × 10⁹ | | | |
| | CFU/g | | | |
| PT22AV | Papiliotrema | A: 500 g/100 L | 10.11 b | 74.60 ab |
| | terrestris | BCD: 500 g/ha | | |
| | 3.5 × 10⁹ | | | |
| | CFU/g | | | |
| AV-YD-20-01 | Yeast | 10 l/ha | 1.18 b | 67.30 ab |
| | Derivatives 20% | | | |
| BIOACT | Paecilomyces | A: 750 ml/ha | 3.81 b | 65.08 ab |
| | lilacinus | BCD: 750 ml/ha | | |
| ERGOFERT | Yeast fluid | A: 6000 ml/ha | 3.72 b | 63.83 b |
| NEMACONTROL | extract 42.29% | BCD: 3000 ml/ha | | |
| VELUM PRIME | Fluopyram | 625 ml/ha | 47.06 a | 74.45 ab |
| | 400 g/L | | | |

The values marked with the same letters are not statistically different (at P < 0.05) according to Tuckey's test.

The present invention has been described for illustrative, but not limitative purposes, according to its preferred embodiments, but it is to be understood that variations and/or modifications may be made by those skilled in the art without thereby departing from the relative scope of protection, as defined from the attached claims.

1. Yeast strain belonging to Papiliotrema terrestris species, said strain being deposited at the Westerdijk Fungal Biodiversity Institute with deposit number CBS147138.

2. Yeast strain according to claim 1, wherein said yeast strain is in a form selected from fresh cells, dried cells, dehydrated cells, devitalized cells, inactivated cells, frozen cells or cells in aqueous suspension.

3. Yeast strain according to claim 2, wherein said yeast strain is in the form of cells in an aqueous suspension that includes extracellular polysaccharides of the cell biomass production process.

4. A composition comprising the yeast strain of the Papiliotrema terrestris species, as defined in claim 1, as active principle, together with one or more phytopharmacologically acceptable excipients or adjuvants.

5. The composition according to claim 4, wherein said composition is in liquid or solid form.

6. The composition according to claim 4, wherein the concentration of said yeast strain within the composition ranges from 10³ CFU to 10¹³ CFU per gram of solid composition or from 10² CFU to 10¹² CFU per mL of liquid composition.

7. The composition according to claim 4, wherein said composition is in the form of tablets, capsules, granules, pellets, powder, such as dry powder or wettable powder, fluid, dry fluid, emulsion, suspension, solution, dispersion.

8. The composition according to claim 4, said composition further comprising one or more chemical compounds and/or one or more biological agents selected from fungicides, insecticides, fertilizers, plant biostimulants, macronutrients, micronutrients, pathogen-resistance inducers, plant growth regulators, foliar nutrients, antibiotics, herbicides, acaricides, food additives, microorganisms.

9. The composition according to claim 8, wherein said fungicides are selected from 2-methoxyethylmercury chloride, 2-phenylphenol, 3-ethoxypropyl mercury bromide, 8-hydroxyquinoline sulfate, 8-phenylmercurioxyquinoline, acibenzolar, acibenzolar-S-methyl, acylamino acid fungicides, acypetacs, Adavelt, Agrobacterium radiobacter K84, aldimorph, allyl alcohol, ametoctradin, aminopyrifen, amisulbrom, ampropylfos, anilazine, aureofungin, azaconazole, azithiram, azoxystrobin, Bacillus amyloliquefaciens (formerly subtilis) strain QST713, Bacillus amyloliquefaciens strain AH2, strain IT-45, strain FZB24, strain MBI600, strain D747, Bacillus mycoides isolate J, Bacillus nakamurai strain F727, Bacillus pumilus strain QST 2808, Bacillus subtilis var. amyloliquefaciens strain FZB24, Bacillus subtilis strain AFS032321, strain GB03, strain IAB/BS03, barium polysulfide, benalaxyl-M (=kiralaxyl), benodanil, benomyl, benquinox, bentaluron, benthiavalicarb, Benthiavalicarb-isopropyl, benzalkonium chloride, benzamacril, benzamide fungicides, benzamorf, benzohydroxamic acid, benzovindiflupyr, bethoxazin, binapacryl, biphenyl, bitertanol, bithionol, bixafen, blasticidin-S, Bordeaux mixture, boric acid, boscalid, bromuconazole, bupirimate, Burgundy mixture, buthiobate, calcium polysulfide, captafol, captan, carbamorph, carbendazim, carboxin, carpropamid, carvone, cell walls of Saccharomyces cerevisiae strain LAS117, Cheshunt mixture, chinomethionat, chlobenthiazone, chloraniformethan, chloranil, chlorfenazole, chlorodinitronaphthalene, chloroneb, chloropicrin, Chlorothalonil, chlorquinox, chlozolinate, ciclopirox, climbazole, Clonostachys rosea strain CR-7, Coniothyrium minitans, copper hydroxide, copper naphthenate, copper oleate, copper oxychloride, copper soap, copper sulfate, basic, copper zinc chromate, copper(II) acetate, copper(II) carbonate, basic, copper(II) sulfate, coumoxystrobin, cufraneb, cuprobam, cuprous oxide, cyazofamid, cyclafuramid, cyclobutrifluram, cycloheximide, cyflufenamid, cymoxanil, cypendazole, cyproconazole, cyprodinil, Cyprofuram, dazomet, DBCP, debacarb, decafentin, dehydroacetic acid, dichlobentiazox, dichlorophen, dichlorophenyl, dichlozoline, diclobutrazol, diclocymet, diclomezine, dicloran, diethofencarb, diethyl pyrocarbonate, difenoconazole, diflumetorim, dimethachlone, dimethirimol, dimethomorph, dimoxystrobin, diniconazole, diniconazole-M, dinobuton, dinocap, dinocap-4, dinocap-6, dinocton, dinopenton, dinosulfon, dinoterbon, diphenylamine, Dipymetitrone, dipyrithione, disulfiram, ditalimfos, dithianon, DNOC, dodemorph, dodicin, dodine, donatodine, drazoxolon, edifenphos, enestrobin, enestroburin, enoxastrobin, epoxiconazole, etaconazole, etem, ethaboxam, ethirimol, ethoxyquin, ethylene oxide, ethylmercury 2,3-dihydroxypropyl mercaptide, ethylmercury acetate, ethylmercury bromide, ethylmercury chloride, ethylmercury phosphate, etridiazole, extract from Melaleuca alternifolia (tea tree), extract from Reynoutria sachalinensis (giant knotweed), extract from the cotyledons of lupine plantlets (“BLAD”), extract of Swinglea glutinosa, F500, famoxadone, fenamidone, fenaminosulf, fenaminstrobin, fenamistrobin, fenapanil, fenarimol, fenazaquin, fenbuconazole, fenfuram, fenhexamid, fenitropan, fenoxanil, fenpiclonil, fenpicoxamid, fenpropidin, fenpropimorph, fenpyrazamine, fentin, fentin acetate, fentin chloride, fentin hydroxide, ferbam, ferimzone, Florylpicoxamid, fluazaindolizine, fluazinam, fludioxonil, flufenoxystrobin, fluindapyr, flumorph, fluopicolide, fluopimomide, fluopyram, fluoroimide, fluotrimazole, fluoxapiprolin, fluoxastrobin, fluquinconazole, flurozolamide, flusilazole, flusulfamide, Flutianil, flutolanil, flutriafol, fluxapyroxad, folpet, fosetyl, fosetyl-Al, fthalide (phthalide), fuberidazole, furalaxyl, furametpyr, furcarbanil, furfural, furmecyclox, furophanate, Gliocladium catenulatum J1446, Gliocladium virens GL-21, glyodin, griseofulvin, guazatine, halacrinate, hexachlorobenzene, hexachlorophene, hexaconazole, hexylthiofos, hymexazol, imazalil, imibenconazole, iminoctadine, Inatreq (fenpicoxamid), inorganic oils, inpyrfluxam, iodocarb, ipconazole, Ipflufenoquin, iprobenfos, iprodione, iprovalicarb, isofetamid, isoflucypram, isopropanol azole, isoprothiolane, isopyrazam, isotianil, isovaledione, Jun Si Qi, kasugamycin, kresoxim-methyl, laminarin, Lime sulfur (lime sulphur), mancopper, mancozeb, mandestrobin, mandipropamid, maneb, mebenil, mecarbinzid, mefenoxam, mefentrifluconazole, mepanipyrim, mepronil, meptyldinocap, metalaxyl, metalaxyl-M (=mefenoxam), metam, metazoxolon, metconazole, methasulfocarb, methfuroxam, methyl bromide, methyl isothiocyanate, methylmercury benzoate, methylmercury dicyandiamide, methylmercury pentachlorophenoxide, metiram, metominostrobin, metrafenone, metsulfovax, metyltetraprole, milneb, myclobutanil, myclozolin, N-(ethylmercury)-p-toluenesulfonanilide, nabam, naftifine, natamycin, neem oil, nitrothal-isopropyl, nuarimol, octhilinone, ofurace, organic oils, orthophenyl phenol, orysastrobin, oxadixyl, oxathiapiprolin, oxazosulfyl, oxine copper, oxolinic acid, oxpoconazole, oxycarboxin, oxytetracycline, PCNB, pefurazoate, penconazole, pencycuron, penflufen, pentachlorophenol, penthiopyrad, phenamacril, phenylmercuriurea, phenylmercury acetate, phenylmercury chloride, phenylmercury nitrate, phosdiphen, Phosphite, phosphorous acid and salts, phosphorus acid, phthalide, picarbutrazox, picolinamides, picoxystrobin, piperalin, plant oils (mixutures): eugenol, geraniol, thymol, polycarbamate, polyoxin-D, potassium azide, potassium bicarbonate, potassium polysulfide, potassium thiocyanate, probenazole, prochloraz, procymidone, propamocarb, propiconazole, propineb, proquinazid, prothiocarb, prothioconazole, Pseudomonas chlororaphis strain AFS009, Pseudomonas syringae ESC-10, pydiflumetofen, pyracarbolid, pyraclostrobin, pyrametostrobin, pyraoxystrobin, pyrapropoyne, pyraziflumid, pyrazophos, pyribencarb, pyributicarb, pyridachlometyl, pyridinitril, pyrifenox, pyrimethanil, pyrimorph, pyriofenone, pyrisoxazole, pyroquilon, pyroxychlor, pyroxyfur, quinacetol, quinazamid, quinconazole, quinofumelin, quinomethionate, quinoxyfen, quintozene, rabenzazole, Reynoutria sachalinensis, salicylanilide, sec-butylamine, sedaxane, sesame oil, silthiofam, silver, simeconazole, SJC17, sodium azide, sodium bicarbonate, sodium hypochlorite, sodium orthophenylphenoxide, sodium pentachlorophenoxide, sodium polysulfide, spiroxamine, Streptomyces griseovirides strain K61, Streptomyces lydicus strain WYEC108, streptomycin, sulfur, sulfuryl fluoride, sultropen, tea tree oil, tebuconazole, tebufloquin, tecloftalam, tecnazene, tecoram, tetraconazole, thiabendazole, thiadifluor, thicyofen, thifluzamide, thiochlorfenphim, thiophanate, thiophanate-methyl, thioquinox, thiram, THQ25, tiadinil, tioxymid, tolclofos-methyl, tolfenpyrad, tolprocarb, tolylfluanid, tolylmercury acetate, triadimefon, triadimenol, triamiphos, triarimol, triazbutil, triazoxide, trichlamide, Trichoderma afroharzianum (formerly harzianum) strain T-22, Trichoderma asperellum (formerly harzianum) strain ICC012, strain T25, strain TV1, Trichoderma asperellum strain T34, Trichoderma atrobrunneum (formerly harzianum) strain ITEM908, Trichoderma atroviride (formerly harzianum) strain IMI206040, strain T11, Trichoderma atroviride strain I-1237, Trichoderma atroviride strain LU132, Trichoderma atroviride strain SC1, Trichoderma gamsii (formerly viride) strain ICC080, triclopyricarb, tricyclazole, tridemorph, trifloxystrobin, triflumizole, triforine, triticonazole, UCQ09, Ulocladium oudemansii strain U3, uniconazole-P, urea, validamycin, valifenalate, vinclozolin, voriconazole, WLR08, zarilamid, zinc naphthenate, zinc thiazole, zineb, ziram, zoxamide, preferably azoxystrobin, trifloxystrobin, benomyl, carbendazim, thiabendazole, tebuconazole, imazalil, penconazole, procymidone, vinclozolin, mancozeb, ziram, copper oxychloride, sulfur, fludioxonil and/or iprovalicarb.

10. A kit comprising
a) the yeast strain of Papiliotrema terrestris species as defined in claim 1, or a composition thereof, and
b) one or more chemical compounds and/or one or more biological agents or compositions including them, selected from fungicides, insecticides, fertilizers, biostimulants, macronutrients, micronutrients, pathogen-resistance inducers, plant growth regulators, foliar nutrients, antibiotics, herbicides, acaricides, food additives, antioxidants, microorganisms.

11. The kit according to claim 10, wherein said fungicides are selected from 2-methoxyethylmercury chloride, 2-phenylphenol, 3-ethoxypropyl mercury bromide, 8-hydroxyquinoline sulfate, 8-phenylmercurioxyquinoline, acibenzolar, acibenzolar-S-methyl, acylamino acid fungicides, acypetacs, Adavelt, Agrobacterium radiobacter K84, aldimorph, allyl alcohol, ametoctradin, aminopyrifen, amisulbrom, ampropylfos, anilazine, aureofungin, azaconazole, azithiram, azoxystrobin, Bacillus amyloliquefaciens (formerly subtilis) strain QST713, Bacillus amyloliquefaciens strain AH2, strain IT-45, strain FZB24, strain MBI600, strain D747, Bacillus mycoides isolate J, Bacillus nakamurai strain F727, Bacillus pumilus strain QST 2808, Bacillus subtilis var. amyloliquefaciens strain FZB24, Bacillus subtilis strain AFS032321, strain GB03, strain IAB/BS03, barium polysulfide, benalaxyl-M (=kiralaxyl), benodanil, benomyl, benquinox, bentaluron, benthiavalicarb, Benthiavalicarb-isopropyl, benzalkonium chloride, benzamacril, benzamide fungicides, benzamorf, benzohydroxamic acid, benzovindiflupyr, bethoxazin, binapacryl, biphenyl, bitertanol, bithionol, bixafen, blasticidin-S, Bordeaux mixture, boric acid, boscalid, bromuconazole, bupirimate, Burgundy mixture, buthiobate, calcium polysulfide, captafol, captan, carbamorph, carbendazim, carboxin, carpropamid, carvone, cell walls of Saccharomyces cerevisiae strain LAS117, Cheshunt mixture, chinomethionat, chlobenthiazone, chloraniformethan, chloranil, chlorfenazole, chlorodinitronaphthalene, chloroneb, chloropicrin, Chlorothalonil, chlorquinox, chlozolinate, ciclopirox, climbazole, Conostachys rosea strain CR-7, Coniothyrium minitans, copper hydroxide, copper naphthenate, copper oleate, copper oxychloride, copper soap, copper sulfate, basic, copper zinc chromate, copper(II) acetate, copper(II) carbonate, basic, copper(II) sulfate, coumoxystrobin, cufraneb, cuprobam, cuprous oxide, cyazofamid, cyclafuramid, cyclobutrifluram, cycloheximide, cyflufenamid, cymoxanil, cypendazole, cyproconazole, cyprodinil, Cyprofuram, dazomet, DBCP, debacarb, decafentin, dehydroacetic acid, dichlobentiazox, dichlofluanid, dichlone, dichlorophen, dichlorophenyl, dichlozoline, diclobutrazol, diclocymet, diclomezine, dicloran, diethofencarb, diethyl pyrocarbonate, difenoconazole, diflumetorim, dimethachlone, dimethirimol, dimethomorph, dimoxystrobin, diniconazole, diniconazole-M, dinobuton, dinocap, dinocap-4, dinocap-6, dinocton, dinopenton, dinosulfon, dinoterbon, diphenylamine, Dipymetitrone, dipyrithione, disulfiram, ditalimfos, dithianon, DNOC, dodemorph, dodicin, dodine, donatodine, drazoxolon, edifenphos, enestrobin, enestroburin, enoxastrobin, epoxiconazole, etaconazole, etem, ethaboxam, ethirimol, ethoxyquin, ethylene oxide, ethylmercury 2,3-dihydroxypropyl mercaptide, ethylmercury acetate, ethylmercury bromide, ethylmercury chloride, ethylmercury phosphate, etridiazole, extract from Melaleuca alternifolia (tea tree), extract from Reynoutria sachalinensis (giant knotweed), extract from the cotyledons of lupine plantlets (“BLAD”), extract of Swinglea glutinosa, F500, famoxadone, fenamidone, fenaminosulf, fenaminstrobin, fenamistrobin, fenapanil, fenarimol, fenazaquin, fenbuconazole, fenfuram, fenhexamid, fenitropan, fenoxanil, fenpiclonil, fenpicoxamid, fenpropidin, fenpropimorph, fenpyrazamine, fentin, fentin acetate, fentin chloride, fentin hydroxide, ferbam, ferimzone, Florylpicoxamid, fluazaindolizine, fluazinam, fludioxonil, flufenoxystrobin, fluindapyr, flumorph, fluopicolide, fluopimomide, fluopyram, fluoroimide, fluotrimazole, fluoxapiprolin, fluoxastrobin, fluquinconazole, flurozolamide, flusilazole, flusulfamide, Flutianil, flutolanil, flutriafol, fluxapyroxad, folpet, fosetyl, fosetyl-Al, fthalide (phthalide), fuberidazole, furalaxyl, furametpyr, furcarbanil, furfural, furmecyclox, furophanate, Gliocladium catenulatum J1446, Gliocladium virens GL-21, glyodin, griseofulvin, guazatine, halacrinate, hexachlorobenzene, hexachlorophene, hexaconazole, hexylthiofos, hymexazol, imazalil, imibenconazole, iminoctadine, Inatreq (fenpicoxamid), inorganic oils, inpyrfluxam, iodocarb, ipconazole, Ipflufenoquin, iprobenfos, iprodione, iprovalicarb, isofetamid, isoflucypram, isopropanol azole, isoprothiolane, isopyrazam, isotianil, isovaledione, Jun Si Qi, kasugamycin, kresoxim-methyl, laminarin, Lime sulfur (lime sulphur), mancopper, mancozeb, mandestrobin, mandipropamid, maneb, mebenil, mecarbinzid, mefenoxam, mefentrifluconazole, mepanipyrim, mepronil, meptyldinocap, metalaxyl, metalaxyl-M (=mefenoxam), metam, metazoxolon, metconazole, methasulfocarb, methfuroxam, methyl bromide, methyl isothiocyanate, methylmercury benzoate, methylmercury dicyandiamide, methylmercury pentachlorophenoxide, metiram, metominostrobin, metrafenone, metsulfovax, metyltetraprole, milneb, myclobutanil, myclozolin, N-(ethylmercury)-p-toluenesulfonanilide, nabam, naftifine, natamycin, neem oil, nitrothal-isopropyl, nuarimol, octhilinone, ofurace, organic oils, orthophenyl phenol, orysastrobin, oxadixyl, oxathiapiprolin, oxazosulfyl, oxine copper, oxolinic acid, oxpoconazole, oxycarboxin, oxytetracycline, PCNB, pefurazoate, penconazole, pencycuron, penflufen, pentachlorophenol, penthiopyrad, phenamacril, phenylmercuriurea, phenylmercury acetate, phenylmercury chloride, phenylmercury nitrate, phosdiphen, Phosphite, phosphorous acid and salts, phosphorus acid, phthalide, picarbutrazox, picolinamides, picoxystrobin, piperalin, plant oils (mixutures): eugenol, geraniol, thymol, polycarbamate, polyoxin-D, potassium azide, potassium bicarbonate, potassium polysulfide, potassium thiocyanate, probenazole, prochloraz, procymidone, propamocarb, propiconazole, propineb, proquinazid, prothiocarb, prothioconazole, Pseudomonas chlororaphis strain AFS009, Pseudomonas syringae ESC-10, pydiflumetofen, pyracarbolid, pyraclostrobin, pyrametostrobin, pyraoxystrobin, pyrapropoyne, pyraziflumid, pyrazophos, pyribencarb, pyributicarb, pyridachlometyl, pyridinitril, pyrifenox, pyrimethanil, pyrimorph, pyriofenone, pyrisoxazole, pyroquilon, pyroxychlor, pyroxyfur, quinacetol, quinazamid, quinconazole, quinofumelin, quinomethionate, quinoxyfen, quintozene, rabenzazole, Reynoutria sachalinensis, salicylanilide, sec-butylamine, sedaxane, sesame oil, silthiofam, silver, simeconazole, SJC17, sodium azide, sodium bicarbonate, sodium hypochlorite, sodium orthophenylphenoxide, sodium pentachlorophenoxide, sodium polysulfide, spiroxamine, Streptomyces griseovirides strain K61, Streptomyces lydicus strain WYEC108, streptomycin, sulfur, sulfuryl fluoride, sultropen, tea tree oil, tebuconazole, tebufloquin, tecloftalam, tecnazene, tecoram, tetraconazole, thiabendazole, thiadifluor, thicyofen, thifluzamide, thiochlorfenphim, thiophanate, thiophanate-methyl, thioquinox, thiram, THQ25, tiadinil, tioxymid, tolclofos-methyl, tolfenpyrad, tolprocarb, tolylfluanid, tolylmercury acetate, triadimefon, triadimenol, triamiphos, triarimol, triazbutil, triazoxide, trichlamide, Trichoderma afroharzianum (formerly harzianum) strain T-22, Trichoderma asperellum (formerly harzianum) strain ICC012, strain T25, strain TV1, Trichoderma asperellum strain T34, Trichoderma atrobrunneum (formerly harzianum) strain ITEM908, Trichoderma atroviride (formerly harzianum) strain IMI206040, strain T11, Trichoderma atroviride strain I-1237, Trichoderma atroviride strain LU132, Trichoderma atroviride strain SC1, Trichoderma gamsii (formerly viride) strain ICC080, triclopyricarb, tricyclazole, tridemorph, trifloxystrobin, triflumizole, triforine, triticonazole, UCQ09, Ulocladium oudemansii strain U3, uniconazole-P, urea, validamycin, valifenalate, vinclozolin, voriconazole, WLR08, zarilamid, zinc naphthenate, zinc thiazole, zineb, ziram, zoxamide, preferably azoxystrobin, trifloxystrobin, benomyl, carbendazim, thiabendazole, tebuconazole, imazalil, penconazole, procymidone, vinclozolin, mancozeb, ziram, copper oxychloride, sulfur, fludioxonil and/or iprovalicarb.

12. A method of controlling a phytopathogen by using a yeast strain belonging to the yeast species Papiliotrema terrestris as defined in claim 1, or a composition or a kit thereof, against the phytopathogen of one or more plants or crops, before sowing, pre-harvest or post-harvest.

13. The method of controlling according to claim 12, wherein said one or more plants or crops are selected from cereals, such as wheat, barley, rye, oats, rice, corn, sorghum; fruit trees, such as olive, apple, pear trees, apricot, nashi, plum, peach, almond, cherry, persimmon, banana, grape, strawberry, raspberry, blackberry; citrus fruits, such as oranges, lemons, mandarins, clementines, grapefruits; legumes, such as beans, peas, lentils, soy; horticultural crops, such as spinach, lettuce, asparagus, artichokes, cabbage, carrots, onions, garlic, tomatoes, potatoes, aubergines, peppers, fennel; cucurbits, such as squashes, zucchini, watermelons, melons; oil plants, such as soy, sunflower, canola, peanut, castor, coconut; tobacco; coffee; you; cocoa; sugar beet; sugar cane; cotton.

14. The method of controlling according to claim 12, wherein said phytopathogens are fungi, such as for example Basidiomycetes, Ascomycetes, Deuteromycetes, Oomycetes, fungi with sexual or asexual reproduction, fungi with biotrophic or necrotrophic activity, protists or Chromista.

15. The method of controlling according to claim 14, wherein said fungi are selected from the Albugo spp.; Alternaria spp.; Anthracnose; Armillaria spp.; Ascochyta spp.; Aspergillus spp.; Blumeria graminis; Botrytis cinerea; Botrytis spp.; Bremia lactucae; Cercospora kikuchii; Cercospora sojina; Cercospora spp.; Cercosporella herpotrichoides; Cladosporium spp.; Claviceps purpurea; Colletotrichum spp.; Corynespora cassiicola; Diaporthe spp.; Erysiphe spp.; Fusarium graminearum; Fusarium oxysporum; Fusarium spp.; Helminthosporium spp.; Leveillula Taurica; Macrophomina phaseolin; Magnaporthe oryzae; Magnaporthe spp.; Melampsora lini; Monilinia spp.; Mucor spp.; Mycosphaerella graminicola; Mycosphaerella spp.; Oidium spp; Penicillium spp.; Downy mildew manshurica; Downy mildew spp.; Phaeosphaeria spp.; Phakopsora pachyrhizi; Phakopsora spp; Phoma spp.; Phytophthora spp.; Plasmopara viticola; Podosphaera spp.; Pseudopernospoea cubensis; Puccinia spp.; Pyrenochaeta lycopersici; Pyrenophora spp.; Pyricularia oryzae; Pythium spp.; Ramularia spp; Rhizoctonia solani; Rhizoctonia spp.; Rhizopus spp.; Rhynchosporium spp.; Sclerotinia sclerotiorum; Sclerotinia spp.; Sclerotium cepivorum; Sclerotium rolfsii; Sclerotium spp.; Septoria glycines; Septoria spp.; Sphaerotheca spp.; Stemphylium spp.; Stenocarpella maydis; Thielaviopsis basicola; Thielaviopsis spp.; Tilletia spp.; Uncinula spp.; Uromyces spp.; Ustilago maydis; Ustilago spp.; Venturia spp.; Verticillium spp.

16. The method of controlling according to claim 12, wherein said phytopathogens are bacteria, viruses or nematodes.

17. The method of controlling according to claim 16, wherein the bacteria are selected from the species Pseudomonas spp., Xanthomonas spp., Clavibacter spp., Ralstonia spp., Erwinia spp.

18. The method of controlling according to claim 16, wherein the nematodes are selected from the species Meloidogyne spp., Heterodera spp., Globodera spp., Belonolaimus spp., Pratylenchus spp., Rotylenchulus spp., Trichodorus spp., Paratylenchus spp.

19. The method of controlling according to claim 12, wherein said yeast strain or a composition or kit thereof are applied on one or more parts, intact or wounded, of the plant selected from seeds, aerial parts, leaves, stems, trunks, buds, gems, branches, stems, flowers, fruits, roots.

20. The method of controlling according to claim 12, wherein the quantity of live cells of said yeast strain distributed per unit of culture surface ranges from 10⁸ to 10¹⁵ CFU per hectare.

21. The method of controlling according to claim 12, wherein said yeast strain or said composition or said kit thereof are applied to a solid growth substrate, such as soil, preferably at a concentration of live cells of yeast strain ranging from 10² to 10¹² CFU per gram of solid growth substrate.

22. The method of controlling according to claim 12, wherein said phytopathogen is selected between Botrytis cinerea and Plasmopara viticola of vine; Botrytis cinerea of strawberry, Botrytis cinerea and Phytopthora infestans of tomato, Venturia inequalis and Stemphylium spp. of apple and/or pear tree, Monilia spp. of stone fruit and/or Phytophtora spp of potato.

23. The method of controlling according to claim 12, wherein the kit comprises a) the yeast strain of Papiliotrema terrestris or a composition thereof, and b) one or more chemical compounds and/or one or more biological agents or compositions including them, selected from fungicides, insecticides, fertilizers, biostimulants, macronutrients, micronutrients, pathogen-resistance inducers, plant growth regulators, foliar nutrients, antibiotics, herbicides, acaricides, food additives, antioxidants, microorganisms; and, said a) and b) are used separately or sequentially.

24. The method of controlling according to claim 12, wherein the yeast strain belonging to the yeast species Papiliotrema terrestris is used as a plant protection agent.

25. A method for the control of phytopathogens in agricultural crops comprising or consisting in applying the yeast strain belonging to Papiliotrema terrestris species as defined in claim 1, a composition or a kit thereof on said crops or on the soil in which the crop grows, before sowing, pre-harvest or post-harvest.

26. The method, according to claim 25, characterized in that said application takes place on one or more parts, intact or wounded, of the plant selected from seeds, aerial parts, leaves, stems, trunks, buds, shoots, branches, stems, flowers, fruits, roots.

27. The method according to claim 24, wherein the dose of said composition ranges from 10⁸ CFU to 10¹⁵ CFU per hectare.


SEQUENCE LISTING (4 sequences)

SEQ ID NO: 1 - DNA, 525 bp, Papiliotrema terrestris
ccgggaagagctcaaatttgaaatctggcgtgctcagtgcgtccgagttgtaatctatagaggcgttttccgtgccggactgtgtctaagtcccttggaacagggtatcaaagagggtgataatcccgcacttgacacaatgaccggtgctctgtgatacgtcttctacgagtcgagttgtttgggaatgcagctcaaaatgggtggtgagttccatctaaagctaaatattggcgagagaccgatagcgaacaagtaccgtgagggaaagatgaaaagcactttggaaagagagttaaacagtacgtgaaattgttgaaagggaaacgattgaagtcagtcgtgactgagaggctcagccggttctgccggtgtattccctcagtcgggtcaacatcagttttgttcggtggataagggcagctggaaggtggcacctccgggtgtgttatagccagctgtcgcatacatcgaatgagactgaggaatgcagctcgcctttatggcggggtcgcccacgtcgag

SEQ ID NO: 2 - DNA, 35291 bp, Papiliotrema terrestris
  misc_feature (243)..(252): n is a, c, g, or t
  misc_feature (1457)..(1466): n is a, c, g, or t
  misc_feature (5249)..(5258): n is a, c, g, or t
  misc_feature (8813)..(8822): n is a, c, g, or t
  misc_feature (11138)..(11147): n is a, c, g, or t
  misc_feature (12118)..(12127): n is a, c, g, or t
  misc_feature (15728)..(15737): n is a, c, g, or t
  misc_feature (16417)..(16426): n is a, c, g, or t
  misc_feature (17927)..(17936): n is a, c, g, or t
  misc_feature (20030)..(20039): n is a, c, g, or t
  misc_feature (20689)..(20698): n is a, c, g, or t
  misc_feature (27154)..(27163): n is a, c, g, or t
  misc_feature (29773)..(29782): n is a, c, g, or t
  misc_feature (30460)..(30469): n is a, c, g, or t
  misc_feature (34959)..(34968): n is a, c, g, or t
ctatgagtatagcactaggaagctaaccattagggcgaatagccctgtagcttcggctagagcgaatcctagaatagcgtatgtgaatagttgtcctcgaacagcagggtttcgagctgtaccagcgattagagcgctgaatactgtaccgatcccaacaccggctcctgagagaccgatagcggctagaccagcaccaatgaactttgcagcagccattgtgtattgtgattgtctgaaccnnnnnnnnnntcaatgaattgagctttagcctttataatctaaaggctagtgatgacatccatgaacggctatcacctgtataaagacagtgatagaatatatgtgatcatggaggggacagacgtggatctctattatgaactctatagtgcggtagctggatctcagactatattaccgtatctacccttttaggacctataatcatgcttataatatctatagatggtagattacctaacactcggtgctatagtaggttataggagtattatactcctataaccataaactatgaatggctactatcagcatgagcaatgactctatttaccgctatagtaatggtgtactctaccatttttcctataggatcctacctatccctataagctatagggttttctatgaaaatgctctacaatacatgtatgctaacgtgagtcaatgagtatagcctcggaaactttatttatgcacttctgacgccatgctgaatataatagcgccacatgtatgcatagcttttatctctctctatacaagttattatttattagtaaatttattactaaatttatttttaataaataaattattattattattatattggtatggacggacagagcaagattcgaacttgcggtgggcgggacccactttgaggttcaagctcaatgccttagaccactaggctatctgtcctgttatctcatcgactcacctgctacccttaaagaggggtaggaaaaatggtgaaatatagtatattgtctatctataaacttttataagaatagttacggtcatagatttttatcgtctcgtctctacacaatggctataaccccttactattaaggccagcctatattttttgctgctataggtgacacagaatatttatgttaaagaacataagggggctagtataaagtccaagcataatagctttgtaacgtatacacggctcacaatcagttgaaggatcggaggtagtacgaacactgatacaacgtataccagtgcagccagagtaaggagaacgaatgctccttggttaaggaagtaaaatgggagagcttgaggcataggctgatatgcaacaggcaatgtgataggtgtttccatcatcactatgaatactataattatgagtgactatatagcatgctcataataacaatagactcttacatactttcctttcnnnnnnnnnncttctgacgccatgctgaatataatagcgccacatgtatgcatagcttttatctctctctatacaagttattatttattagtaaatttattactaaatttatttttaataaataaattattattattattatattggtatggacggacagagcaagattcgaacttgcggtgggcgggacccactttgaggttcaagctcaatgccttagaccactaggctatctgtcctgttatctcatcgactcacctgctacccttaaagaggggtaggaaaaatggtgaaatatagtatattgtctatctataaacttttataagaatagttacggtcatagatttttatcgtctcgtctctacacaatggctataaccccttactattaaggccagcctatatttttgctgctataggtgacacagaatatttatgttaaagaacataagggggctagtataaagtccaagcataatagctttgtaacgtatacacggctcacaatcagttgaaggatcggaggtagtacgaacactgatacaacgtataccagtgcagccagagtaaggagaacgaatgctccttggttaaggaagtaaaatgggagagcttgaggcataggctgatatgcaacaggcaatgtgataggtgtttccatcatcactatgaatactataattatgagtgactatatagcatgctcataataacaatagactcttacatactttcctttctatgagaggttggttagttagcaagggcagaaggggactatagttattggtaaccatataaggtcgttataacgtttcaacaagcctatgattggataggtagcatgtcgaatgcgtggtgtggagtaggtgatggtagtacccactcgattgtgtaggcgaaccgtgtgcttcgttcgtacgttaggttgtccgtgaagtatagcggttctccccatgggttgtcaccaacgtacttgtcgttagtgaacatgtcgtaaacagtgtacaggaacagaagagtagcagagacgctaacgaatgaaccccatgatgagattaggttccatcccgcgtaagcgtcagggtagtcagcgaatcgtcgtggcattccttgtagtcctaggaagtgttgtggcataaatgtcatgttaacaccgatgaagaatgtccagaagtgcgcttgagctaggtgttcttcgtacatcttaccgaacatctttggagcccagtagtagaacccggcaaagatactgaatacggcacccattgaaagcacgtagtggaagtgagccacaacgtagtatgtgtcatgcatcgctacgtcaacgctcgcgttcgctagaactacacctgtcagtccaccaatagtgaacagaccgacgaatcctagagcgaatagcataggtgctagcagtcggatagaaccaccgtaagcagtcgctagccaactgaagaccttgatacctgtaggtaccgcaataatcatagatgcagctgtgaagtaagctcgtgtgtctacgtccatccctacggcgtacatgtggtggctccatacaatgaatccaagcacaccgatagaggcaatggcgtacaccataccaaggtaaccgaaaacaggctttccactgaatgttgaaacaacgtggctaaccataccaaatccagggataatcattaggtatacttcagggtgtccgaagaaccagaataggtgttggtataggacagggtcccctcctccagcagggtcgaagaatgatgtgttgaagttacggtcagtaaggatcatagtaagagctccggctagaactggaatacatagaataataataactgactgtgatagcatcgcccatacgaatagaggtgccttgtgtaggcgtagccctggagctcgcatgttaaggattgtagtgataatgttaatggcaccaagtagagatgaaatccctgagaggtgcaggctgaatacagctagatcaactgatccaccactgtgtgattgtacacctgttagaggcatgtacatagtccaaccgctacccataccttgttccacgaagacgcttgaaaggataagaacgattgcaggaggtagtagccagaagctgatgttgttcagccgtgggaacgccatgtccggcgcaccgataagaacgggagccatgtagttagcgaacccagccatagcaggtaccaccatgaagtaaatcatcatgataccgtgagttgtagcaattacgttgtagaggtggtggttaccagaaaggaactggttaccaggagctgatagctccatccggataagtacactgaacgcagtacccagaagaccggtaaagacagcaaaaaggatataaagggtaccaatatccttggcgtttgtagagaagagccatcggctagccatgttaataagagggttatgctgttatggggaggggtaggtaggttgacagctgctggtgagagtaactatagtgcatagcaatattcatggtagagtattctatcactataggctctagttatctaccataaaaatggacatagcggatagccatgagaactaaaacagtagaaactgatagatgccttaataggcagaaagtgtcacaaatgtgaggtctttaatccacaactaagacttagagtaaatagaagggaggaggttcacggtattagtaaatcatagaggagagagagagatgcatcactagtaacccatagggtacacagcagtgctagtagcaagtctttaataatcatatgtgatagatggcggaatagcctaatctacacactattagatgtaggtactttagatatgatataatttaaaacatttatagctgtaattataaaaactcgctataggcttataatattataatatgaccatatattttatataaagcataacaactatgtatggcaatataagtcctatagccatctagcactacaatgcagagtgtctaggtatgacagtttaagtaacagcctatatacataacaactaaaccttataactgtacagggtcttagtccaccacatgtcacacacgagtatggacttctgcttaggcaaagcccctttatactaaccttcccatcagaaacctttatactcaaccaaagggttaatgttaggtttatagggtagttgttatttaaaaaataaggaggggttatggaggcatgctttgcgagtataccataaaaacaaataacatataaatatgactgcacaacaacgatcatttgtcgaggcgctactacgtctacggccgctattccaatcacaccctcaccaccttactctggaatcaccatggccaatccttacaagtggagctgtgctagctatgctttcaagcgctgccctttggttcaacggtctacaatactcaggtacattccttatccttgggatgcttagcacactaacagctatgatcctatggtgggctgactgtgttaaggaaggtacataccttggacaccacactaaggtagttcaacaaaaccttgctatgggtgttgcgcttttcattgtaacagaagccgcactatttgtgagcatcttctgggcctacttccactcatcactagcacctacagtggaactaggtactcaatggccacccgctggtatcacagccctatcacctatggctatccccctactcaataccgttctactacttagcagcggtgctacagttacatggggtcaccacgcattcttctgccgaaaccgatcagctgctcttcaaggacttatnnnnnnnnnncaaggacttatgtttacagtagctctagctattatcttcactgccctacaaggtgttgaatacaacatggctggatttactattgctgacggtgcttacggtagctgtttctatatggccacaggtgctcacggactacacgtgctggtaggtacactagctatcactgttggtcttgcacgacttattaactaccaacttacatcaactcaccacattggtctagaaggagcaatcctatactggcactttgtggacgtcgtttgggtcatcctatacgttacagtttactggtggggagctccattatccctatctaaattagtcccctatttatatattttccccttccagaatactacttaccttgttatacttaaccttttaattataagttaattggctgagtagaatagggttacaatagaggtagatgaggaacaagaacactatagtatgcttagttaccatagaaaatttaaatttttataagctatgaagtgactatttcttaaatggctatcagttatttaagacaacttgttagaagtcaatagtcataatatctaatagcatggctcatttttatattaaaatattaacagtatttaaaatttaaagtcatttaatggtatactgcgtttagtataaaaatagaggattatattgacacgtttaccagccgaattaatacaattactaaatttagagtgagtctaattcaggtgggctgggctcgaaccagcggcctcatgctcccaaagcatacgtcttaaccaaactcgactaccacctgtgtactcttagctaccggccccatacaaaccatcattaaagttgataattattgatactattaacctattgtagtatctttaatgaggggtctaaactggtacaacgtcaaacgcaacaagtacgcttggtaccagtagaagtagagcgatagctggtggtagcattccagtccaacagaaagaaagtagttgatcgtaccgaagtcgtggtagtgtagcccggaaccacacaaaaccaaaacaaccggcacaagtcttaagcgccagcactagtgatgatagtgacagtggtgtaccattggcaataacttcagggaatccccatccacctaggaatagtgtagctgatagcgtgctcatgagaacgatagaacagtattctcctaggaagaagagtacgaaaggcatagcactgtgctctgtcatgaacccagcaacaagttctgattcagcttcaggaaggtcaaagggtgttcggtttgtttcggctagtacagatacaatgaatagcatgaatagaggcgctagaggaacacagtaccagataggtgcttgtgattctacaatagccgtgtggttaaggctaccggccatgatcaggactgctagaacagcagaacctaggatcagttcatagctaatcatttgagctgttgatcgtagcccaccaagaagagcgtatgattcgttggcagaccatccagctagtagtaccccataaacaccaacactagaaacagctagagagtatagaacccctagttctaggtcagcaatagcggcaccaggaccgaatggcataacagcccaaccaagaagggcgaacaccagtgtaataacaggtgctaggtagaagatgacggtgttagaggcccccggaacaacagtttcctttaccaccagcttcagggcgtcagcgaacggttgtagtactccatagacacctactgcgttaggccctactcgtcgttgcattgcagccagaaccttacgttcgataatggtcatgaacgcaacagctagaagaataggtaccagtacagctagcacagacagaatggagaagagagtagaaagcatattacgtagtttgtagacataaaacccgtgatacttaaaagactaagaccagtcgggaggtatgggagaggtacactataaataataataatgaatgaatatatatagacagacagtacataactgtgccgtatggttatatttatcaatcttacctaattgttaaaacatggttataataaccacccttataagtataactaaggtagtctataatggttataggaaccagaatagtcatgaatataagagcacaatggctcctagcgatacgttaagataaatagtgtggtgatagttaaataaaggggtctatagcaatggcagaaataaccatagtcaataagacctaggcttgtgactttagccatgctaggtattctggcaggctaaccgcttcaatacaaataggcatgaaggcgtggtaagctccacaaagttcgctacattgtccgtagtaaacaccttgtcgttggatcagagtagatacttggtttagtcgacctggacaagcatcgaccttgatacctagtgaaggaatagcccagtcatgcagaacgtcagtagctgttacgatggctcggatgtgcgtgtcaactggaagaactagccggttgtccacttctagtagtcgtagttgcccagcttctagttcgtcagcaggaaccatgtatgaatcaaatgcaactgtttcgttgtcgtcaccagcaaagtctgatagttcgtatgaccagtaccattggtgtccaataaccttaacagtcatagctgggttaaccacctccatgtggttccccttgtaaacctatagaaaaggaatagggttgattatggactatgccttgcttcccaccacgatagttcataactatgttaacaatcctgaaaggacttaccccgaccctcctaacacctagtcttttttattaccctacccccccaagggggggttaaaaggtgggttagggcgtaaggagggaagtagggggttaatgtgggattatagcaccgttgtagtctctgaggcctacggcctacctgtacgggatataacccagccaggtatacagtggtaaatgcatggacgagagtaactgtgttgatttttccaatttttttacctatactgagcctagtataggggaggtttaattaaaattatcaaacacagctcatagctatgatttctatgtcctatagggagtccatgaggtatagcagcttgaatgaagggaaggcaatagccacaaggataagtgctggcacaacagtccatacaacttcaataagagtaccgtggttgttgtaccggtgagcgataggcgcttgtgtgtccttgaaagagataacgatagatccgagcatccatcctactgccacaccaataacgatgaggtagaagaagatagtgtcgtgtagttcagtgatcccctcgaatgatggagaagcaccgtcttggaatcctagaagccaaggttctggagcgtcacaagtagctgtgaatagtgatgtaaatagtgaaagcatgctatataatgaatattaaatataaactagtcgtgtcaatcgctaccaatgataaaacactgatagtggtactagaccaatgtgacgtcatagtcactttattaatcgcataatacaaccttgttgagaggtgaaaatgagataataggtnnnnnnnnnngcggacgtgtcaggtagtaaggggtactgataaccataaatggttactagctaataacggttagctgaatcgaacagcctttactaatgagatcaacccattctttagtaatccaataccgtgtatattaataaaagtcagtctaaataaaagatattgtattgttaatagacaactctattataactatttggtaggtaagaagagaaatatattacacaccatagctataggtctctctaagtacaatgagagtggtaaataacctatatctattaggggaatattagtccttatgacctaacagccccaacccaataactaatgggtatgtaagaaggttaataagaggttggtagaatttctctattgctactagtgtataggcctatacttataactataggtctctacttatatatatatatcagttaggtgtacttataacctattactagtagtgtattattaataccaaaaggtgagtagggggctaatttaccattcatctatcagccgcaggttcccctacggctaccatggttcgacttcagaccagtcagctggccaggctctgtagcaagctccgccattattactgtcttccagcacccattgatatcaatgggaggtctctccatcattatgactataggttgatgtataattataatgtggatatatatgagactatagttaaatacaagcagcgctcacccacgttaccctaccagcatactagcccgtgacgagcggttagtaccccagagagattaaacttcaccgctccatacttatgagcgattactcgggactccagcttcacgcgaacgagtatcagctcgcgatctgtatgcttgtggtagttttggatgctttcccattttataaggctagctctcatctgacacacacacttgtggcacgcctgtagcccttcccataagggccatgacggcctgacataggcccgtactcgtatcccttataaggctactactgcctatacactaatcaaattaattagcatcatagacagacgggtctgcgttcgttcattggactcaaccgtagggctcacgaccacgaactgacgacggccatgcaacgcctgtaaattaccagactcgctctaaattaataaactattaaataatgagcgagccaacagtaaaatatgcaagggaaggtaaggttttaagcgtatcgtctgattaaacagcatgccccgcgcccgtgtgctctccggcggtctcatcgttagtgtgtcggtcttgcgactataccctacaggcgagttgctcaggcgttagccttccaggggttaaaattgtgactatgcttgacgtcaagtattacactctatattcaagtcgcactccccactgcggcaactatggttcactgctggggctaaacaggctctactctgtgtcggtatcccagccttcgtaccttaccgtcagtctgtcaccagcaccaattgcattgcccacccctagggatcataggatctcagccctccccccagtgttggtggtaccaaatgtagactcgtatcctattagagatggctgcgtaccctatacgcccattcaacaccataagagctgacccgtctgtgttaccgcgactgctggcacagatggttggacgggtcttgtacataataagcatcaatatgacttattatatctggatattaagatatccaatctaggtcacctggtcacaccttttccctaacccccctacggggggttagggacgcggtgcattgccaaggattcttcactgctgcgcacatgggtgctctggctggccagtgtcggatgcatagctatcactatccccttcccgtcatagcctaagtaacctcatagctggtaggggctgaatctacgacaatatggtggattacctttgcaccatgtataagcattaactgatatcacacaaactcttaaaaaaataaaagtagtgatcaatttagaatactattcatgatgattatccctgcgctataggggccattgcgtatgattccagcactgctgaccatttggctcctattcaccataatgaatcggaaaacttgcatgtgttatacgcctagatacccttcatggctcgccacgatgaaacgatcgcgattatcccttctatcaatacctatattattacttattatcatcaatataggtactacgccccactaaccccttactaacccttacccctccaatggagaggttaaaaggtgggtaagggcttgggcggaaagggtctagatcatcccagttaccacattcaatnnnnnnnnnnattgtgactatgcttgacgtcaagtattacactctatattcaagtcgcactccccactgcggcaactatggttcactgctggggctaaacaggctctactctgtgtcggtatcccagccttcgtaccttaccgtcagtctgtcaccagcaccaattgcattgcccacccctagggatcataggatctcagccctccccccagtgttggtggtaccaaatgtagactcgtatcctattagagatggctgcgtaccctatacgcccattcaacaccataagagctgacccgtctgtgttaccgcgactgctggcacagatggttggacgggtcttgtacataataagcatcaatatgacttattatatctggatattaagatatccaatctaggtcacctggtcacaccttttccctaacccccctacggggggttagggacgcggtgcattgccaaggattcttcactgctgcgcacatgggtgctctggctggccagtgtcggatgcatagctatcactatccccttcccgtcatagcctaagtaacctcatagctggtaggggctgaatctacgacaatatggtggattacctttgcaccatgtataagcattaactgatatcacacaaactcttaaaaaataaaagtagtgatcaatttagaatactattcatgatgattatccctgcgctataggggccattgcgtatgattccagcactgctgaccatttggctcctattcaccataatgaatcggaaaacttgcatgtgttatacgcctagatacccttcatggctcgccacgatgaaacgatcgcgattatcccttctatcaatacctatattattacttattatcatcaatataggtactacgccccactaaccccttactaacccttacccctccaatggagaggttaaaaggtgggtaagggcttgggcggaaagggtctagatcatcccannnnnnnnnnatcatcacagttaccaccttcaataaacttattggtaggttgaaggctatagtctggatctaatgatcgtcttatgcgtaattaaacgcggttcgtgttgtactattaggtaaaaattttgtagctaaacgctctaagttgatatagctatgataagagcgatgaatttattgaccaacttctgagtcgtcaatggggacgaatactttacctaatctgtatgatacaccattcataggcatagggtaaattagaatctatattactagcaaaattacccctatcaaagtataatctttcgtactaggtaataacatggtggtactcttcactaagttttaccatttatcaactctagaagagggtgtagtaggaggggtgcgaaaccataaggaccacagtggacattattgatagttggtgggaataattagtcatcatcatcatcataacaataggccatgataaacagggtattttttgtaatcaataattataaatataactattaccagtagtgatgagtttataattatgccctataactgtctatgttttccattcctatggtgattataaggggtctatgctatgatgataggttgtgtaatgcttatcgtgatagctttgatggagcgttgtcaccaggtagtagtgaaacaggtgtaatcgctgaacgttggtctgtgatggccagaactccagaggctacttctactacgaatcctagtgtcagtactaggaagaagataacggcaacccagaaaccgtatagaccaacgtgtccaaggctaggagcaagagggatcagtgtagccacttctaggtcaaaggctaggaatagcacagcaacaaggtagaagctgatactgaatggagctcgtgtttgtccgcgagaggcgtcatagccacattcgtaagtagtgaccttttcagtgtcaggacgagctacagaaagtagtgcgtttacaagcagtagaatcacggccagtacaggcacaatgataacaaggctgtatagagtagtactcatagtcatggttatacgctaaatagtgaaagagccaggagacgagcgctatctagtagtagtgatggtgatgccatgtagaagactaggatggcagtaagtagagaaatagtgtatgattgtacagctgtaatgtctacaggcttttctgagttacgcataacagcccagtaatagtcccatcgagagttagcaaagtggtcatgagggaatgtttgtgggctgactggtgctgatgggtcaaaccaggctactcgaataacgttaaggtagtatgatgcactgataacgctagagaggatagcaactagtgatacgaagtagtacccaccactgatagcgctgtaaagcacttctagcttaccgaagaaccctagtagtggtggaacaccagctagtgagaatagtagtactagtaggctaaaggctagaggtaggtttcgtacacgtagaccagctagttgtgagatcagtgttacgtcatgggtaacgtcacctccacgagatgagctatggctaatagctcctagcaggctgaatgataggatagctgtgagtgtgtattgtactaggtagaacacaaaagcgctagtagcttcagggctgtttacactcatagctagtagcatgaaccctacgtgtgagactgtagagtatgctagtagtcgcttgatacgtcgttgtgctagaccaactacagcacctacaacaagagacagtgtgctactcataagtagtagtagtgcccatacgtcgtaagaggctgtatcaacaacaacaagcatagcactgataagaccactgcttagggtgtatagtagtgtcagtaggctgatcttaggtagaactgcaatccatgtagtcacaattgttggtaccccgtcataaacgtcaggaccccagttgtggaaaggagcagctgtaaccttaaatagaagaccagtcataacagctgtaaagccaagagcaagagcccagttgatgctgatagagtctgatccaccaacactcagaagtgtgactagagattcaaggttagtaatacctgtttggttgtaaactacggcaaaacctagtaggataagacatgatgatagaccacctagtaggaagtatagtagaccactatgagtagcagactctgaatcacggtaaagagctgctagtacatatacagcaaatgactgtagttctagagccaggtatagagtaaccatgtcaccgctagcgacaaggagacatccacccatgctagaaagaagtgcaaatagaggataagtggcaatacgaggagtagccataggtgatagatgagcgtgaggagcccattggactagagcaatagcacctacagaaagtaggaatacttgtgcagctagtgaaagaccagtcagatcaataagtccactgtatagaccaagacctgatcctaccccctcccagctataggcattggcggccagtgcagcagctgaaaggaatgtgaggactgtaactcgtgtgagtagagctggcgtagcatagaccgaaggaagcgcaacggctgtcagtagaataaatagagctagtgtaagcattactgttggcggtaataataatagttgttatctcaataatcataaagagctatagtactatacctactccctattatagaccctattcatgcctaataactataatatccaagaaatctttgattactaaataacaactctatcattatatatgcctatattttctgggtatattaattagacgacaatccatagatatgaataatacatatggcttatacctacctataggctttaagaattagatcctaataccacccctcagtgttaggcatgccatgtctttaataacattactattctctatccctgtgaatctcgtaatacaataaaaagtatgttaagagtatgtttgttttaaatttaaatctactcactaaccctatggccataggactcaagctgtaccgttaattagcatttaactataatgatacaaattagaaatcatatcctaaggaatatctgtcgtataggttttaatacaagtaccgtgatttgcacacggaccatctgcttggaaggcagagacgctactcttacgccatacttgtcagggaaagggtcatttaatctattgtgataagtatatttaccattatcggttgggctacttcactgtactatttaaatggtgagtcttgtactattagaagaataaatcagatattcttagtaacacttcgcttgaataggattaggtatagtaaacagtggtactacttcttgttataccctctaccacccctactaacccaccaaaggtgtgttagtaggcaggttaagtgggtagagggaaggaagagaagggttagtatcggagggataatcttaatgattgtagacgtattattacttatacctcataggtggtgtatagagtttataattttgatcctttaaagaggctatataaatggagtgacacggactcgaaccgtgctctcagcgatgcaaacgcagtatactaccaactgtactatcacccctcccnnnnnnnnnncacccctcccttagcactagttatttaagttttttctattctaaggggtctatagataatatctattgtcgtgctcgtagacatagagcaataggacccagcatcgctagtagcagtacgaaactgataagaacgagccagaggaatccataggtgtaaaggctaagaccaagagcttgcacttgtgataggtatgcgtatgaggcatcagctgcttgtgaaccgtgtacaacgaatacatcagtcattgatgatgtaataactggtgctccatcaccaagaagcagagtgttaaggctgtatagtgctccagtgatagcgctgacggttgttgatgttacgctaggcattacgctaaaggcttccagtaggaatactccagcaatgatgaaaactagtggtagtccttgagtaaattcttgcccgctagcagtaatttcagagagacgtacgttaagcatcatcaccacgaataggaatagcacagcaacagctcccacgtatacgactaggtatgtgagacctacgtacgtgatacctagacatactaggtaacaagcagctaggacgaatacaccaattagatataggatagctacaatagggctgctagctgtgatcgtcattacagcgcttacaacagtggctagtgccagtagttctagtacaaagctagtcannnnnnnnnngtgacagtcatgctgatactaagcgataaagtaaaaagcatttattcactgtcatagcatccaggtcgagcttgctgccatagatgtcccactgggaatacacatcgtcaccactgtggatcgaacacaggtcggagcattaacagtgccctgctctaccactgagctatggtaaccatcgtctatcacccctcttatagacctaaactatatgcttgcttattaaaatggtaaaatactatcactagttacacacttaaccatttattaacccctctacccctttagttacactcttataccctctcccctgcctatcctcttacccccccccttggggggttaaaaggtgggttaaaaggggatagggggtcaagatgtgatcccattaattttcaatttatttaaattaatttaaactagtgaagttcaatagcgtccttcaggtatgaacatgttagaacacagaatacgtatgcttgaatcagagatacagctagttctagaccaataagagctacaaacacagcaaatggaattagtgtgataactgctacaataactcctgcgctgaatagcttagctaggaaagtagcaagaatcttcagtagtgtgtgaccagcagtcatgttactgaatagccgaacacctaggctagcagctcgtgcaaggtatgaaactgtttcgatcagtaccagcataggaaccagagctagaggagtaccgcttggtacgaagtatgagaagaagtgtactccgtgtagacgtagtcctaggatagttacagcaaggaatacaaatactgatagtccaagagcagcaataccgctagtagttactgtgtatccgtatggaatgttaccgtttaggttagctacaagaataaacatgaatagcgcgtagatgaacggtgtgtagatttcgtgacggctacctacttggtctcgaaccatacctgcaagtgttgcgtaggcagcttctagggctagactccatcgacttggtactaggctgtacccgttgttagctacaacgtgaagtgatactagaagtcctacagtaaggagtgtgtaaagacctaggttagtaagagctagaacaaaatcaccaaccacaggtacgtggaggctaatgagaggaacaacttcaaattgttcaagaggagaaaggattactgcttgtgttgtcatttattgagttttaagattaattaacagatgttaagaggtgttgacaacaattattagcagtttttaataggcataataatatattaaaccctaaaagaatttaatagttcgaaaaaggcttaataagcaagaaaaaggaggcaggtttaagacccctaataggtttggtttaatagttgttagaaaagagcttaataagtaaactgtttaaaacattaaaaggttgttattactactcttactgaagatttattaatctaggctagggagcttcctagcctcttattttcaaacaaggaaacaagaaaggnnnnnnnnnnatagtttgtaatgatgagcttgtgagcggcctcacgcgtggactgcaactctgtgcgaaggggttcaactcccctcaagctctattccttgtcttggcctagcccataactattatgatagttatgtctcatagctatttaatgaaaaaccattcaaagctattgattatagcctacttatagttattaataataaatcccctctagacccttttagcaatagggatggtaatagtgggttgtaggcgtacagtgctgaacggtatcgcacgcctttttattttttattaaaaaaaaaatcagaccgtaggaagctcagagagccatatatagtatgccttgccttataagctactcactatacaaatccttcactaacattatcattcccttactaacccatatatatatatatatatattttaattgagaatgaggggaaagtgttagtaacatgtgaggcagttattacatgataaggatagcacaagctatggcaataggcatagaaacctatggttattactatctgggatttacccataactagtgattatccatgagaagagccacccatactaggcccatgtatatagaccatatcagcatcaacaggactctggctatgaatgtctatagccccctcctccccaccatttctaacgctacaacttaatttgtgtactctcctaagcataaagataatcttattagcataaaaagtaaccaacctaatagtataggtctagtatagatatttgtatttatcactataagtatggttaatgtatcacttttattaaaatattaaataggggtctatttatccactaatagtcggtgtgtaagcataaccttgactgtatacacgttcttcttgttgatggctgtggtagaggctgtgtctagcacttgttggtcgttagccgttagtgaacctacaacgattcgttgttctgtcatacgtggtcgtgctggttcggcagatagacgaccatgaactgatacttgtacccctaccagactagctggtagttgtactccattgacactagggtaggtagtgataggggtagcattagatactgtaccaatagtaccaagcatacggttgataacccgcccaaacttcttagtcttaagttcccgtgctaggtagtgaccaagaacggtagcgtccatatatgggcctgatagtcgtgtaacgattagcattactggtcgttggcatacttcggacagtgccgttcgtagttgcataatagttagtgacgatagggcatggtttgatggtgcgtagtaaccgaccttcacagtaatacctcctacaggaagtccagctgcaggacgtgtaatgattgtaggtcgtccccaggcgacttgatgtccagcctcaaaatggttgaatacctttagggtattgttgatggtgtgtagtacacgttcgtctacgtaagagtctgatacgtatgtgtgtagtgcacctggtgctagttgtagatgtgccatattaataatgatgaaaaaagtgtgcgaccaatggataatgatcataaggatgtattgagatggaacagttaattgctatcgagcatatattatcattcatactgattcccgagaggaaggattgaacttccgtattatcgttatgagtgatacgctctaaccaactgagctatctcgagtattactctctgttactaattttatctatatagttaggaactaataatcgtaaacttttttaggtaggtatctaaaccatgcactgataggttctataggacgtattggtcataactatgatcatttataagcagtacgagattcgaactcatgacacactggttaagagccaggcgctctaccgctgagctaaccgcttaacaccgtacttttattaggatagggtcctataggaatgttatccatctcatgctgaccaactacgagtcgaacgtagacagactccaccaaaaaagagcagggctaccattaccccattggtcatacactgtgcaccattacctaccctcctctacccatcttttctaccctttactcccccccctttttaagggggttagtaannnnnnnnnngacagaggtaacaagatgaaatactgcttatagtgatggacagctgacaggagcacgactcgaacgtgcagtggacaatagcccgactggtttacagccagctcccttaccaattaggggtaccctgcctttgtttatacgatactgccccaacgtggagtcgaaccacggcctgctgaacttcactcagctgctctgaccaactaagccatcggggctatcaatgctctattagagattgcagatatctataacgacaggtgaaaagcaaatgctgatagttaataggcaatgaacttgctaggcacagaatgatagaaaaatattgttacaaaaatggtaagagataataatacattgtaaggaggcatagtacaatcctactctgtgcctaaacacctagaacgattcgaacgtccatctgtcgggccgaaaccgaaggctctaccattgagctataggtgcaggttcctagctatcgactatgaaaactttctcgattaggggcattgactagcactatcatcaagcataggaatatatagcctgatggggtaattactgtggtaatacatattttctatagtatttaggtaaacacgcagccttatatagtatttatacgtttgataggactcgaacctacacannnnnnnnnngctacacatctctcgatacacgctcctaaggcgcgcctggctaccaattacagcacaaacgtcatagtgttctatacactattagggcatagaatacctagaataaaatagtagatgcattgcttattaccacaactatgttactattcataatactatcaccatgcctattaattatggaaccactatgatgagttctattatctatactctaatgaatgaaaggggtctatagtatatctgttagctatgattgtagtgatggggctgtagcagctggaacatcagcagctggcacgtccaggtcagatagtgtgttttccacaataccaactacaggtacaataacaaggtaccatccgaagtagaagatacttgcaatagcaccgatagtaatgaacggttcttcagcgtgttggctaccaagccatcctagtagtactaggtcaacagctaggaaccagaacgctactcgccatagtggtcggaattgtccaccacgtacacggctagtgtccagaataggcatagctagcaggataagtagtgatccaagcatggctaccacacctagcagcttgttaggtacggcccgtaggatagcgtagtaaggaagtaggtaccactcaggcacgattgaggccggtgtactcatagggttggctgggatgtagttgtcgctgtgacctagcaggttaggtgcgtagcacacaatacctgtgatggcagcaaggaatacccacatagtcaccaggtccttgaatagatagtatggagccattggcagcttatcgctgttagatgagagtccaagagggttgcttgacccgtgtgtgtgcagcgtcatcatgtgcactagagctagtgcagatagaacgaatggtagcacaaagtgaagagagaagaatcggttaagcgtagcgttgtttacactaaatccaccccaaacaaattgtacgaagtcttgtccgatccatggaatagctgaaagcatgtttgtaatcacagtagctccccatagtgacatttgaccgtatggtagaacgtatccaaggaaagcaatagccatcataaggactaggataattacacctactgaccatggcattacccgtggtgccttgtatgaagagtagtaaagaccacgtccgatgtgggcgtatactaggatgaagaagaatgaggcaccgttagcgtgtaggtaccgtagtagccaaccgtagttaacgtcacgcatgatatgctcaacactcacaaaagccaggtctacgttaggtgtgtagtgcatcgctagaatgacaccagtcacaatttgtagacctagacatgtggctagtagtgatccaaagttccacatgtatgataggttcgcaggttgtggactgtcgacgacgtaagagtttactagtcgtagtagtgggtactgctttagcagacgcatagtacactagtaatgtccaccacatggattacaatgaagcatgatggacaggtatccccgcagggacattaagaagagagatgtcatgttaatagtgataaatctaacatgttgttaatgatagtgaggttatttagcagactatttcagtatgatacgttaccaacgctagacgtatgaggagtcgaacctcaatcaccactatgagtggtggactaccagcctaatctgatacacatcttgtacagttttagacgtaattagatatccatcacccttttaactttcgtatacctccagagactcatgtataatataagaacagtgatacgactctatagtcataagtacaggaatcccaccccttgctataaaaagatataatattaccacattcacacagacgttatcagttttatcatgtttaagaatatcctttacccggatcatacttaagagcattataatttagatgtgttgaggggctaggtccacgtagggggagtgtagtgcatcgccagaatcactcctgtaacgatttgtagacctaggcatgtagtagtgacccaaagttccacatgtatgataagttcgcaggttgtggactgtcgacgacgtaagagtttaccagtcgtagtagtgggtactgctttagcagacgcatcgttcctacttaccccccctgtaagggggggtaaggtggccatagaatggattacattaatgtccatgaccgggtaccctataggggccttaccttaaaaaataaacttccagaataggcatggtgctctgattagagggtgagaaaagaaaaatataatttcaataaatagggctattacaagctttaaagccttaacgttgccatcgctaatagacatacgaggagtcgaacctcgcccgccgctacatcaaagcggtgcactaaccagatatgctatacgtcttgtcttgttctgttatgcttagttagttaattataacgtttcagtattcaatgttattaatgcctgtaatttattacggttaactatgcttatgagcaggaatataatgaatatatttattaaaaggctggctaccttatccatattagagcaagttggctacttttatggatatttagaaaaagggtctatagtgatggtacaacatatgctaatacactatgacttgctaccttggcttactgatggtagtagccctagccctgctacgattagtagcactagagacacgtcagtaacactggccgatggtaccccaggtagtactagaggagcgatcaccataagggcaagtgataggaagcctagaacaatgtaaagggcgtagttcgttacgactgatgtgtcgtaagaagcaatagtacgtcccatgttaggcagtactgttgatagtccataaggaccaacagtttcgatcagaccacggtcaagaaccttagatgcaacgtatcccaggttcatgagaggcgtgattactagacctgttacagcagcgttccatcgccattgtccgttgatgaacccgtatagggctcgtgctagggcactgtcggttaggcttacagccagacgcataccaggaccgtataggaatacagctcccattgctccgagtagtgtcataaccagtggcagttgcttgtatagtactggtagggcaaattcagcttctaccagtgatacgtgagaagggtgttggggcagtaccgcgctgaggtagtcagtacccattcctagccataggtccttagccatgtatccaaacagtactgaaagcactgctaggaatacaagtggagcacctagtagtactggagcttcgtgcacgttgtcgtaagcttgtcgtgttccattgacagtaccaaagaacgttagggcgatcagacggatgctatagaacgctgtaagaccagcagccagtgtaccggcccagtagattactgttccgttaaggctgtatgttcccatagctagttctagtagatggtccttactgtagtaccctgtcagtcctggcagggccataagtgacagtgaaccaatcagcatagccgcgtatgtgaacggtagtagtggagctagtccacctagtcgtcgtaggtcttgttggtcagccatagcgtggataacactaccagcagccataaacagtagagccttgaaacaggcgtgtgtaacaaggtggaaaagccctacgctgaattgtgatagccctacagccatcatcatgtatcccagctgtgaacatgtagagaaagcgatcactcgctttaggtcgttcactagtagaccacatgtagcagcgtaagtagctgttgccgcaccgagccaagcagttactacaagggcggtaggaccgtactctagtagtggtgatgtacgaagcataaggtataccccagcagtcaccagcgtagcagcgtgaattagagcagacacaggagttggaccctccatggccaataattgttagtagcaatgacaatccctttcaggattgagcaggccatgtcaaccatcaggaagtacttatatgcaaaaaaatatcttgaatcatgtttaagcataacaacctatgggtactagtatggtctttgggcatgaatcacaactagtgattctatgctgccagtattgaatagttacaaatactggcaaatcatagtacagcagacgctttaaaaccaagacagaaaaaggggactatcgtacaagatgccgagcccatacgggtagttgagcaccaataagctcgtctagtacatgaaggtgttcagtaggtacccaaacgtagtagagagtggtacgtcgaccatgtggggctgggcgttgggaagacagggctggtacacctagggatcgtagtcgtgcagcgaataggtcgtattcgtaatcttcagcaccactccaaaggggtagtaccagtccgcctgctggttgtgagaagtactcacgccaagtgtctcgataagcgctcagaataatatgaacaacaagagaatgaagattgtagcaacccagtgcttgggctgttagacgtcgtacaccagggcctgcaggcgtcaggtagaagagtgaatacagaggccatagtgctggtagtgtacttgtgagcatcagagtgaagctgaccggtgggaggtgaggacgacggttatctgtcacctcagtagtaacaggcttattgaccataaacatggtcattagtgattgaacatattcccactcagtttggctccacatagaagaaatacgcttgattgatagccgtgctgtgggtagaccactagtcaggtacttgctgaacatttggaatggccccgccacgagcatagctacgaaagctgcaagttgatcagggctcatagaggtgggcatatccttcttagcactagcacccttgttaccatacgaaccaggattagtaccaggacctgtcttttcacctgaacttgatggagtagtacccgtacccacctttgtgggatctgtcccggtaataagatttagttcatcagctagccacgtatgtagtggtagttgagcactcttactcatagccccacctaggaagagtagaccaataacagttagtactgtttcgtttagaagaggtgctgtgctcagaatagttgcgtagtccaggctaccaaagacagcgagagccaggaagaaaccaatgctgagtgaggtgtcaccaacacggttaaccatcatggccttgttagcggccttagacgcttgtacccgtgtgagccagaacccaataaggaggaacgatgccaccccaataagttcccaaccaatgaacatcagtgggtatgaatccgctgttaccagcagagccatgctcccagtgaacagcgaaagatagctgaagaatcgtggtgtgtgtgggtcttcagccatgtatgacatgctatagacgtgtacagcgctagatacgcataggacaggtagaagcatcgcaactgtaaggtcatcgaagctaaatgcccattgagcttgtagcacaccaacggtaagccagtcacctacaacgatagtaacaggtgaacggcaaagagctacttcgtagaaggctactagtgatagacatgccgatagtactaggcatacagtggcaactagttgtgaacctgtgctaccaagtgctcgccctcgtagaccagcggctgctgaacctaggaagggtagaagaataagagctaggtacatagactatagtcgtggagatgataggctcagtgtcccccgtaggcggtaataggcaactagtactccaagaccaatagcgctttcagctcccgccgtagcgatgataatgatactgaatgtcattccagcagcatcgctatagctgttagctgttgtgagaacgagtagtgtcaccgcgagcagcatcagttcaatgctcacaagtagcatgaggaggctacgccggttaaggctaaaccccagcacaccaacaaggaacagtacgagtgatagagtcatgggacagtcattcgtgataggcggaaggccatgtttaaaacatcgtacagtaggagtacttggcatccatgatagtgaccatatcactcacttcccccccgaaagggggtagtggtaagtagcagactgttgccaaacagactacctatatcagtagtatagtatcagtacgtggaccagcaacattgagttacgggcgacccactaactctctccaccctttacccccctatgagggggggttgaagggataggtaaaaggatttagtaagaagggatgtttaaaagatgggtaaatatattattataaggtagggtttagattatgtgggagattattataagaatcccgcggcagatgggactcgaacccacactcacaacgttgacaacgttggacactgccattatgctactaccgctaggcactgtcctattactctaattactatctttgtgataatacagttgtattagtatagtaacaataaaaggtatcatctgacnnnnnnnnnnccttttggtgaaaggagaaaagaaaaatctataccgctggtacgttgtagagcagtgcagaagcttcaacttctagaccagacagtagtacgccagggatcacaccaaggaatagagtcatgacgatcagaggtagaactgtcatagcttctgttcgtgtcaggtcacctgcgattgttaggtatggacttagagttcctgcgctgatacgggcccatagccatgtagagtagcaagcgctaagcacgatacctgtagctccaagagcacaagctaggaatgattgttccatcaccctgctagagccatgaattcacccacccagttgactgagagaggaacacccatgttagcacagatagcaaagaagaacactactgtgaatactggcataacgctagccatacctcggtagtagctgatagaccgtgtgtggtatcggttgtatagaacaccacccaccatcacgaatagcgctggtgagattagaccgtgcgctagtgatagtagtagagcaccactgataccaattacgctgttactgaagagccctaggacgaccacagccatgtgccctactgatgagtatgcaataagctgcttagtgtctactgaccgtagtgtggctagactagagtagatcagacttacaacagcaagtgtttgtactagaggtgcgtaatagtcgcaagcatcaggtagtactggtagtactagtcggtatactccatacgtggcaagcttcagcactgtaccagctagaagcatagatccaggcagtggagcgtcagcgtgagcacgaggtagccagatatggaaaggcacgattggagtctttaccataagggctagaccaaagcacacaaacaggtatcgttgtacagcagggtctagtgatacgtgttgtagcactgtcatgtctgttgtacctgtgtggctatatagacctagaatacctagtagcatgaatagagatcctgctagtgtatataggaatagaagtagggctgaccgtactcgtgttggtgagccaccatagagaccaaccatcacgtaaagaggcatcagtacagcttcgaatgctacgtagaagacgatgtagtcagtagtagtgaatgcagcgttaataagagctgtagcaaggaacacaagagccagttgggccttttgtgaagcgtgaggagtgtcagctagagctaggaccataggttctactaggaagcatgaaagtgttactagtagcatgctagtgtagtccacaccaaagagaagaggagcaccaataccaataaattcgtggtattccatagctccccaggggtcagtagccagtagcatcagacagaccatgtaacgaagtgtttgtgacgacgccatggccacagtcatagtaacgccacctggttgttctggattagctccaccttcagtaggttgccatgccaggtagatggcggtgataataggatatgcaattagatatgtaatcatagattttattaggggggtagtagccttagtagaatggccatttgtccctctaccctgttagttaataaataaagctctgcttgaatattatagaagtacttattattatgatgatagaaccgtttatggcgataaacctatattatacgtatagcagtagagttctactaaacctttaacgatagagcacaatttatttatacctataaccatagggcccatgtttatgaagataggttcttgtattatgtttatggtgatagaacctatatatatatatatatacggttactcaccattgtttataagaggtagccggatcgaacggctgctagcgctgtgtaaaagcgctgacctaaccactagtcgaacctccttgtactgttaacattatctgagcctattaatgattagaaaatatactatttagtaccattagcatagctaggagaatacagggatacatgggatcgaaccacgcctaagagctttggagactcttgtactaccactatacgatatccctatcaggcttaggaccggacataatatctatcatcttaatagataggggggctatcatgcctattcacaacagatcatgatagtatgataagctatgggtcaagccgcgggctcgaaccgcgcacctctccgaccacaacggaacgctctgccaaatgagctagaatgacctgtgttatggtcataccaatacaccagaggaataataatgcttagaggcaataatatattattcaatactaatagtttcctctgtttgcctccgcgtgggatcgaaccacgattttaggtttacaagacccacgttgcaccattcaactacagaggctcatagtgatcgataaagaccccatatctatgaccattgtaacgctactgggacttgaacccagtctacctgtgtgaaggacaggtgtgctacccctacaccatagcgtctactactgttatatatgacatactatgttcatgagcacgcgcacataaaaatatccctaataatataacacttgagctatggtaaatagtgggggtctattattatgctacccattactaccctttttaggttagttaaggaaaagtaaagcggannnnnnnnnntagctaggagaatacagggatacatgggatcgaaccacgcctaagagctttggagactcttgtactaccactatacgatatccctatcaggcttaggaccggacataatatctatcatcttaatagataggggggctatcatgcctattcacaacagatcatgatagtatgataagctatgggtcaagccgcgggctcgaaccgcgcacctctccgaccacaacggaacgctctgccaaatgagctagaatgacctgtgttatggtcataccaatacaccagaggaataataatgcttagaggcaataatatattattcaatactaatagtttcctctgtttgcctccgcgtgggatcgaaccacgattttaggtttacaagacccacgttgcaccattcaactacagaggctcatagtgatcgataaagaccccatatctatgaccattgtaacgctactgggacttgaacccagtctacctgtgtgaaggacaggtgtgctacccctacaccatagcgtctactactgttatatatgacatactatgttcatgagcacgcgcacataaaaatatccctaataatataacacttgagctatggtaaatagtgggggtctattattatgctacccattactaccctttttaggttagttaaggaaaagtaaagcggannnnnnnnnngtaaagcggagccaggactcgaaccatggacctacagctcatgaggctgcaatgctgccaaattacactaccccgcgattgattatgaccatattatatataaagacaggtttatctccgtactcggacgcgataggagtcgaacctacaactggtcatcccagatacagctagcaaccgtatacacctcaccaatagttgtgcacgtcccgtcctgttacacataggtgagtaagcctataataatgggtcatactattaacgataggttaaattaaatcctataacaacgggacatgttattagcgatgttgttgttttcttgttattcatgattaggtttgccaacaatagtgtgtggtaaaaagggtgaggggtcattaggtagttggtatgggtattggacttatgggtgtgactacgctctggacgcatgcccattacacgtgtcacccctttactatctttgatagttaatcttgcgcagtagtctactgcgtgtccatagcgtttgaacatcgttattagcaatgttcaaacgcgttgatgcatgtccatagctggattcgggctttatatgctggcagcccttatccattgggtgcttagctgccctgcggtggctcattaataggcacaacaggtacaccagaggtaccctgacactggtcctttcgtactaggtgccatcctcgaacatgctaataaagcacagcgggtataagctatactgcttcacagcgtattgacaatagtgttattcataataaaacagtggctttcaccactggtcagactctatcataggcagcaagcttgctaccgtgcaggatagttataggctctgagccttatagtcgttggacgtgatactttagaataattcacgctcctagttgtaaggtatatacttccaggatggtttctgcttttggtgatcaatcaccgttgggctcaatttacattattctcaacaaaactatgacttaaaacaaaggggtctagtttaggtcaactcgaggacctaggcggtacagcatagattcatgcatatgaggtgtaagcatggcttgtagtgccttgtgatgggcctttgtaatcgcaagcttgtattgcttttgtccatcctttccataacggtcagcacggacagtagtggggatacccatggcagtaatggccgctgctaggcgtacgcaatctgcatgagtatagctatttacgtagatgaatacggtactgtttgcgatgtcatatgagccgtcactcatgatgagatgggccagtacaacaggcgtcacaagatccttaatcatagccggaactaccttgatccactcaccagcagcccccatcacataaaacatattgtgatattggttgaaaacgccgagagaaagagtcttcagccggaattgtccagagggtagtgtagtaacccccgtttggcagaactggctaaatagtccatatacccaataggcataatcacgataaggggccccaaaagaccattccaggcgtgtattagatgtagcacttgaacgcgatgcatggctatcgcccagaagttgtccaataaccacttggtgtaggtaatatgggagcattacaatggatcgtagaccccgaataatttcgcttgtaaagcgttgaagaagttgtagtgttgatgtcatagttgttttttgttgattgaattaaatatatgtcgcttaagcgaacccaactcacgtagctccttagactgcgaacagcagtacccttccaagcttgtgcacctggaggatgagctgagtcgacatcgaggtcgcaaacagcacgggcgatatgaactctcgcgtgctataacgctgttatccctagcgtagcgttaatcgcctagcccagagtgtaccctgacacgtctggggttcacttggccatgcttgcgcactttaagcactgctcagtgctatcaatcaggcacccattcatgtccatgaacgatagtagcccctacatgggcaataccttaaaaaaggctattcgggtttccatcccgggcaagggtgccatacgtggaggcctccggtactgtataggaggcgaccgtcccagtcaaactagctgatgacctatcctagcccattgcccgtaagagcggggcttcactaccacgaatcctcatggcggtgtaatagtcgaacgacatacttccccagattcatgtggcgaccggccatcactagtaaagctgcatagggtcttcttgccctgctgtgctaaaaccgcatcttcacggcttattcagtttcgctgaggggcccccggagacagcgggtgcgtcgtgaccctcttcatgcgggaccataattagtggccaaggaatttcgctaccttagcacccttatggttaaggcggtcgtttacctgagttaggtacacgttgaagcccttaagctctacgcagtacattcactctaggcaccggacaagggacggcccctatacgagccacagctggtgctgggcatagcagggacctgtgttcttggtgcacagtcgcacaccccgttccactgcgtccagccaggatgtttaatttctaagttaaaactaagcttaaaaaataaactatccccataagctggacacccttatagtaggacgtacgggtttctgttgccgagttcctttgggagccctatctctcactctggtgtactccaccacgggacctgtgtcggtcataggcacggtaaacgcacgcagcttcaaggcatatttattatatagtatgctcagcttgtgttaactcatcgctttatacttagagaccggtttaactcaggatgactgccattgtctatctgagacccaccccgttcaagtgcaaggtgtctagaccttgtatcgctactcgtgtcgccatgctctatccacgttgtccagacaatgctacactgcccttctttcaacatgggtgctcatctaccctataatcttactacatcattattattatagcccgcacccgcccatgttacaaacccctttgggggtaagagcaggtgaggcatgtgaactttagacataaatacccctatctttcatgaaaaagataggtaaataatattatatgtttacagttttagtaagtatttataggcgttgtgtgggacgatagctgagacccgtttatcttcggcgcactggtactcggccactgggttgttacaccatcgttaacaggtggctacctccaggcacactgcatgactgtcattgtagcagcacctccttacgtgtcatttagctatatctgtttgggtcctcgctcaacgctcatgactgtcttcatctaggctgctgaccttatcgcctacagcctgtgtacaggacgtagtagtcccccttccatggtaagctacccctgatagagcacggatgccccccagaactgcactcatttcctataaaacctcctagatggtgatcttatattaaataaatacatggcggcctgaacagtacagggtcctaccactcgatgcctgtctgacgccttaacgtctttcgatgagagccggctagctcgctgttagataggcctatcacccctagcctgagctcgtcagagtacaatacaacggacacctgttcggcctctgtcatggccatcacacgtcaatcaagtacttaaaatacttggacgtaagaggacacaacaggcctggcccaagctagatcacagcggttcaggtctgggcaggaggactaattcccctattgcaccataataagcaccaatatagcctattgactataactgacacatatcacatcattacctttatcaatcatttctattgtaatgatccatacctgatttcaccaatttatagtgtagctatatctgacggtttatctgatataacaacaaactaaaactgatggtcaatggcttatattggtaggagatgtatggatagcaatgggcgattttgtctgtcgtttaggctattggcctcgccctccttcccaactcggcgacccattatgcaaaaggtacgctgtcgggcgttactatggcgatcctagaaacccgaccatagcattgcagcccttcagctgcttatggaccatagctgcttcaggctctatttcactgcggtacaaccgctctctttcatcgttcactcgcgttactattttctatcactcggaccacctattagccgttgggagagatcccaatcactctacaaaatgcagaatgctctttaacacatcagcagtcacacgggacttttaccctctatggttatctggcgttccatccagttcaaactgtcgatgacatgttacagctacacctacttttcgctcgccgctactaaggaggtacggttggttgcctgtaggccctactatgatgattcactttgggccatggctatcattgatttaccgtgaggttgtcattaattgacctttggactagggcnnnnnnnnnncaacaaaagatagtgtgtttcctactactcattactaaccccttctaattagaattaccctaaccccaccaaaggggggtaactggtgggttaaaaggtaggtaaaagtaatagttactccactagtcataatggttataggtgtaggtactcactcatgaaattatgaccatgagtgttcataaataactatgaccatgagtataactagtagtattggtggttaaaaagtttaatggtaatagaatatggcttatgcttatgatgggtggggtctataatacatgaatgtactatggacacccgggtttggtggtcataat

SEQ ID NO: 3 - DNA, 5948 bp, Papiliotrema terrestris
  misc_feature (432)..(441): n is a, c, g, or t
  misc_feature (1082)..(1091): n is a, c, g, or t
  misc_feature (3668)..(3677): n is a, c, g, or t
  misc_feature (4184)..(4193): n is a, c, g, or t
  misc_feature (4824)..(4833): n is a, c, g, or t
  misc_feature (5474)..(5483): n is a, c, g, or t
  misc_feature (5892)..(5901): n is a, c, g, or t
acctggttgatcctgccagtagtcatatgcttgtctcaaagattaagccatgcatgtctaagtataaacaaattcatactgtgaaactgcgaatggctcattaaatcagttatagtttatttgatggtatcttgctacatggataactgtggtaattctagagctaatacatgctgaaaagccccgacttctggaaggggtgtatttattagataaaaaaccaatgggtgcaagccctctatggtgattcataataacttctcgaatcgcatggccttgcgccggcgatgcttcattcaaatatctgccctatcaactttcgatggtaggataggggcctaccatggtatcaacgggtaacgggggaattagggttcgattccggagagggagcctgagaaacggctaccacatccaaggaaggcagcaggnnnnnnnnnnatcccgacacggggaggtagtgacaataaataacaatatagggccctattgggtcttataattggaatgagtacaatttaaatcccttaacgaggaacaactggagggcaagtctggtgccagcagccgcggtaattccagctccagtagcgtatattaaagttgttgcagttaaaaagctcgtagtcgaacttcgggcctggctgggcggtccgccttacggtgtgtactgtccggccgggccttacctcatggtgagcccgtatgccctttactgggtgtgcggtggaaccatgaattttaccttgagaaaattagagtgttcaaagcaggcataagcccgaatacattagcatggaataatagaataggacgtgcggttctattttgttggtttctaggatcgccgtaatgattaatagggacggtcgggggcattagtattcagttgctagaggtgaaattcttagatttactgaagactaacttctgcgaaagcatttgccaaggacgttttcattgatcaagaacgaaggttaggggatcaaaaacgattagataccgttgtagtcttaacagtaaactatgccgactagggatcgggccacgttaatttctgactggctcggcaccttacgagnnnnnnnnnnttctggggggagtatggtcgcaaggctgaaacttaaaggaattgacggaagggcaccaccaggtgtggagcctgcggcttaatttgactcaacacggggaaactcaccaggtccagacatagtaaggattgacagattgatagctctttcttgattctatgggtggtggtgcatggccgttcttagttggtggagtgatttgtctggttaattccgataacgaacgagaccttaacctgctaaatagtcaggccggcttcggctggtcgtcgacttcttagagggactgtcggcgtttagccgacggaagtttgaggcaataacaggtctgtgatgcccttagatgttctgggccgcacgcgctacactgactgagccagcgagtttatcaccttagccgagaggcttgggtaatcttgtgaaactcagtcgtgctggggatagagcattgcaattattgctcttcaacgaggaatacctagtaagcgtaagtcatcaacttgcgttgattacgtccctgccctttgtacacaccgcccgtcgctactaccgattgaatggcttggtgagatttccggattggcgttggggagccggcaacggcacccttggctgagaagctactcaaacctggtcatttagaggaagtaaaagtcgtaacaaggtttccgtaggtgaacctgcggaaggatcattattgattggtcgaaagaccttatcagattctaccacctctgtgaaccgttgacctccgggttaataatcaaacatcagtgtaacgaacgtaagagtatcttaacgaaacaaaactttcaacaacggatctcttggctctcgcatcgatgaagaacgcagcgaaatgcgataagtaatgtgaattgcagaattcagtgaatcatcgaatctttgaacgcaccttgcgccttttggtattccgaaaggcatgcctgtttcagtgtcatgaaatctcaatctaatatgttttctgaacatgttagacttggacttgggcgtctgccagtgatggctcgcctcaaatgacttagtggaacatcccacatcagtgttagacgtaataagtttcgtctctccttgtggtgatgactgctcagaacctgccatcgcgcacttttgactttgacctgaaatcaggtagggctacccgctgaacttaagcatatcaataagcggaggaaaagaaactaacaaggattccctagtaacggcgagtgaaccgggaagagctcaaatttgaaatctggcgtgctcagtgcgtccgagttgtaatctatagaggcgttttccgtgccggactgtgtctaagtcccttggaacagggtatcaaagagggtgataatcccgcacttgacacaatgaccggtgctctgtgatacgtcttctacgagtcgagttgtttgggaatgcagctcaaaatgggtggtgagttccatctaaagctaaatattggcgagagaccgatagcgaacaagtaccgtgagggaaagatgaaaagcactttggaaagagagttaaacagtacgtgaaattgttgaaagggaaacgattgaagtcagtcgtgactgagaggctcagccggttctgccggtgtattccctcagtcgggtcaacatcagttttgttcggtggataagggcagctggaaggtggcacctccgggtgtgttatagccagctgtcgcatacatcgaatgagactgaggaatgcagctcgcctttatggcggggttcgcccacgtccgagcttaggatgttgacataatggctttaaacgacccgtcttgaaacacggaccaaggagtctaacatatctgcgagtgtttgggtgtcaaacccaagcgcgtaatgaaagtaaacgtaggagggatccgcaaggagcaccttcgaccgatctggatcttctgtgatggatttgagtaagagcatatatgctgggacccgaaagatggtgaactatgcctgaatagggcgaagccagggaaactctggtggaggctcgtagcgattctgacgtgcaaatcgatcgtcaaatttgggtataggggcgaaagactaatcgaaccatctagtagctggttcctgccgaagtttcctcaggatagcagaaactcgcatcagttttatgaggtaaagcgaatgattagaggaattggggacgaaacgtccttaacctattctcaaactttaaatgtgtaagaaggacttgtcacttaattggacgagtccatgcgaatgagagtttctagtgggccatttttggtaagcagaactggcgatgcgggatgaaccgatcgtgaggttaaggtgccggaatacacgctcatcagacaccacaaaggtgttagttcatctagacagcaggacggtggccatggaagtcggaatccgctaaggagtgtgtaacaactcacctgccgaatgaactagccctgaaaatggatggcgctcaagcgtgttacccatacctcaccgtcggcgttgaagtgacgcgccgacgagtaggcaggcgtggaggtttgtgaagaagccttggcagtgatgctgggtgaaacagcctctnnnnnnnnnnagatagggaagctccgtttcaaagtgcacgcttatccgtgccgcctatcgaaagggaatccggttaagattccggaaccaggatgtggatcattgacggcaacgtaaatgaagttggagacgctggcaagggccccgggaagagttctcttttctccttgaccgcttacgacctcgaaatcggattatccggagatgaggttatatggcgggcagagcacgacacctctgtcgtgtccggtgcgtccttgacagtccttgaaaatccgacggaacgtataagtctcacgcctggtcgtactcataaccgcagcaggtctccaaggtgaacagcctctagttgatagaacaatgtagataagggaagtcggcaaaatagatccgtaacttcgggaaaaggattggctctaagggttgggtacgtcgggccattggcggaacagagctggaccaggtcggacttgctggggcaacccggctggactggctcggaccggcgatgggatgnnnnnnnnnnaactatgactctcttaaggtagccaaatgcctcgtcatctaattagtgacgcgcatgaatggattaacgagattcccactgtccctatctactatctagcgaaaccacagccaagggaacgggcttggcagaatcagcggggaaagaagaccctgttgagcttgactctagtttgacattgtgaaaagacatggagggtgtagaataagtgggagcttcggcgccggtgaaataccactacctccatcgtttttttacttattcaatgaggcggagctgggattaacgtcccacctttttgtcttaaggtcctttacgggctgatccgggttgaagacattgtcaggtggggagtttggctggggcggcacatctgttaaaagataacgcaggtgtcctaagggggactcatggagaacagaaatctccagtagaacaaaagggtaaaagtccccttgattttgattttcagtgtgaatacaaaccatgaaagtgtggcctatcgatcctttagtccctcggaactcgaggctagaggtgccagaaaagttaccacagggataactggcttgtggcagccaagcgttcatagcgacgttgctttttgatccttcgatgtcggctcttcctnnnnnnnnnnggagtaactatgactctcttaaggtagccaaatgcctcgtcatctaattagtgacgcgcatgaatggattaacgagattcccactgtccctatctactatctagcgaaaccacagccaagggaacgggcttggcagaatcagcggggaaagaagaccctgttgagcttgactctagtttgacattgtgaaaagacatggagggtgtagaataagtgggagcttcggcgccggtgaaataccactacctccatcgtttttttttacttattcaatgaggcggagctgggattaacgtcccacctttttgtcttaaggtcctttacgggctgatccgggttgaagacattgtcaggtggggagtttggctggggcggcacatctgttaaaagataacgcaggtgtcctaagggggactcatggagaacagaaatctccagtagaacaaaagggtaaaagtccccttgattttgattttcagtgtgaatacaaaccatgaaagtgtggcctatcgatcctttagtccctcggaactcgaggctagaggtgccagaaaagttaccacagggataactggcttgtggcagccaagcgttcatagcgacgttgctttttgatccttcgatgtcggctcttcctgtcnnnnnnnnnnagaattcggtaagcgttggattgttcacccactaatagggaacgtgagctgggtttagaccgtcgtgagacaggttagttttaccctactgatggagggttatcgtaacagtaattgagggtagtacgagaggaactgctcattcagataattggtatttgcgcctgtccgatcgggcaatggcgcgaagctatcatctgctagattatggctgaacgcctctaagtcagaatctgtactggaaacgatgatgttggtcccgcatgtgttagttgtgtcaaaataggcttcggctgtgaaccatatctgggctgggttgtttggacggaaaggtccttgcagcttgctcttgattgaaatggaatatacgcggggggtgaatcctttgcagacgacttgaattggaacnnnnnnnnnntagtagagtagccttgttgctacgatctactgaggctaagcccttgt

SEQ ID NO: 4 - DNA, 1587 bp, Papiliotrema terrestris
atggctagccgatggctcttctctacaaacgccaaggatattggtaccctttatatcctttttgctgtctttaccggtcttctgggtactgcgttcagtgtacttatccggatggagctatcagctcctggtaaccagttcctttctggtaaccaccacctctacaacgtaattgctacaactcacggtatcatgatgatttacttcatggtggtacctgctatggctgggttcgctaactacatggctcccgttcttatcggtgcgccggacatggcgttcccacggctgaacaacatcagcttctggctactacctcctgcaatcgttcttatcctttcaagcgtcttcgtggaacaaggtatgggtagcggttggactatgtacatgcctctaacaggtgtacaatcacacagtggtggatcagttgatctagctgtattcagcctgcacctctcagggatttcatctctacttggtgccattaacattatcactacaatccttaacatgcgagctccagggctacgcctacacaaggcacctctattcgtatgggcgatgctatcacagtcagttattattattctatgtattccagttctagccggagctcttactatgatccttactgaccgtaacttcaacacatcattcttcgaccctgctggaggaggggaccctgtcctataccaacacctattctggttcttcggacaccctgaagtatacctaatgattatccctggatttggtatggttagccacgttgtttcaacattcagtggaaagcctgttttcggttaccttggtatggtgtacgccattgcctctatcggtgtgcttggattcattgtatggagccaccacatgtacgccgtagggatggacgtagacacacgagcttacttcacagctgcatctatgattattgcggtacctacaggtatcaaggtcttcagttggctagcgactgcttacggtggttctatccgactgctagcacctatgctattcgctctaggattcgtcggtctgttcactattggtggactgacaggtgtagttctagcgaacgcgagcgttgacgtagcgatgcatgacacatactacgttgtggctcacttccactacgtgctttcaatgggtgccgtattcagtatctttgccgggttctactactgggctccaaagatgttcggtaagatgtacgaagaacacctagctcaagcgcacttctggacattcttcatcggtgttaacatgacatttatgccacaacacttcctaggactacaaggaatgccacgacgattcgctgactaccctgacgcttacgcgggatggaacctaatctcatcatggggttcattcgttagcgtctctgctactcttctgttcctgtacactgtttacgacatgttcactaacgacaagtacgttggtgacaacccatggggagaaccgctatacttcacggacaacctaacgtacgaacgaagcacacggttcgcctacacaatcgagtgggtactaccatcacctactccacaccacgcattcgacatgctacctatccaatcatag